(19) United States
(12) Patent Application Publication
(10) Pub. No.: US 2025/0215493 A1
(43) Pub. Date: Jul. 3, 2025

(54) NUCLEOTIDES WITH ENZYMATICALLY CLEAVABLE 3'-O-GLYCOSIDE BLOCKING GROUPS FOR SEQUENCING

(71) Applicant: Illumina, Inc., San Diego, CA (US)

(72) Inventors: Stephane Emond, Cambridge (GB); Antoine Francais, Cambridge (GB); Adam Culley, Cambridge (GB); Cassie Zerbe, Cambridge (GB); Natasha Crake, Cambridge (GB); Elena Cressina, Cambridge (GB); Alexandre Hofer, Cambridge (GB)

(21) Appl. No.: 18/990,918

(22) Filed: Dec. 20, 2024

Related U.S. Application Data
(60) Provisional application No. 63/615,477, filed on Dec. 28, 2023.

(51) Int. Cl. C12Q 1/6874; C07H 19/073; C12Q 1/6806; C12Q 1/6823

(52) U.S. Cl. CPC C12Q 1/6874; C07H 19/073; C12Q 1/6823; C12Q 1/6806; C12Q 2525/186

(57) ABSTRACT

Embodiments of the present disclosure relate to nucleotide and nucleoside molecules with 3′-O-glycoside blocking groups. Also provided herein are methods to prepare such nucleotide and nucleoside molecules, and methods and kits for sequencing applications.

### CROSS-REFERENCE TO RELATED APPLICATION

The present application claims the benefit of priority to U.S. provisional application No. 63/615,477, filed Dec. 28, 2023, the content of which is incorporated by reference in its entirety.

### REFERENCE TO SEQUENCE LISTING

The present application is being filed along with a Sequence Listing in electronic format. The Sequence Listing is provided as a file entitled “ILLINC824A_Sequence_Listing.xml” created on Dec. 17, 2024, which is 2.88 KB in size. The information in the electronic format of the Sequence Listing is incorporated herein by reference in its entirety.

### BACKGROUND

### Field

The present disclosure generally relates to nucleotides, nucleosides, or oligonucleotides comprising 3′-O-glycoside blocking groups and their uses in polynucleotide sequencing methods. Methods of preparing the 3′-O-glycoside blocked nucleotides, nucleosides, or oligonucleotides are also disclosed.

### Description of the Related Art

Advances in the study of molecules have been led, in part, by improvement in technologies used to characterize the molecules or their biological reactions. In particular, the study of the nucleic acids DNA and RNA has benefited from developing technologies used for sequence analysis and the study of hybridization events.

An example of the technologies that have improved the study of nucleic acids is the development of fabricated arrays of immobilized nucleic acids. These arrays consist typically of a high-density matrix of polynucleotides immobilized onto a solid support material. See, e.g., Fodor et al., Trends Biotech. 12: 19-26, 1994, which describes ways of assembling the nucleic acids using a chemically sensitized glass surface protected by a mask, but exposed at defined areas to allow attachment of suitably modified nucleotide phosphoramidites. Fabricated arrays can also be manufactured by the technique of “spotting” known polynucleotides onto a solid support at predetermined positions (e.g., Stimpson et al., Proc. Natl. Acad. Sci. 92: 6379-6383, 1995).

One way of determining the nucleotide sequence of a nucleic acid bound to an array is called “sequencing by synthesis” or “SBS”. This technique for determining the sequence of DNA ideally requires the controlled (i.e., one at a time) incorporation of the correct complementary nucleotide opposite the nucleic acid being sequenced. This allows for accurate sequencing by adding nucleotides in multiple cycles as each nucleotide residue is sequenced one at a time, thus preventing an uncontrolled series of incorporations from occurring. The incorporated nucleotide is read using an appropriate label attached thereto before removal of the label moiety and the subsequent next round of sequencing.

In order to ensure that only a single incorporation occurs, a structural modification (“blocking group” or “protecting group”) is included in each labeled nucleotide that is added to the growing chain to ensure that only one nucleotide is incorporated. After the nucleotide with the protecting group has been added, the protecting group is then removed, under reaction conditions which do not interfere with the integrity of the DNA being sequenced. The sequencing cycle can then continue with the incorporation of the next protected, labeled nucleotide.

To be useful in DNA sequencing, nucleotides, which are usually nucleotide triphosphates, generally require a 3′-hydroxy protecting group so as to prevent the polymerase used to incorporate it into a polynucleotide chain from continuing to replicate once the base on the nucleotide is added. There are many limitations on the types of groups that can be added onto a nucleotide and still be suitable. The protecting group should prevent additional nucleotide molecules from being added to the polynucleotide chain whilst simultaneously being easily removable from the sugar moiety without causing damage to the polynucleotide chain. Furthermore, the modified nucleotide needs to be compatible with the polymerase or another appropriate enzyme used to incorporate it into the polynucleotide chain. The ideal protecting group must therefore exhibit long-term stability, be efficiently incorporated by the polymerase enzyme, cause blocking of secondary or further nucleotide incorporation, and have the ability to be removed under mild conditions that do not cause damage to the polynucleotide structure, preferably under aqueous conditions.

Reversible protecting groups have been described previously. For example, Metzker et al., (Nucleic Acids Research, 22 (20): 4259-4267, 1994) discloses the synthesis and use of eight 3′-modified 2-deoxyribonucleoside 5′-triphosphates (3′-modified dNTPs) and testing in two DNA template assays for incorporation activity. WO 2002/029003 describes a sequencing method which may include the use of an allyl protecting group to cap the 3′-OH group on a growing strand of DNA in a polymerase reaction.

In addition, the development of a number of reversible protecting groups and methods of deprotecting them under DNA compatible conditions was previously reported in International Application Publication Nos. WO 2004/018497, WO 2014/139596, and U.S. Pub. No. 2020/0216891 A1, each of which is hereby incorporated by reference in its entirety.

### SUMMARY

The present disclosure provides next-generation sequencing kits, methods, systems, and compositions.

One aspect of the present disclosure relates to a nucleotide comprising a ribose or 2′ deoxyribose having an enzymatically removable 3′ blocking group in the form of a 3′-O-glycoside group, a nucleobase and a triphosphate moiety, wherein the 3′ blocking group forms an —O-glycosidic bond with the 3′ carbon atom of the nucleotide.

Another aspect of the present disclosure relates to an oligonucleotide or polynucleotide comprising a nucleotide in accordance with the present disclosure incorporated therein.

Another aspect of the present disclosure relates to a kit comprising one or more nucleotide in accordance with the present disclosure.

Another aspect of the of the present disclosure relates to a method of preparing a growing polynucleotide complementary to a target single-stranded polynucleotide in a sequencing reaction, comprising incorporating a nucleotide having an enzymatically removable 3′ blocking group as described in the present disclosure into a growing complementary polynucleotide, wherein the incorporation of the nucleotide prevents the introduction of any subsequent nucleotide into the growing complementary polynucleotide.

Another aspect of the present disclosure relates to a method of determining the sequences of a plurality of target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an aqueous solution comprising DNA polymerase and one or more of four different types of nucleotides A, G, C, and T or U under conditions suitable for DNA polymerase-mediated primer extension, wherein each type of nucleotides has a 3′ blocking group and at least one type of nucleotide is a nucleotide having an enzymatically removable 3′ blocking group as described in the present disclosure, and wherein each of the one or more of four different type of nucleotides comprises a 2′ deoxyribose;
(c) incorporating one type of nucleotides into the sequencing primers to produce extended copy polynucleotides; and
(d) imaging the solid support and performing one or more fluorescent measurements of the extended copy polynucleotides; and
(e) removing the 3′ blocking group from the nucleotides incorporated into the extended copy polynucleotides.

### BRIEF DESCRIPTION OF THE DRAWINGS

FIG. 1 is a line chart illustrating the remaining blocked nucleoside as a function of time for an exemplary 3′-O-glycoside blocking group 3′-O-α-L-arabinofuranoside (also known as 3′-ABF) according to an embodiment of the present disclosure, as compared to two nucleosides each having a chemically-cleavable blocking group (3′-O—CH₂N₃ also known as 3′-AZM) and 3′-OCH₂OCH₂CH═CH₂ (also known as 3′-AOM) respectively.

FIGS. 2A-2E illustrate the percentage deprotection of 3′-ABF thymidine (dT) as a function of time during exposure to various arabinofuranosidases (ABFases).

FIG. 3A is a reaction workflow mimicking a sequencing by synthesis cycle as described in Example 5, in which 3′-ABF-blocked deoxythymidine triphosphate (dTTP) is incorporated into a growing polynucleotide, followed by enzymatic deblocking and the subsequent incorporation of a 3′-AOM blocked deoxycytidine triphosphate (dCTP).

FIG. 3B is a gel electrophoresis image of cleaved 5′-fluorescently labeled primers which were subjected to the experimental conditions illustrated in FIG. 3A.

FIG. 4A is a sequencing assay workflow for monitoring incorporation and subsequent cleavage of an unlabeled 3′ blocked dTTP using loss and recovery of % T call signal as readout as described in Example 6.

FIG. 4B plots % T call signal using unlabeled 3′-ABF blocked dTTP over the course of the SBS workflow described in FIG. 4A.

FIG. 5A illustrates the experimental workflow used to perform surface kinetics of 3′-ABF enzymatic cleavage from DNA as described in Example 7.

FIG. 5B shows the kinetic plot of 3′-ABF cleavage by ABFase over reaction contact time.

FIG. 6A is a gel electrophoresis image of ³³P-labeled DNA primers incorporated with various 3′-O-glycoside thymidine triphosphates according to certain embodiments of the present disclosure.

FIG. 6B is a chart showing percentage of incorporation as a function of time of various 3′-O-glycoside thymidine triphosphates.

FIG. 7 is a line chart illustrating the cleavage kinetics of 3′-beta-glucopyranoside group by various β-glucosidases at various conditions.

FIG. 8 is a line chart illustrating the cleavage kinetics of 3′-beta xylopyranoside group by various β-xylosidases at various conditions

### DETAILED DESCRIPTION

Embodiments of the present disclosure relate to nucleosides and nucleotides with 3′-O-glycoside blocking groups for sequencing applications, for example, sequencing-by-synthesis (SBS). These blocking groups allow for enzymatic cleavage of the 3′-O-glycoside blocking groups (also referred to as 3′ reversible terminators). In particular, enzymatic cleavage is likely to be less damaging to DNA and may generate less signal decay during SBS. Additionally, enzymes for cleaving the glycoside blocking group may have improved stability during storage and shipping, as they can be freeze dried. Recombinant SBS cleaving enzymes may be produced at scale from relatively low costs, in comparison to, for example, SBS cleavage reagents using precious metal catalysts such as palladium catalysts.

### Definitions

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

Where a range of values is provided, it is understood that the upper and lower limit, and each intervening value between the upper and lower limit of the range is encompassed within the embodiments.

As used herein, common organic abbreviations are defined as follows:
° C. Temperature in degrees Centigrade
dATP Deoxyadenosine triphosphate
dCTP Deoxycytidine triphosphate
dGTP Deoxyguanosine triphosphate
dTTP Deoxythymidine triphosphate
ddNTP Dideoxynucleotide triphosphate
ffN Fully functionalized nucleotide
RT or rt Room temperature
SBS Sequencing by Synthesis
SM Starting material

As used herein, the term “array” refers to a population of different probe molecules that are attached to one or more substrates such that the different probe molecules can be differentiated from each other according to relative location. An array can include different probe molecules that are each located at a different addressable location on a substrate. Alternatively, or additionally, an array can include separate substrates each bearing a different probe molecule, wherein the different probe molecules can be identified according to the locations of the substrates on a surface to which the substrates are attached or according to the locations of the substrates in a liquid. Exemplary arrays in which separate substrates are located on a surface include, without limitation, those including beads in wells as described, for example, in U.S. Pat. No. 6,355,431 B1, US 2002/0102578 and PCT Publication No. WO 00/63437. Exemplary formats that can be used in the invention to distinguish beads in a liquid array, for example, using a microfluidic device, such as a fluorescent activated cell sorter (FACS), are described, for example, in U.S. Pat. No. 6,524,793. Further examples of arrays that can be used in the invention include, without limitation, those described in U.S. Pat. Nos. 5,429,807; 5,436,327; 5,561,071; 5,583,211; 5,658,734; 5,837,858; 5,874,219; 5,919,523; 6,136,269; 6,287,768; 6,287,776; 6,288,220; 6,297,006; 6,291,193; 6,346,413; 6,416,949; 6,482,591; 6,514,751 and 6,610,482; and WO 93/17126; WO 95/11995; WO 95/35505; EP 742 287; and EP 799 897.

As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

As used herein, any “R” group(s) represent substituents that can be attached to the indicated atom. An R group may be substituted or unsubstituted. If two “R” groups are described as “together with the atoms to which they are attached” forming a ring or ring system, it means that the collective unit of the atoms, intervening bonds and the two R groups are the recited ring. For example, when the following substructure is present:

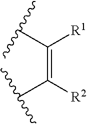

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

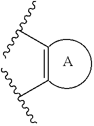

where A is an aryl ring or a carbocyclyl containing the depicted double bond.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “Cₐ to C_{b}” in which “a” and “b” are integers refer to the number of carbon atoms in an alkyl, alkenyl or alkynyl group, or the number of ring atoms of a cycloalkyl or aryl group. That is, the alkyl, the alkenyl, the alkynyl, the ring of the cycloalkyl, and ring of the aryl can contain from “a” to “b”, inclusive, carbon atoms. For example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—; a C₃ to C₄ cycloalkyl group refers to all cycloalkyl groups having from 3 to 4 carbon atoms, that is, cyclopropyl and cyclobutyl. Similarly, a “4 to 6 membered heterocyclyl” group refers to all heterocyclyl groups with 4 to 6 total ring atoms, for example, azetidine, oxetane, oxazoline, pyrrolidine, piperidine, piperazine, morpholine, and the like. If no “a” and “b” are designated with regard to an alkyl, alkenyl, alkynyl, cycloalkyl, or aryl group, the broadest range described in these definitions is to be assumed. As used herein, the term “C₁-C₆” includes C₁, C₂, C3, C₄, C₅ and C₆, and a range defined by any of the two numbers. For example, C₁-C₆ alkyl includes C₁, C₂, C₃, C₄, C₅ and C₆ alkyl, C₂-C₆ alkyl, C₁-C₃ alkyl, etc. Similarly, C₂-C₆ alkenyl includes C₂, C₃, C₄, C₅ and C₆ alkenyl, C₂-C₅ alkenyl, C₃-C₄ alkenyl, etc.; and C₂-C₆ alkynyl includes C₂, C₃, C₄, C₅ and C₆ alkynyl, C₂-C₅ alkynyl, C₃-C₄ alkynyl, etc. C₃-C₈ cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. The alkyl group may be designated as “C₁-C₄ alkyl” or similar designations. By way of example only, “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as “C₁-C₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 6 carbon atoms. The alkenyl group may be designated as “C₂-C₆ alkenyl” or similar designations. By way of example only, “C₂-C₆ alkenyl” indicates that there are two to six carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 6 carbon atoms. The alkynyl group may be designated as “C₂-C₆ alkynyl” or similar designations. By way of example only, “C₂-C₆ alkynyl” indicates that there are two to six carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 6 carbon atoms. The heteroalkyl group may be designated as “C₁-C₆ heteroalkyl” or similar designations. The heteroalkyl group may contain one or more heteroatoms. By way of example only, “C₄-C₆ heteroalkyl” indicates that there are four to six carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆-C₁₀ aryl,” “C₆ or C₁₀ aryl,” or similar designations. Examples of aryl groups include, but are not limited to, phenyl, naphthyl, azulenyl, and anthracenyl.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁-C₆ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinolinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazolylalkyl, and imidazolylalkyl. In some cases, the alkylene group is a lower alkylene group (i.e., a C₁-C₆ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃-C₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “cycloalkyl” means a fully saturated carbocyclyl ring or ring system. Examples include cyclopropyl, cyclobutyl, cyclopentyl, and cyclohexyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidinyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, “alkoxyalkyl” or “(alkoxy)alkyl” refers to an alkoxy group connected via an alkylene group, such as C₂-C₈ alkoxyalkyl, or (C₁-C₆ alkoxy)C₁-C₆ alkyl, for example, —(CH₂)₁₋₃—OCH₃.

As used herein, “—O-alkoxyalkyl” or “—O-(alkoxy)alkyl” refers to an alkoxy group connected via an —O-(alkylene) group, such as —O—(C₁-C₆ alkoxy)C₁-C₆ alkyl, for example, —O—(CH₂)₁₋₃—OCH₃.

As used herein, “(heterocyclyl)alkyl” refer to a heterocyclic or a heterocyclyl group, as defined above, connected, as a substituent, via an alkylene group, as defined above. The alkylene and heterocyclyl groups of a (heterocyclyl)alkyl may be substituted or unsubstituted. Examples include but are not limited to (tetrahydro-2H-pyran-4-yl)methyl, (piperidin-4-yl)ethyl, (piperidin-4-yl)propyl, (tetrahydro-2H-thiopyran-4-yl)methyl, and (1,3-thiazinan-4-yl)methyl.

As used herein, “(cycloalkyl)alkyl” or “(carbocyclyl)alkyl” refers to a cycloalkyl or carbocyclyl group (as defined herein) connected, as a substituent, via an alkylene group. Examples include but are not limited to cyclopropylmethyl, cyclobutylmethyl, cyclopentylethyl, and cyclohexylpropyl.

An “O-carboxy” group refers to a “—OC(═O)R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-carboxy” group refers to a “—C(═O)OR” group in which R is selected from the group consisting of hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein. A non-limiting example includes carboxyl (i.e., —C(═O)OH).

A “sulfonyl” group refers to an “—SO₂R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “sulfino” group refers to a “—S(═O)OH” group.

A “S-sulfonamido” group refers to a “—SO₂NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-sulfonamido” group refers to a “—N(R_{A})SO₂R_{B}” group in which R_{A} and R_{b} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-amido” group refers to a “—C(═O)NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-amido” group refers to a “—N(R_{A})C(═O)R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “amino” group refers to a “—NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein. A non-limiting example includes free amino (i.e., —NH₂).

An “aminoalkyl” group refers to an amino group connected via an alkylene group.

An “(alkoxy)alkyl” group refers to an alkoxy group connected via an alkylene group, such as a “(C₁-C₆ alkoxy) C₁-C₆ alkyl” and the like.

The term “hydroxy” as used herein refers to a —OH group.

The term “cyano” group as used herein refers to a “—CN” group.

The term “azido” as used herein refers to a —N₃ group.

When a group is described as “optionally substituted” it may be either unsubstituted or substituted. Likewise, when a group is described as being “substituted,” the substituent may be selected from one or more of the indicated substituents. As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), (aryl)C₁-C₆ alkyl (optionally substituted with halo, C₁-C₆ alkyl, C₁-C₆ alkoxy, C₁-C₆haloalkyl, and C₁-C₆ haloalkoxy), 5-10 membered heteroaryl (optionally substituted with halo, C₁-C₆alkyl, C₁-C₆ alkoxy, C₁-C₆ haloalkyl, and C₁-C₆ haloalkoxy), (5-10 membered heteroaryl)C₁-C₆ alkyl (optionally substituted with halo, C₁-C₆ alkyl, C₁-C₆ alkoxy, C₁-C₆ haloalkyl, and C₁-C₆ haloalkoxy), halo, —CN, hydroxy, C₁-C₆ alkoxy, (C₁-C₆ alkoxy)C₁-C₆ alkyl, —O(C₁-C₆ alkoxy)C₁-C₆ alkyl; (C₁-C₆ haloalkoxy)C₁-C₆ alkyl; —O(C₁-C₆ haloalkoxy)C₁-C₆ alkyl; aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, 0-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, —SO₃H, sulfino, —OSO₂C₁₋₄alkyl, monophosphate, diphosphate, triphosphate, oxo (═O) and thioxo (═S). Wherever a group is described as “optionally substituted” that group can be substituted with the above substituents.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

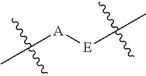

includes the substituent being oriented such that the A is attached at the leftmost attachment point of the molecule as well as the case in which A is attached at the rightmost attachment point of the molecule. In addition, if a group or substituent is depicted as

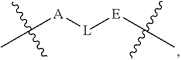

and L is defined an optionally present linker moiety; when L is not present (or absent), such group or substituent is equivalent to

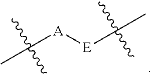

As used herein, a “nucleotide” includes a nitrogen containing heterocyclic base, a sugar, and one or more phosphate groups. They are monomeric units of a nucleic acid sequence. In RNA, the sugar is a ribose, and in DNA a deoxyribose, i.e. a sugar lacking a hydroxyl group that is present in ribose. The nitrogen containing heterocyclic base can be purine or pyrimidine base. Purine bases include adenine (A), deaza adenine (e.g., 7-deaza adenine), guanine (G), deaza guanine (e.g., 7-deaza guanine) and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine.

As used herein, a “nucleoside” is structurally similar to a nucleotide, but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers.

The term “purine base” is used herein in its ordinary sense as understood by those skilled in the art, and includes its tautomers. Similarly, the term “pyrimidine base” is used herein in its ordinary sense as understood by those skilled in the art, and includes its tautomers. A non-limiting list of optionally substituted purine-bases includes purine, deazapurine, adenine, 7-deaza adenine, guanine, 7-deaza guanine, hypoxanthine, xanthine, alloxanthine, 7-alkylguanine (e.g., 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

As used herein, when an oligonucleotide or polynucleotide is described as “comprising” or “labeled with” a nucleoside or nucleotide described herein, it means that the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein forms a covalent bond with the oligonucleotide or polynucleotide. In some such embodiments, the covalent bond is formed between a 3′ hydroxy group of the oligonucleotide or polynucleotide with the 5′ phosphate group of a nucleotide described herein as a phosphodiester bond between the 3′ carbon atom of the oligonucleotide or polynucleotide and the 5′ carbon atom of the nucleotide.

As used herein, the term “cleavable linker” is not meant to imply that the whole linker is required to be removed. The cleavage site can be located at a position on the linker that ensures that part of the linker remains attached to the detectable label and/or nucleoside or nucleotide moiety after cleavage.

As used herein, the term “self-immolative linker” refers to a linker moiety (e.g., a covalent construct) that can degrade spontaneously in response to specific stimuli (e.g., by an enzyme), results in cleavage of two or more chemical bonds.

As used herein, “derivative” or “analog” means a synthetic nucleotide or nucleoside derivative having modified base moieties and/or modified sugar moieties. Such derivatives and analogs are discussed in, e.g., Scheit, Nucleotide Analogs (John Wiley & Son, 1980) and Uhlman et al., Chemical Reviews 90:543-584, 1990. Nucleotide analogs can also comprise modified phosphodiester linkages, including phosphorothioate, phosphorodithioate, alkyl-phosphonate, phosphoranilidate and phosphoramidate linkages. “Derivative,” “analog,” and “modified” as used herein, may be used interchangeably, and are encompassed by the terms “nucleotide” and “nucleoside” defined herein.

As used herein, the term “phosphate” is used in its ordinary sense as understood by those skilled in the art, and includes its protonated forms

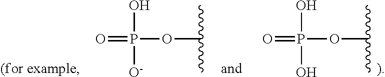 [SMILES: *OP(=O)([O-])O ; *OP(=O)(O)O]

As used herein, the terms “monophosphate,” “diphosphate,” and “triphosphate” are used in their ordinary sense as understood by those skilled in the art, and include protonated forms.

The terms “protecting group” and “protecting groups” as used herein refer to any atom or group of atoms that is added to a molecule in order to prevent existing groups in the molecule from undergoing unwanted chemical reactions. Sometimes, “protecting group” and “blocking group” can be used interchangeably.

As used herein, the term “phasing” refers to a phenomenon in SBS that is caused by incomplete removal of the 3′ terminators and fluorophores, and failure to complete the incorporation of a portion of DNA strands within clusters by polymerases at a given sequencing cycle. Pre-phasing is caused by the incorporation of nucleotides without effective 3′ terminators, wherein the incorporation event goes 1 cycle ahead due to a termination failure. Phasing and pre-phasing cause the measured signal intensities for a specific cycle to consist of the signal from the current cycle as well as noise from the preceding and following cycles. As the number of cycles increases, the fraction of sequences per cluster affected by phasing and pre-phasing increases, hampering the identification of the correct base. Pre-phasing can be caused by the presence of a trace amount of unprotected or unblocked 3′-OH nucleotides during sequencing by synthesis (SBS). The unprotected 3′-OH nucleotides could be generated during the manufacturing processes or possibly during the storage and reagent handling processes. Accordingly, the discovery of nucleotide analogues which decrease the incidence of pre-phasing is surprising and provides a great advantage in SBS applications over existing nucleotide analogues. For example, the nucleotide analogues provided can result in faster SBS cycle time, lower phasing and pre-phasing values, and longer sequencing read lengths.

Nucleosides and Nucleotides with 3′-O-Glycoside Blocking Groups

One aspect of the present disclosure relates to a nucleotide comprising a ribose or 2′ deoxyribose having an enzymatically removable 3′ blocking group in the form of a 3′-O-glycoside group (3′-O-glycosidyl), a nucleobase and a triphosphate moiety, wherein the 3′ blocking group forms an —O-glycosidic bond with the 3′ carbon atom of the nucleotide. In some embodiments, the glycoside has a 5 or 6 membered monocyclic furanose or pyranose ring. In some embodiments, the —O-glycosidic bond can be formed by reacting the glycoside moiety with the 3′-hydroxy group of the nucleotide.

In some embodiments, the 3′-O-glycoside blocking group has the structure:

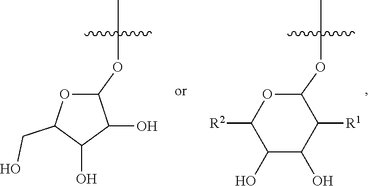

wherein the squiggle line indicates the point of attachment to the 3′-carbon atom, wherein:
R¹ is OH or —NHR³;
R² is H, —CH₂OH, or —C(═O)OR⁴;
R³ is H or an amino protecting group; and
R⁴ is H or C₁-C₆ alkyl. In some such embodiments, the removable 3′ blocking group has a structure:

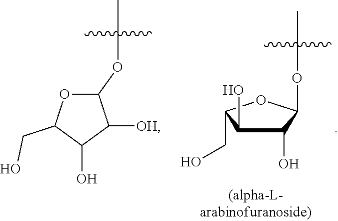

or beta-L-arabinofuranoside. In some other embodiments, the removable 3′ blocking group has a structure

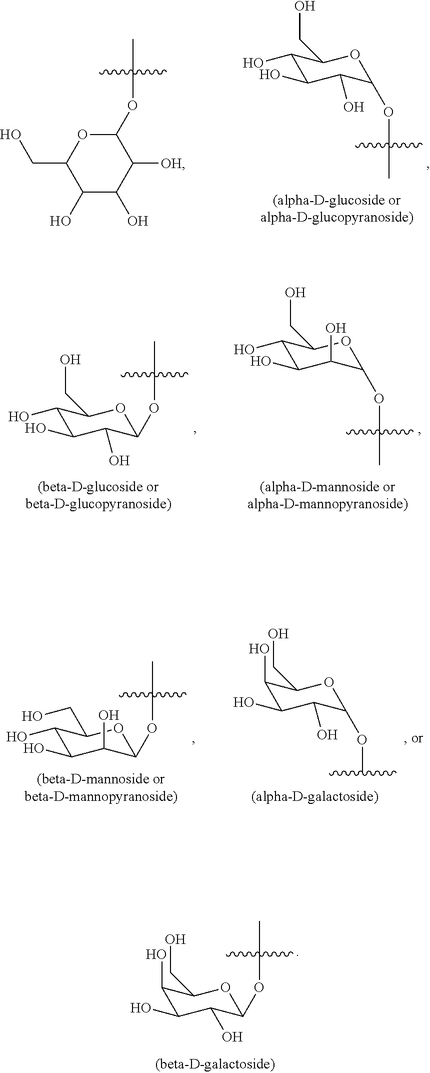

In some other embodiments, the removable 3′ blocking group has a structure

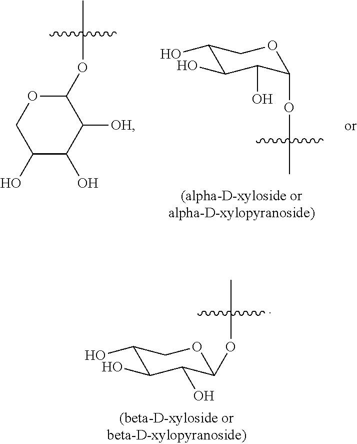 [SMILES: *OC1OCC(O)C(O)C1O ; *OC1OCC(O)C(O)C1O ; *OC1OCC(O)C(O)C1O]

In some other embodiments, the removable 3′ blocking group has a structure

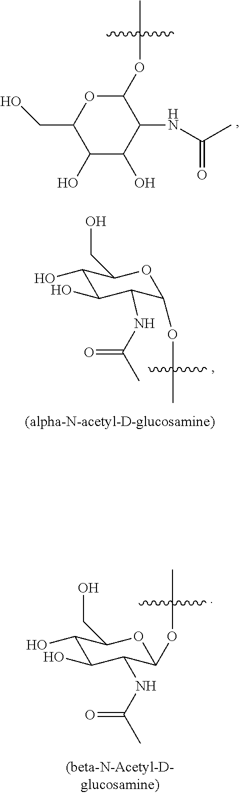

In some embodiments, the removable 3′ blocking group has a structure

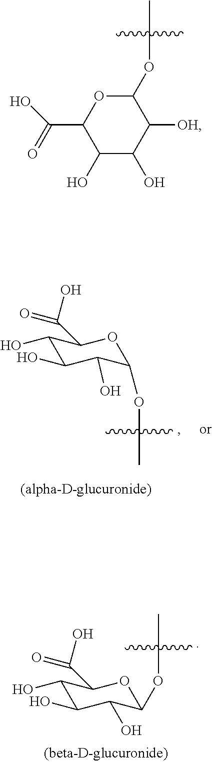 [SMILES: *OC1OC(C(=O)O)C(O)C(O)C1O ; *OC1OC(C(=O)O)C(O)C(O)C1O ; *OC1OC(C(=O)O)C(O)C(O)C1O]

In addition to the nucleotides described herein, nucleosides, nucleoside monophosphate, nucleoside diphosphate, and nucleoside thiophosphates (in which one or more O is replaced by S in the mono-, di- or tri-phosphate groups) with the 3′-O-glycoside blocking groups are also intended.

In some embodiments, the nucleotide further comprises a detectable label. In further embodiments, the detectable label is a fluorescent dye. In further embodiments, the detectable label is connected to the nucleobase via a cleavable linker. In yet further embodiments, the cleavable linker is enzymatically cleavable. In further embodiments, the cleavable linker is an enzyme-triggered self-immolative linker. In further embodiments, the detectable label and the 3′ blocking group are removable by a single enzymatic reaction.

Other aspects of the present disclosure relate to an oligonucleotide or polynucleotide comprising a nucleotide in accordance with the present disclosure. In some embodiments, the oligonucleotide or polynucleotide is at least partially complementary and hybridized to a target polynucleotide immobilized on a surface of a solid support. In further embodiments, the solid support comprises an array of a plurality of target polynucleotides immobilized thereon. Additional embodiments of the present disclosure relate to a solid support comprising an array of a plurality of immobilized template or target polynucleotides and at least a portion of such immobilized template or target polynucleotides is hybridized to an oligonucleotide or a polynucleotide comprising a nucleotide described herein.

### Enzymes Capable of Cleaving 3′-O-Glycoside Blocking Groups

Certain enzymes may be capable of cleaving 3′-O-glycoside blocking groups described herein. For example, glycoside hydrolases or glycosyl hydrolases may be suitable for cleaving 3′-O-glycoside blocking groups in accordance with the present disclosure. Table 1 lists particular example glycoside hydrolases that can cleave 3′-O-glycoside blocking groups. Enzymes in accordance with the present disclosure may be suitable for inclusion in a kit for sequencing by synthesis. Enzymes in accordance with the present disclosure may be suitable for use in methods of sequencing. Enzymes in accordance with the present disclosure may be suitable in methods of growing a polynucleotide strand.

TABLE 1
Exemplary enzymes and corresponding E.C. Number
and Carbohydrate Active enZyme (CAZy) family

| | | CAZy Family |
|---|---|---|
| Enzyme Name | E.C. Number | (as of July 2023) |
| α-L-arabinofuranosidase | E.C. 3.2.1.55 | GH 2, 3, 10, 43, |
| | | 51, 54, 62 and 159 |
| β-L-arabinofuranosidase | E.C. 3.2.1.185 | GH 127, 142, 143 |
| | | and 146 |
| α-D-glucosidase | E.C. 3.2.1.20 | GH 4, 13, 31, 76, |
| | | 97 and 122 |
| β-D-glucosidase | E.C. 3.2.1.21 | GH 1, 2, 3, 5, 16, |
| | | 30, 39, 116, 131, |
| | | 175 and 180 |
| α-D-mannosidase | E.C. 3.2.1.24 | GH 38 and 92 |
| β-D-mannosidase | E.C. 3.2.1.25 | GH 1, 2, 5, 113 and |
| | | 164 |
| α-D-xylosidase | E.C. 3.2.1.177 | GH 31 |
| β-D-xylosidase | E.C. 3.2.1.37 | GH 1, 2, 3, 5, 8, |
| | | 10, 30, 39, 43, |
| | | 51, 52, 54 and 120 |
| α-D-galactosidase | E.C. 3.2.1.22 | GH 4, 27, 31, 36, |
| | | 57, 97 and 110 |
| β-D-galactosidase | E.C. 3.2.1.23 | GH 1, 2, 5, 16, 35, |
| | | 42, 50, 54, 147, |
| | | 165 and 173 |
| N-Acetyl-α-D-glucosaminidase | E.C. 3.2.1.50 | GH 89 |
| β-N-acetylhexosaminidase | E.C. 3.2.1.52 | GH 2, 3, 18, 20, |
| | | 84 and 179 |
| β-D-glucuronidase | E.C. 3.2.1.31 | GH 1, 2, 3, 79 and 137 |
| α-D-glucuronidase | E.C. 3.2.1.139 | GH 67 |

(GH = Glycoside or Glycosyl Hydrolase)

### Labeled Nucleotides

According to an aspect of the disclosure, the described 3′-OH blocked nucleotide also comprises a detectable label. Such a nucleotide is referred to herein as a “labeled nucleotide.” The label (e.g., a fluorescent dye) can be conjugated via an optional linker by a variety of means including hydrophobic attraction, ionic attraction, and covalent attachment. In some aspect, the detectable label is conjugated to the substrate by covalent attachment. More particularly, the covalent attachment is by means of a linker group. In some instances, such labeled nucleotides are also referred to as “modified nucleotides.”

Various fluorescent dyes may be used in the present disclosure as detectable labels, in particularly those dyes that may be excitation by a blue light (e.g., about 450 nm to about 460 nm) or a green light (e.g., about 520 nm to about 540 nm). These dyes may also be referred to as “blue dyes” and “green dyes” respectively. Examples of various type of blue dyes, including but not limited to coumarin dyes, chromenoquinoline dyes, and bisboron containing heterocycles are disclosed in U.S. Publication Nos. 2018/0094140, 2018/0201981, 2020/0277529, 2020/0277670, 2021/0188832, 2022/0033900, 2022/0195517, 2022/0380389, and 2023/0313292, and 2023/0416279, each of which is incorporated by reference in its entirety. Examples of green dyes including cyanine or polymethine dyes disclosed in International Publication Nos. WO2013/041117, WO2014/135221, WO 2016/189287, WO2017/051201 and WO2018/060482A1, each of which is incorporated by reference in its entirety.

Labeled nucleosides and nucleotides are useful for labeling polynucleotides formed by enzymatic synthesis, such as, by way of non-limiting example, in PCR amplification, isothermal amplification, solid phase amplification, polynucleotide sequencing (e.g., solid phase sequencing), nick translation reactions and the like.

In some embodiments, the dye may be covalently attached to oligonucleotides or nucleotides via the nucleotide base. For example, the labeled nucleotide or oligonucleotide may have the label attached to the C5 position of a pyrimidine base or the C7 position of a 7-deaza purine base through a linker moiety.

Unless indicated otherwise, the reference to nucleotides is also intended to be applicable to nucleosides. The present application will also be further described with reference to DNA, although the description will also be applicable to RNA, PNA, and other nucleic acids, unless otherwise indicated.

### Linkers

In some embodiments described herein, the purine or pyrimidine base of the nucleotide or nucleoside molecules described herein can be linked to a detectable label as described above. In some such embodiments, the linkers used are cleavable. The use of a cleavable linker ensures that the label can, if required, be removed after detection, avoiding any interfering signal with any labeled nucleotide or nucleoside incorporated subsequently. In some embodiments, the cleavable linker comprises an azido moiety, a —O—C₂-C₆ alkenyl moiety (e.g., —O-allyl), a disulfide moiety, an acetal moiety (same or similar to the 3′acetal blocking group described herein), or a thiocarbamate moiety (same or similar to the 3′acetal blocking group described herein).

In some other embodiments, the linkers used are non-cleavable. Since in each instance where a labeled nucleotide of the invention is incorporated, no nucleotides need to be subsequently incorporated and thus the label need not be removed from the nucleotide.

In some embodiments in accordance with the present disclosure, the linker group may be enzymatically cleavable. In some further embodiments, the linker group may be enzymatically cleavable by the same enzyme that can cleave the 3′-O-glycoside blocking group in accordance with the present disclosure. In other embodiments, the linker group may be enzymatically cleavable by a different enzyme than the enzyme capable of cleaving the 3′-O-glycoside blocking group.

Cleavable linkers are known in the art, and conventional chemistry can be applied to attach a linker to a nucleotide base and a label. The linker can be cleaved by any suitable method, including exposure to acids, bases, nucleophiles, electrophiles, radicals, metals, reducing or oxidizing agents, light, temperature, enzymes etc. The linker as discussed herein may also (or alternatively) be cleaved using an enzyme. In some examples, the same enzymatic catalyst used to cleave the 3′-O-blocking group bond can be used to cleave the linker. In some further embodiments, the linker group may be enzymatically cleavable by the same enzyme that can cleave the 3′-O-glycoside blocking group in accordance with the present disclosure. In other embodiments, the linker group may be enzymatically cleavable by a different enzyme than the enzyme capable of cleaving the 3′-O-glycoside blocking group. Suitable linkers can be adapted from standard chemical protecting groups, as disclosed in Greene & Wuts, Protective Groups in Organic Synthesis, John Wiley & Sons. Further suitable cleavable linkers used in solid-phase synthesis are disclosed in Guillier et al. (Chem. Rev. 100:2092-2157, 2000).

Where the detectable label is attached to the base, the linker can be attached at any position on the nucleotide base provided that Watson-Crick base pairing can still be carried out. In the context of purine bases, it is preferred if the linker is attached via the 7-position of the purine or the preferred deazapurine analogue, via an 8-modified purine, via an N-6 modified adenosine or an N-2 modified guanine. For pyrimidines, attachment is preferably via the 5-position on cytosine, thymidine or uracil and the N-4 position on cytosine.

In some embodiments, the linker can comprise a spacer unit. The length of the linker is unimportant provided that the label is held a sufficient distance from the nucleotide so as not to interfere with any interaction between the nucleotide and an enzyme, for example, a polymerase.

In some embodiments, the linker may consist of the similar functionality as the 3′-OH protecting group. This will make the deprotection and deprotecting process more efficient, as only a single treatment will be required to remove both the label and the protecting group.

Use of the term “cleavable linker” is not meant to imply that the whole linker is required to be removed. The cleavage site can be located at a position on the linker that ensures that part of the linker remains attached to the dye and/or substrate moiety after cleavage. Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

Useful linker groups may be found in PCT Publication No. WO2004/018493 (herein incorporated by reference), examples of which include linkers that may be cleaved using water-soluble phosphines or water-soluble transition metal catalysts formed from a transition metal and at least partially water-soluble ligands, for example, a Pd(II) complex and THP. In aqueous solution the latter form at least partially water-soluble transition metal complexes. Such cleavable linkers can be used to connect bases of nucleotides to labels such as the dyes set forth herein.

Particular linkers include those disclosed in PCT Publication No. WO2004/018493 (herein incorporated by reference) such as those that include moieties of the formulae:

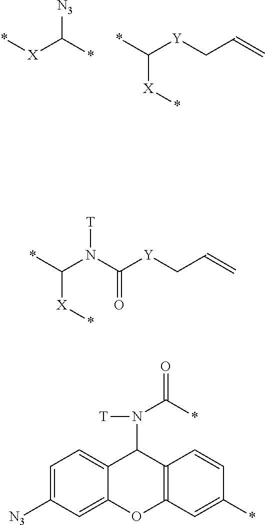

(wherein X is selected from the group comprising O, S, NH and NQ wherein Q is a C₁₋₁₀ substituted or unsubstituted alkyl group, Y is selected from the group comprising O, S, NH and N(allyl), T is hydrogen or a C₁-C₁₀ substituted or unsubstituted alkyl group and * indicates where the moiety is connected to the remainder of the nucleotide or nucleoside). In some aspect, the linkers connect the bases of nucleotides to labels such as, for example, the dye compounds described herein.

Additional examples of linkers include those disclosed in U.S. Publication No. 2016/0040225 (herein incorporated by reference), such as those include moieties of the formulae:

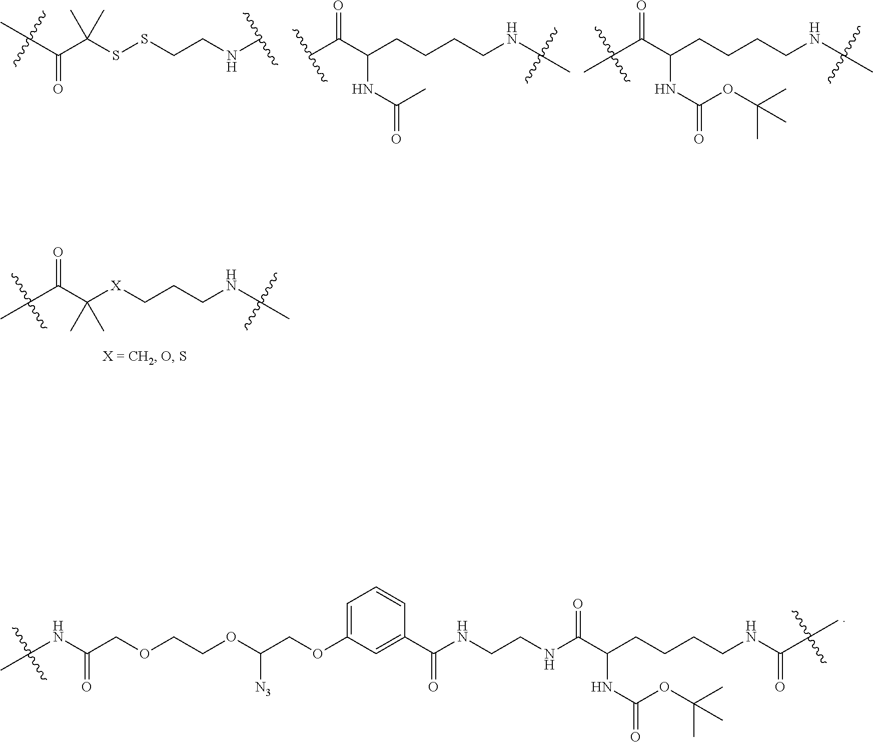

The linker moieties illustrated herein may comprise the whole or partial linker structure between the nucleotides/nucleosides and the labels.

Additional examples of linkers are disclosed in U.S. Publication No. 2020/0216891 A1, which is incorporated by reference in its entirety:

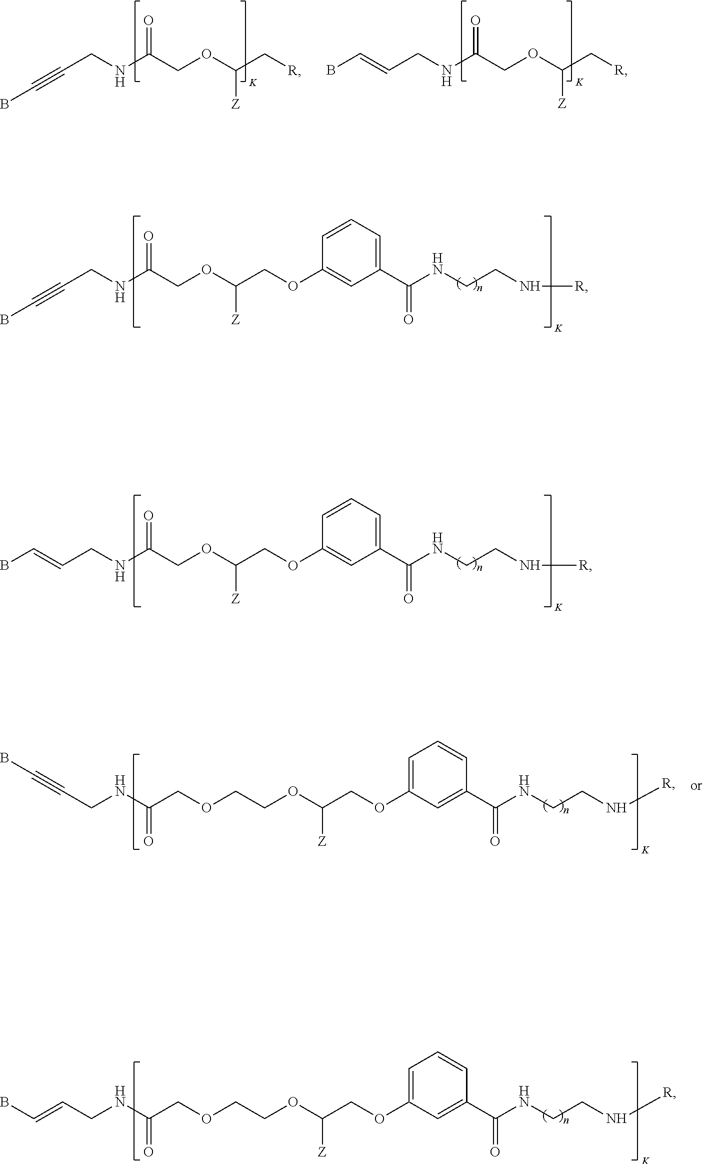

wherein B is a nucleobase; n is 1, 2, 3, 4, 5; k is 1; Z is —N₃ (azido), —O—C₁-C₆ alkyl, —O—C₂-C₆ alkenyl, or —O—C₂-C₆ alkynyl; and R comprises a detectable label or a binding moiety for post-incorporation labeling, which may contain additional linker and/or spacer structure. One of ordinary skill in the art understands that the detectable label or the binding moiety described herein is covalently bound to the linker by reacting a functional group of the binding moiety containing compound (e.g., carboxyl) with a functional group of the linker (e.g., amino) to form an amide bond. In one embodiment, the cleavable linker comprises

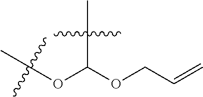 [SMILES: *OC(*)OCC=C]

(“AOL” linker moiety) where Z is —O-allyl. For the purpose of the present disclosure, the nucleotide may contain multiple cleavable linkers repeating units (e.g., k is 1, 2, 3, 4 5, 6, 7, 8, 9 or 10). the linker can also comprise a spacer unit, such as one or more PEG unit(s) (—OCH₂CH₂—)ₙ, where n is an integer of 1-20, for example 1, 2, 3, 4, 5, 6, 7, 8, 9, 10, 11, 12, 13, 14 or 15. The spacer distances, for example, the nucleotide base from a cleavage site or label.

In particular embodiments, the length of the linker between a fluorescent dye (fluorophore) and a guanine base can be altered, for example, by introducing a polyethylene glycol spacer group, thereby increasing the fluorescence intensity compared to the same fluorophore attached to the guanine base through other linkages known in the art. Exemplary linkers and their properties are set forth in PCT Publication No. WO 2007/020457 (herein incorporated by reference). The design of linkers, and especially their increased length, can allow improvements in the brightness of fluorophores attached to the guanine bases of guanosine nucleotides when incorporated into polynucleotides such as DNA. Thus, when the dye is for use in any method of analysis which requires detection of a fluorescent dye label attached to a guanine-containing nucleotide, it is advantageous if the linker comprises a spacer group of formula —((CH₂)₂O)ₙ—, wherein n is an integer between 2 and 50, as described in WO 2007/020457.

Nucleosides and nucleotides may be labeled at sites on the sugar or nucleobase. As known in the art, a “nucleotide” consists of a nitrogenous base, a sugar, and one or more phosphate groups. In RNA, the sugar is ribose and in DNA is a deoxyribose, i.e., a sugar lacking a hydroxy group that is present in ribose. The nitrogenous base is a derivative of purine or pyrimidine. The purines are adenine (A) and guanine (G), and the pyrimidines are cytosine (C) and thymine (T) or in the context of RNA, uracil (U). The C-1 atom of deoxyribose is bonded to N-1 of a pyrimidine or N-9 of a purine. A nucleotide is also a phosphate ester of a nucleoside, with esterification occurring on the hydroxy group attached to the C-3 or C-5 of the sugar. Nucleotides are usually mono-, di- or triphosphates.

A “nucleoside” is structurally similar to a nucleotide but is missing the phosphate moieties. An example of a nucleoside analog would be one in which the label is linked to the base and there is no phosphate group attached to the sugar molecule.

Although the base is usually referred to as a purine or pyrimidine, the skilled person will appreciate that derivatives and analogues are available which do not alter the capability of the nucleotide or nucleoside to undergo Watson-Crick base pairing. “Derivative” or “analogue” means a compound or molecule whose core structure is the same as, or closely resembles that of a parent compound but which has a chemical or physical modification, such as, for example, a different or additional side group, which allows the derivative nucleotide or nucleoside to be linked to another molecule. For example, the base may be a deazapurine. In particular embodiments, the derivatives should be capable of undergoing Watson-Crick pairing. “Derivative” and “analogue” also include, for example, a synthetic nucleotide or nucleoside derivative having modified base moieties and/or modified sugar moieties. Such derivatives and analogues are discussed in, for example, Scheit, Nucleotide analogs (John Wiley & Son, 1980) and Uhlman et al., Chemical Reviews 90:543-584, 1990. Nucleotide analogues can also comprise modified phosphodiester linkages including phosphorothioate, phosphorodithioate, alkyl-phosphonate, phosphoranilidate, phosphoramidate linkages and the like.

A dye may be attached to any position on the nucleotide base, for example, through a linker. In particular embodiments, Watson-Crick base pairing can still be carried out for the resulting analog. Particular nucleobase labeling sites include the C5 position of a pyrimidine base or the C7 position of a 7-deaza purine base. As described above a linker group may be used to covalently attach a dye to the nucleoside or nucleotide.

In particular embodiments the labeled nucleoside or nucleotide may be enzymatically incorporable and enzymatically extendable. Accordingly, a linker moiety may be of sufficient length to connect the nucleotide to the compound such that the compound does not significantly interfere with the overall binding and recognition of the nucleotide by a nucleic acid replication enzyme. Thus, the linker can also comprise a spacer unit. The spacer distances, for example, the nucleotide base from a cleavage site or label.

Nucleosides or nucleotides labeled with the dyes described herein may have the formula:

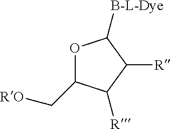

where Dye is a dye compound; B is a nucleobase, such as, for example uracil, thymine, cytosine, adenine (e.g., 7-deaza adenine), guanine (e.g., 7-deaza guanine) and the like; L is an optional linker group which may or may not be present; R′ can be H, or —OR′ is monophosphate, diphosphate, triphosphate, thiophosphate, a phosphate ester analog, —O— attached to a reactive phosphorous containing group, or —O— protected by a blocking group; R′″ can be H, OH, a phosphoramidite, or a 3′-O-glycosidyl blocking group described herein, and R″ is H or OH. Where R′″ is phosphoramidite, R′ is an acid-cleavable hydroxy protecting group which allows subsequent monomer coupling under automated synthesis conditions.

In a particular embodiment, the linker (between dye and nucleotide) and blocking group are both present and are separate moieties. In particular embodiments, the linker and blocking group are both cleavable under substantially similar conditions. Thus, deprotection and deblocking processes may be more efficient because only a single treatment will be required to remove both the dye compound and the blocking group. However, in some embodiments a linker and blocking group need not be cleavable under similar conditions, instead being individually cleavable under distinct conditions.

The disclosure also encompasses polynucleotides incorporating dye compounds. Such polynucleotides may be DNA or RNA comprised respectively of deoxyribonucleotides or ribonucleotides joined in phosphodiester linkage. Polynucleotides may comprise naturally occurring nucleotides, non-naturally occurring (or modified) nucleotides other than the labeled nucleotides described herein or any combination thereof, in combination with at least one modified nucleotide (e.g., labeled with a dye compound) as set forth herein. Polynucleotides according to the disclosure may also include non-natural backbone linkages and/or non-nucleotide chemical modifications. Chimeric structures comprised of mixtures of ribonucleotides and deoxyribonucleotides comprising at least one labeled nucleotide are also contemplated.

Non-limiting exemplary labeled nucleotides as described herein include:

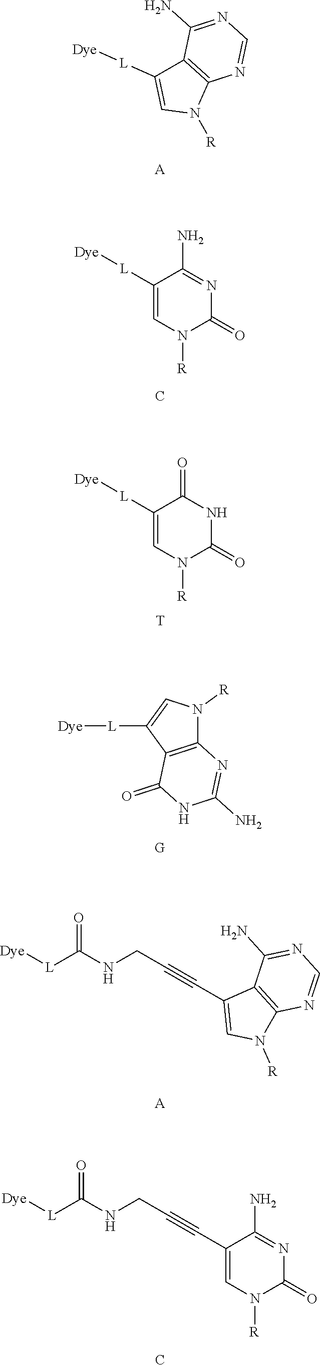

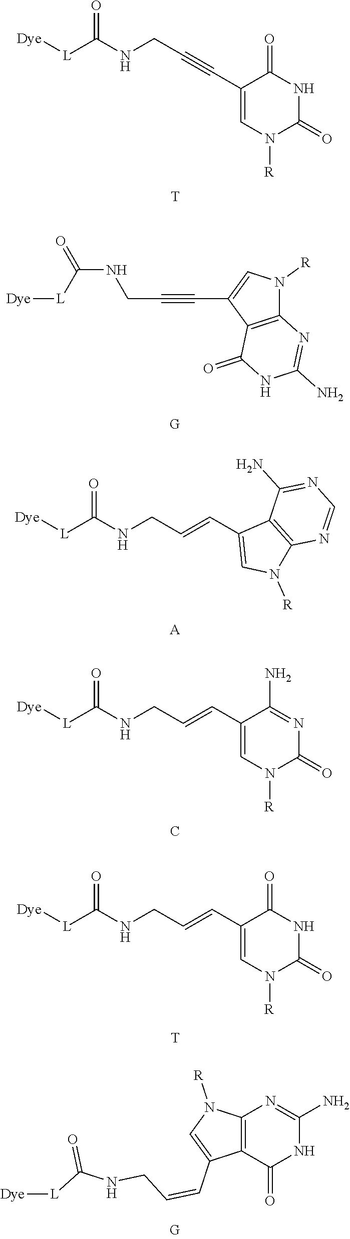

wherein L represents a linker and R represents a sugar residue as described above, or a sugar residue with the 5′ position substituted with one, two or three phosphates.

In some embodiments, non-limiting exemplary fluorescent dye conjugates are shown below:

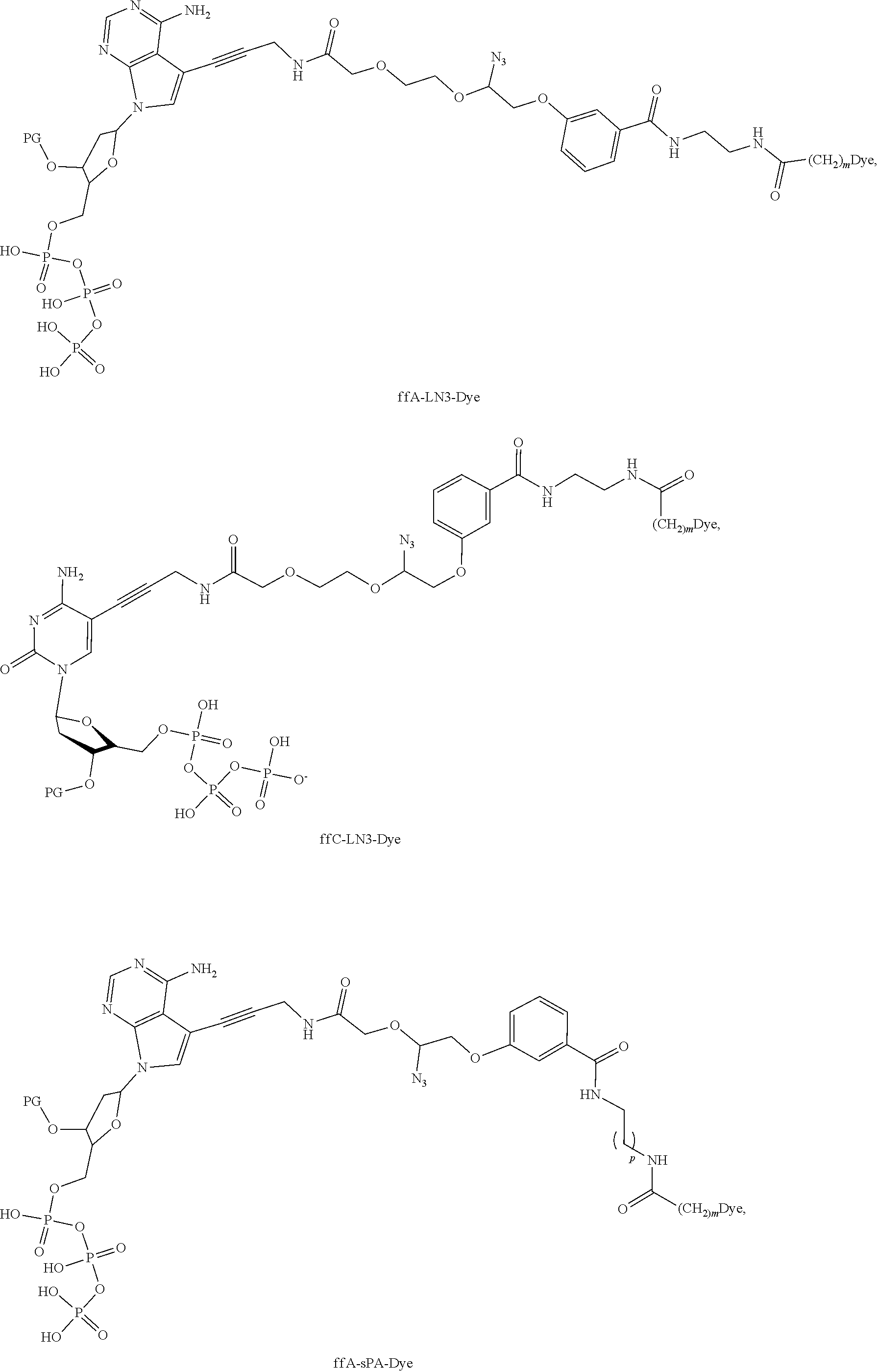

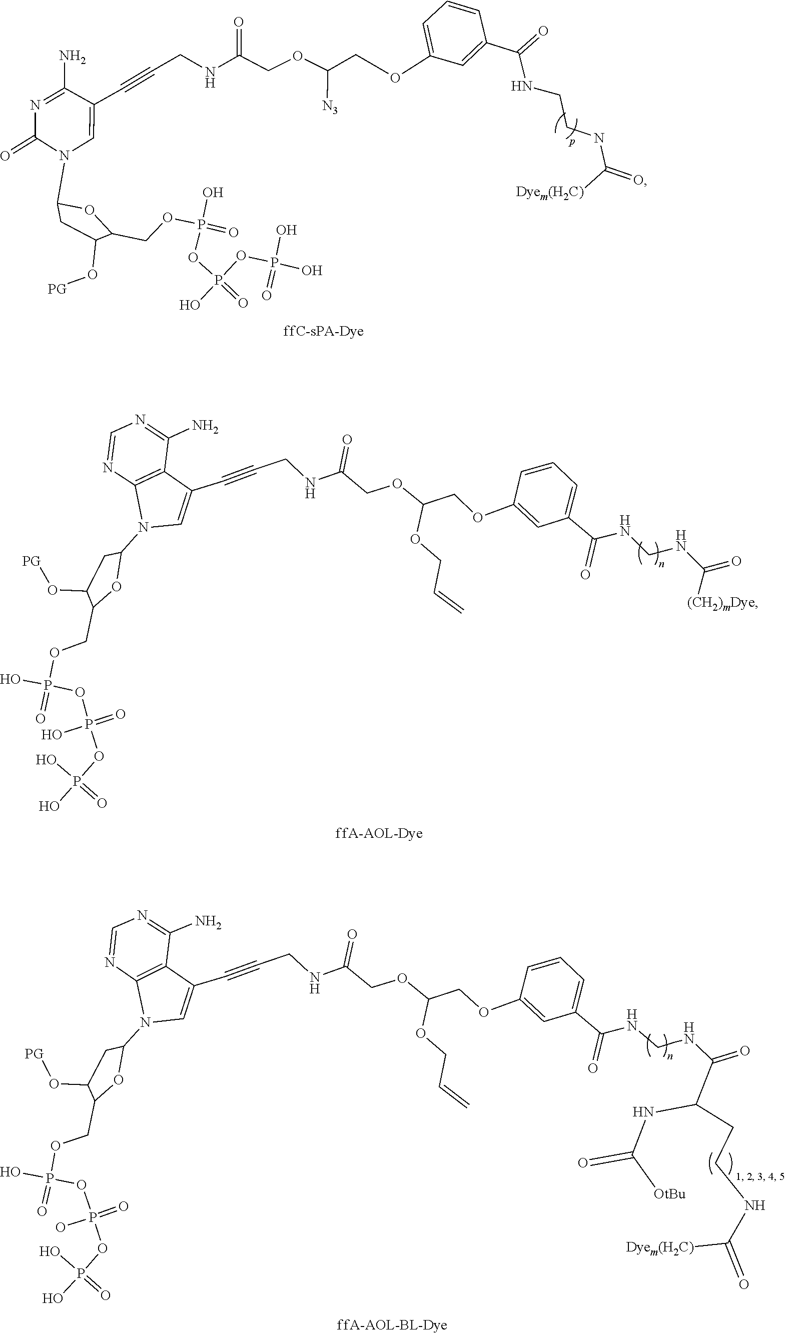

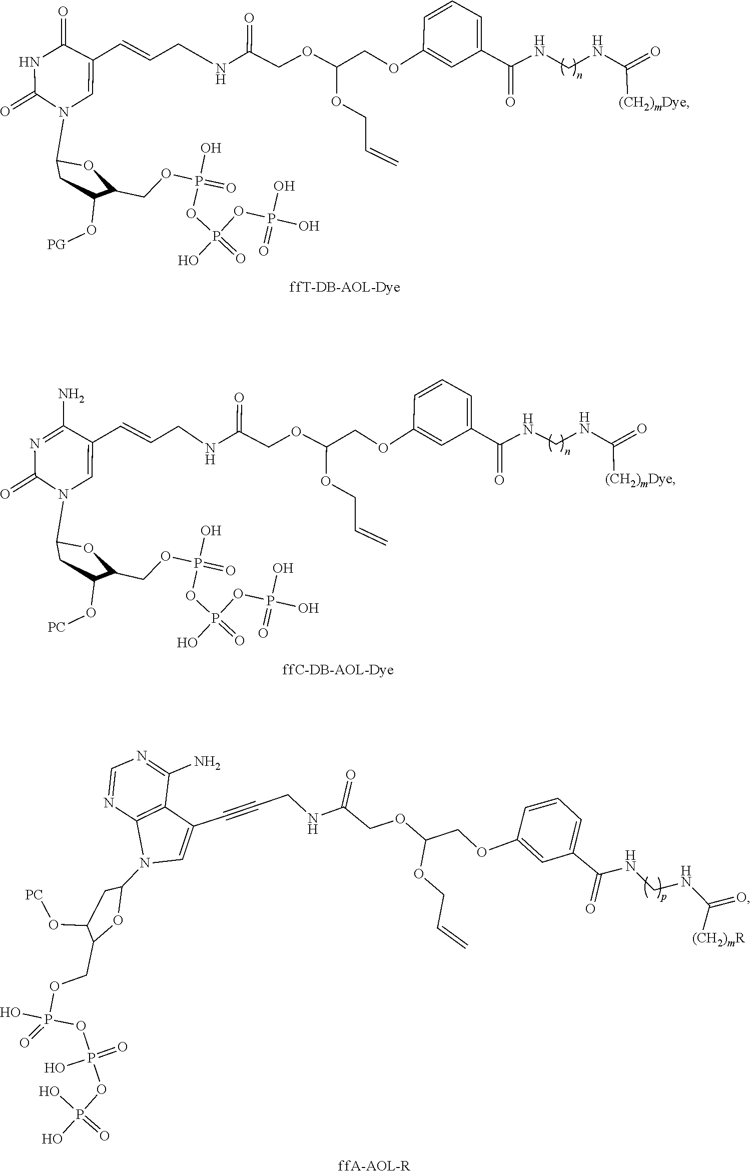

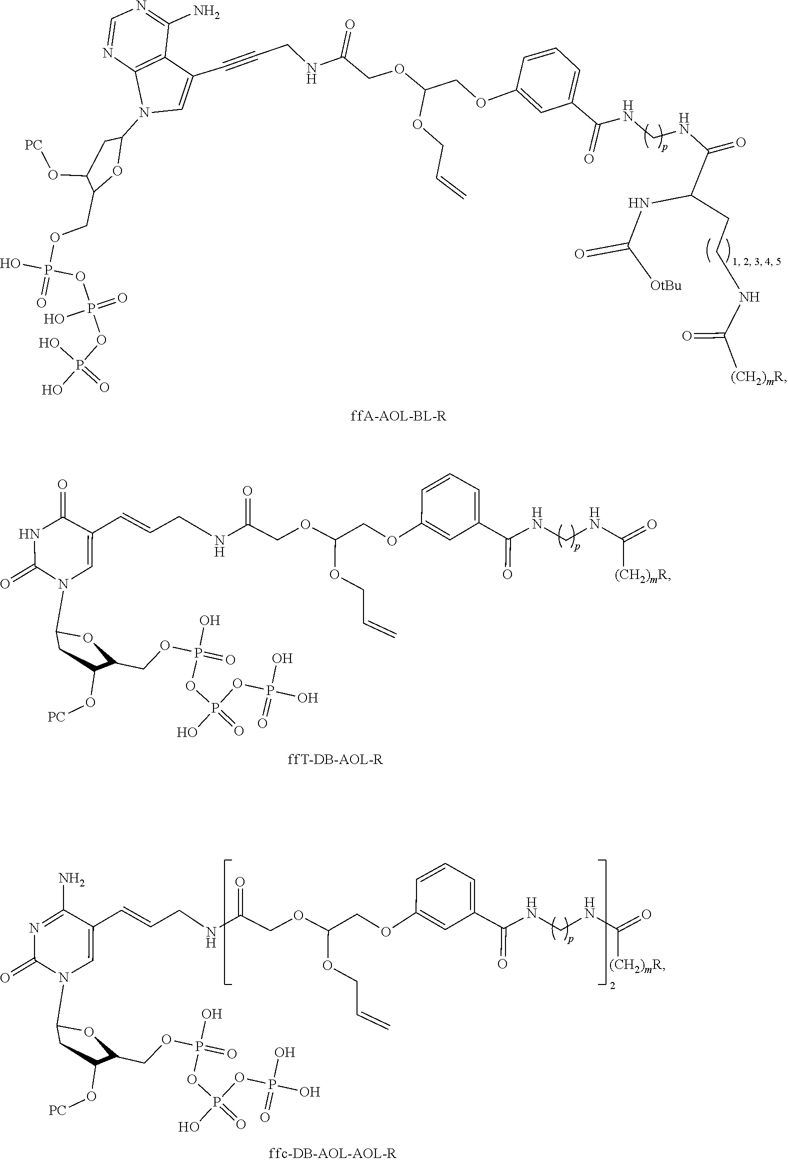

wherein PG stands for the 3′-O-glycosidyl blocking group described herein; each of n and p is an integer of 1, 2, 3, 4, 5, 6, 7, 8, 9, or 10; and m is 0, 1, 2, 3, 4, or 5. In one embodiment, n is 5.

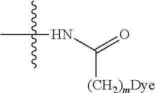

refers to the connection point of the Dye with the cleavable linker as a result of a reaction between an amino group of the linker moiety and the carboxyl group of the Dye. In one embodiment, p is 5.

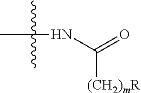

refers to the connection point of a functional moiety of the nucleotide with the cleavable linker as a result of a reaction between an amino group of the linker moiety and the carboxyl group of the functional moiety. In further embodiments, the nucleotide may be attached to the functional moiety via more than one of the same cleavable linkers (such as AOL-AOL). In some embodiments m is 5. In addition, the linker may further include additional PEG spacers as described herein, for example, between R and —(CH₂)ₘ—.

### Methods of Sequencing

One aspect of the present disclosure relates to a method of preparing a growing polynucleotide complementary to a target single-stranded polynucleotide in a sequencing reaction, comprising incorporating a nucleotide in accordance with the present disclosure into a growing complementary polynucleotide, wherein the incorporation of the nucleotide prevents the introduction of any subsequent nucleotide into the growing complementary polynucleotide. In some embodiments, the incorporation of the nucleotide is accomplished by a polymerase, a terminal deoxynucleotidyl transferase, or a reverse transcriptase.

Another aspect of the present disclosure relates to a method of determining the sequences of a plurality of target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an aqueous solution (e.g., an incorporation mixture) comprising DNA polymerase and one or more of four different types of nucleotides (A, G, C, and T or U; dATP, dGTP, dCTP and dTTP or dUTP) under conditions suitable for DNA polymerase-mediated primer extension, wherein each type of nucleotides has a 3′ blocking group and at least one type of nucleotide is a nucleotide with the 3′-O-glycoside blocking group, and wherein each of the one or more of four different type of nucleotides comprises a 2′ deoxyribose;
(c) incorporating one type of nucleotides into the sequencing primers to produce extended copy polynucleotides; and
(d) imaging the solid support and performing one or more fluorescent measurements of the extended copy polynucleotides (i.e., to determine the identity of incorporated nucleotides); and
(e) removing the 3′ blocking group from the nucleotides incorporated into the extended copy polynucleotides.

In some embodiments, step (d) comprises imaging the solid support and performing one or more fluorescent measurements of the extended copy polynucleotides to determine the identity of the incorporated nucleotides. In some embodiments, the method further comprises: (f) washing the solid support after the removal of the 3′ blocking group from the incorporated nucleotides. In some embodiments, the method further comprises repeating steps (b) to (f) until the sequences of at least a portion of the target polynucleotides are determined. In some further embodiments, steps (b) to (f) are repeated at least 50, 100, 150, 200, 250, 300, 350, 400, 450 or 500 cycles. In some embodiments, the detectable label from the nucleotide incorporated into the copy polynucleotide strand is removed under the same reaction condition as the removal of the 3′ blocking group of the nucleotides.

### Post-Incorporation Labeling SBS Workflow

Another aspect of the present disclosure relates to an alternative sequencing by synthesis method in which at least one labeling reagent is introduced after the incorporation of unlabeled nucleotides. In particular, the disclosure relates to a method of determining the sequence of a plurality of different target polynucleotides in parallel, the method comprising:
(a′) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b′) contacting the solid support with an aqueous incorporation mixture comprising DNA polymerase and one or more of four types of nucleotides A, G, C, and T or U; dATP, dGTP, dCTP and dTTP or dUTP) under conditions suitable for DNA polymerase-mediated primer extension, wherein:
   (i) the nucleotides each comprises a 2′ deoxyribose moiety with a 3′ blocking group, and at least one type of nucleotide is a nucleotide with the 3′-O-glycoside blocking group;
   (ii) at least one type of nucleotide is unlabeled; and
   (iii) the first type of unlabeled nucleotides comprises a first functional moiety;
(c′) incorporating one type of nucleotides into the sequencing primers to produce extended copy polynucleotides;
(d′) contacting the extended copy polynucleotides with an aqueous labeling mixture comprising a first labeling reagent, wherein the first labeling reagent comprises one or more first detectable labels and a first binding moiety that is capable of specific binding to the first functional moiety of the first type of unlabeled nucleotide;
(e′) imaging the solid support and performing one or more fluorescent measurements (i.e., to determine the identity of incorporated nucleotides); and
(f′) removing the 3′ blocking group from the nucleotides incorporated into the extended copy polynucleotides.

In some embodiments of the post-incorporation labeling sequencing method described herein, the method further comprises: (g′) washing the solid support after the removal of the 3′ blocking group from the incorporated nucleotides. In some embodiments, the method further comprises repeating steps (b′) to (g′) until the sequences of at least a portion of the target polynucleotides are determined. In some further embodiments, steps (b′) to (g′) are repeated at least 50, 100, 150, 200, 250, 300, 350, 400, 450 or 500 cycles. In some embodiments, each type of nucleotides has a 3′-O-glycoside blocking group as described herein. In some embodiments, at least two types of nucleotides are unlabeled.

In some embodiments, step (c′) is performed under conditions where the first labeling reagent binds specifically to the incorporated unlabeled first type of nucleotides to provide labeled extended copy polynucleotides. In some embodiments, the first functional moiety of the first type of unlabeled nucleotide is bound to the first labeling reagent by covalent bonding, optionally via the cleavable linker as described herein. In some other embodiments, the first functional moiety of the first type of unlabeled nucleotide is bound to the first labeling reagent by noncovalent interaction, optionally via the cleavable linker as described herein.

In some embodiments, each of the four types of nucleotides in the aqueous incorporation mixture is unlabeled, the second type of unlabeled nucleotides comprises a second functional moiety, wherein the aqueous labeling mixture comprises a second labeling reagent, and the second labeling reagent comprises one or more second detectable labels and a second binding moiety that is capable of specific binding to the second functional moiety of the second type of unlabeled nucleotides. In some such embodiments, step (c′) is performed under conditions where the first labeling reagent binds specifically to the incorporated unlabeled first type of nucleotides, and the second labeling reagent binds specifically to the incorporated unlabeled second type of nucleotides to provide labeled extended copy polynucleotides. In some such embodiments, the first functional moiety of the first type of unlabeled nucleotide is bound to the first labeling reagent by covalent bonding, optionally via a cleavable linker as described herein. In some other embodiments, the second functional moiety of the first type of unlabeled nucleotide is bound to the second labeling reagent by noncovalent interaction, optionally via the cleavable linker as described herein. In some such embodiments, the third type of unlabeled nucleotides comprises a third functional moiety, wherein the aqueous labeling mixture comprises a third labeling reagent, and the third labeling reagent comprises one or more third detectable labels and a third binding moiety that is capable of specific binding to the third functional moiety of the third type of unlabeled nucleotides. In some such embodiments, step (c′) is performed under conditions where the first labeling reagent binds specifically to the incorporated unlabeled first type of nucleotides, the second labeling reagent binds specifically to the incorporated unlabeled second type of nucleotides, and the third labeling reagent binds specifically to the incorporated unlabeled third type of nucleotides to provide labeled extended copy polynucleotides. In some other embodiments, the third type of unlabeled nucleotide comprises a mixture of the third type of unlabeled nucleotides comprising the first functional moiety and the third type of unlabeled nucleotides comprising the second functional moiety, and wherein both the first labeling reagent and the second labeling reagent are capable of specific binding to the third type of unlabeled nucleotides. In further embodiments, the fourth type of unlabeled nucleotides is not capable of specific binding with any of the first, second, or third labeling reagent. Uses of unlabeled nucleotides and affinity/labeling reagents in sequencing by synthesis have been disclosed in WO 2018/129214 and WO 2020/097607, as well as U.S. Publication No. 2023/0383342 A1 and U.S. Ser. No. 18/820,008, each of which is incorporated by reference in its entirety.

In some embodiments of the any of the sequencing methods described herein, each type of nucleotides has a 3′-O-glycoside blocking group as described herein. In some embodiments, the 3′-O-glycoside group is removed by contacting the solid support with an aqueous cleavage solution comprising a glycoside hydrolase or glycosidase that is capable of catalyzing the hydrolysis of the —O-glycosidic bond of the nucleotide. In some embodiments, the glycoside hydrolase or glycosidase is an arabinofuranosidase, a glucosidase, a mannosidase, a xylosidase, a galactosidase, an N-acetyl-glucosaminidase, or a glucuronidase. In further embodiments, the concentration of the glycoside hydrolase or glycosidase in the aqueous cleavage solution is at about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 μM or within a range defined by any two of the preceding values. In some embodiments, the concentration of the glycoside hydrolase or glycosidase in the aqueous cleavage solution is at least about 0.1 μM. In some embodiments, the concentration of the glycoside hydrolase or glycosidase in the aqueous cleavage solution is at least about 0.5 μM. In some embodiments, the concentration of the glycoside hydrolase or glycosidase in the aqueous cleavage solution is at least about 1 μM. In some embodiments, the concentration of the glycoside hydrolase or glycosidase in the aqueous cleavage solution is at least about 1.5 μM. In some embodiments, the concentration of the glycoside hydrolase or glycosidase in the aqueous cleavage solution is at least about 2 μM. In some embodiments, the molar ratio of the enzyme (e.g., a glycoside hydrolase or glycosidase) to the 3′ blocked dNTP as described herein is about 10:1, 5:1, 1:1, 1:5, 1:10, 1:20, 1:30, 1:40, 1:50, 1:60, 1:70, 1:80, 1:90, 1:100, 1:150, 1:200, 1:250, 1:300, 1:350, 1:400, 1:450, or 1:500, or a range defined by any two of the preceding values. In further embodiments, the molar ratio of the enzyme to the 3′ blocked dNTP is about 1:10 or 1:100.

In some embodiments, step (e) or (f′) comprises enzymatically removing the 3′ blocking group from the nucleotides incorporated into the extended copy polynucleotides. In some embodiments, step (e) or (f′) is conducted at a temperature of 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C., or a within a range defined by any two of the preceding values. In some embodiments, step (e) or (f′) is conducted at a temperature of at least about 35° C. In some embodiments, step (e) or (f′) is conducted at a temperature of at least about 37° C. In some embodiments, step (e) or (f′) is conducted at a temperature of at least about 60° C. In some embodiments, step (e) or (f′) is conducted at a temperature of at least about 65° C. In some embodiments, step (e) or (f′) is conducted at a temperature of at least about 70° C. In some embodiments, step (e) or (f′) is conducted at a temperature of at least about 80° C. In some embodiments, step (e) or (f′) is conducted at a pH of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0, in in a range defined by any two of the preceding values. In some embodiments, step (e) or (f′) is conducted at a pH between about 5.5 and about 6.5. In some embodiments, step (e) or (f′) is conducted at a pH between about 5.9 and about 6.1. In some embodiments, step (e) or (f′) is conducted at a pH of about 6.0.

In any embodiments of the sequencing methods described herein, the fourth type of nucleotide is unlabeled. In some further embodiments, the G nucleotide is unlabeled. In some further embodiments, G nucleotide has a structure selected from the group consisting of:

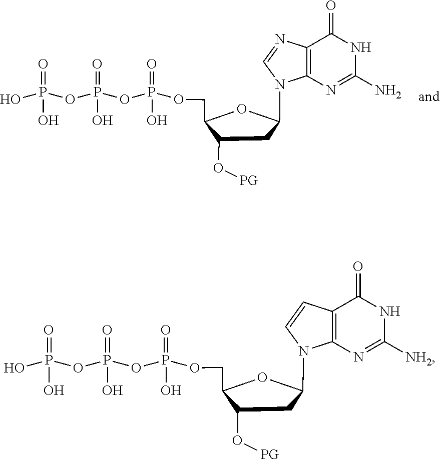

wherein PG stands for the glycosidyl blocking group as described herein.

In any embodiments of the sequencing methods described herein, the solid support comprises at least 500,000, 1,000,000, 2,000,000, 3,000,000, 4,000,000, or 5,000,000 spatially distinguishable sites/cm² that comprise multiple copies of target polynucleotides.

### Incorporation Mix

In some embodiments of the method described herein, step (b) or (b′), also referred to as the incorporation step, includes contacting a mixture containing one or more nucleotides (e.g., dATP, dCTP, dGTP, and dTTP or dUTP) with a copy polynucleotide/target polynucleotide complex in an incorporation solution comprising a polymerase and one or more buffering agents. In some such embodiments, the polymerase is a DNA polymerase, such as a mutant of 9° N polymerase (e.g., those disclosed in WO 2005/024010, U.S. Publication Nos. 2020/0131484 A1, 2020/0181587 A1, and 2024/0141427, each of which is incorporated by reference herein in its entirety. The amino acid sequences of Pol 812, Pol 1901, Pol 1558 or Pol 963 DNA polymerases are described, for example, in U.S. Patent Publication Nos. 2020/0131484 A1 and 2020/0181587 A1, both of which are incorporated by reference herein. The amino acid sequence of Pol A is disclosed as SEQ ID NO:5 of U.S. Publication No. 2024/0141427. In some embodiments, the one or more buffering agents comprise a primary amine, a secondary amine, a tertiary amine, a natural amino acid, or a non-natural amino acid, or combinations thereof. In further embodiments, the buffering agents comprise ethanolamine or glycine, or a combination thereof. In one embodiment, the buffer agent comprises or is glycine. In some embodiments, the use of glycine in the incorporation mix may improve the phasing value, as compared to standard buffering agent such as ethanolamine (EA) at the same condition. For example, the use of glycine provides a reduction or decrease in phasing value of at least 10%, 20%, 30%, 40%, 50%, 60%, 70%, 80%, 90%, 100%, 200%, 300%, 400%, or 500% as compared to as ethanolamine used under the same condition. In some instances, the use of glycine provides a % phasing value of less than about 0.15%, 0.1%, 0.09%, 0.08%, 0.07%, 0.06%, 0.05%, 0.04%, 0.03%, 0.02%, or 0.01% in a SBS sequencing run of at least 50 cycles. In further embodiments, the use of glycine provides a % phasing value of less than about 0.08% in Read 1 of a SBS sequencing run of at least 150 cycles. In further embodiments, the mutant of 9° N polymerase may be engineered for highly efficient incorporation of the nucleotide with the 3′-O-glycoside blocking group.

### Cleavage Mix

In some embodiments of the method described herein, step (e) of (f′), also referred to as the cleaving step, includes contacting the incorporated nucleotide and the copy polynucleotide strand with a cleavage solution comprising an enzyme described herein. In some embodiments, the cleavage solution comprises a catalyst or enzyme capable of cleaving the linker group in accordance with the present disclosure. In some embodiments, the cleavage solution comprises an enzyme capable of cleaving the linker described herein. In further embodiments, the linker is a self-immolative linker and the enzyme is capable of triggering the self-immolative linker. In some such embodiments, the 3′-OH blocking group and the detectable label are removed in a single step of reaction.

### Kits

One aspect of the present disclosure relates to a kit comprising one or more nucleotides according to the present disclosure.

In some embodiments, the kit further comprises a first enzyme, wherein the first enzyme is a polymerase, a terminal deoxynucleotidyl transferase, or a reverse transcriptase. In further embodiments, the first enzyme and the one or more nucleotides are in a first compartment of the kit. In some embodiments, the polymerase is a DNA polymerase as described herein. In some embodiments, the kit further comprises a second enzyme for removing the 3′-O-glycoside blocking group of the nucleotide, wherein the second enzyme is in a separate compartment from the first enzyme. In further embodiments, the second enzyme is a glycoside hydrolase or glycosidase that is capable of catalyzing the hydrolysis of the —O-glycosidic bond of the nucleotide. In further embodiments, the second enzyme is an arabinofuranosidase, a glucosidase, a mannosidase, a xylosidase, a galactosidase, an N-acetyl-glucosaminidase, or a glucuronidase. In yet further embodiments, the second enzyme is an L-arabinofuranosidase, a D-glucosidase, a D-mannosidase, a D-xylosidase, a D-galactosidase, an N-acetyl-D-glucosaminidase, or a D-glucuronidase. In yet further embodiments, the second enzyme is an a-L-arabinofuranosidase, a P-L-arabinofuranosidase, an a-D-glucosidase, a P-D-glucosidase, an a-D-mannosidase, a P-D-mannosidase, an a-D-xylosidase, a P-D-xylosidase, an a-D-galactosidase, a P-D-galactosidase, an a-N-acetyl-D-glucosaminidase, a P—N-acetyl-D-glucosaminidase, an a-D-glucuronidase, or a P-D-glucuronidase. In some embodiments, the kit may further include a chemical reagent or a third enzyme for removing the cleavable linker. In other embodiments, the second enzyme that may also remove the linker group (e.g., the cleavable linker is a self-immolative linker that can degrade under the stimuli of the second enzyme).

In some embodiments, the kit may contain four types of labeled nucleotides (A, C, G and T or U), where one or more of the four types of nucleotides is labeled. In such a kit, each of the four types of nucleotides can be labeled with a compound that is the same or different from the label on the other three nucleotides. Alternatively, a first type of the four types of nucleotides carries a first label, a second type of nucleotides carries a second label, a third type of nucleotide carries a third label, and a fourth nucleotide is unlabeled (dark). As another example, a first type of nucleotide carries a first label, a second type of nucleotide carries a second label, a third nucleotide is a mixture of the third type of nucleotide carrying the first label and the third type of nucleotide carrying the second label, and a fourth nucleotide is unlabeled (dark). Thus, one or more of the label nucleotides can have a distinct absorbance maximum and/or emission maximum such that the compound(s) is(are) distinguishable from other compounds. For example, each compound can have a distinct absorbance maximum and/or emission maximum such that each of the compounds is spectrally distinguishable from the other three compounds (or two compounds if the fourth nucleotide is unlabeled). It will be understood that parts of the absorbance spectrum and/or emission spectrum other than the maxima can differ and these differences can be exploited to distinguish the compounds. The kit may be such that two or more of the compounds have a distinct absorbance maximum.

The compounds, nucleotides, or kits that are set forth herein may be used to detect, measure, or identify a biological system (including, for example, processes or components thereof). Exemplary techniques that can employ the compounds, nucleotides or kits include sequencing, expression analysis, hybridization analysis, genetic analysis, RNA analysis, cellular assay (e.g., cell binding or cell function analysis), or protein assay (e.g., protein binding assay or protein activity assay). The use may be on an automated instrument for carrying out a particular technique, such as an automated sequencing instrument. The sequencing instrument may contain two light sources operating at different wavelengths.

In a particular embodiment, the labeled nucleotide(s) described herein may be supplied in combination with unlabeled or native nucleotides, or any combination thereof. Combinations of nucleotides may be provided as separate individual components (e.g., one nucleotide type per vessel or tube) or as nucleotide mixtures (e.g., two or more nucleotides mixed in the same vessel or tube).

Where kits comprise a plurality, particularly two, or three, or more particularly four, nucleotides, the different nucleotides may be labeled with different dye compounds, or one may be dark, with no dye compounds. Where the different nucleotides are labeled with different dye compounds, it is a feature of the kits that the dye compounds are spectrally distinguishable fluorescent dyes. As used herein, the term “spectrally distinguishable fluorescent dyes” refers to fluorescent dyes that emit fluorescent energy at wavelengths that can be distinguished by fluorescent detection equipment (for example, a commercial capillary-based DNA sequencing platform) when two or more such dyes are present in one sample. When two nucleotides labeled with fluorescent dye compounds are supplied in kit form, it is a feature of some embodiments that the spectrally distinguishable fluorescent dyes can be excited at the same wavelength, such as, for example by the same light source. When four nucleotides labeled with fluorescent dye compounds are supplied in kit form, it is a feature of some embodiments that two of the spectrally distinguishable fluorescent dyes can both be excited at one wavelength and the other two spectrally distinguishable dyes can both be excited at another wavelength. Particular excitation wavelengths for the dyes are between 450-460 nm, 490-500 nm, or 520 nm or above (e.g., 532 nm).

### Post-Incorporation Labeling Kits

In other embodiments, one or more types of nucleotides A, G, C, and T or U are unlabeled, and wherein the first type of unlabeled nucleotides comprises a first functional moiety, and the kit further comprises a first labeling reagent, wherein the first labeling reagent comprises one or more first detectable labels and a first binding moiety that is capable of specific binding to the first functional moiety of the first type of unlabeled nucleotide. In some such embodiments, the first functional moiety of the first type of unlabeled nucleotide is bound to the first labeling reagent by either covalent bonding or noncovalent interaction via a cleavable linker described herein. In some embodiments, two or more types of nucleotides A, G, C, and T or U are unlabeled. In some further embodiments, each of the four types of nucleotides is unlabeled, and wherein the second type of unlabeled nucleotides comprises a second functional moiety, and the kit further comprises a second labeling reagent, wherein the second labeling reagent comprises one or more second detectable labels and a second binding moiety that is capable of specific binding to the second functional moiety of the second type of unlabeled nucleotide. In some such embodiments, the second functional moiety of the second type of unlabeled nucleotide is bound to the second labeling reagent by either covalent bonding or noncovalent interaction via a cleavable linker described herein. In some further embodiments, the third type of unlabeled nucleotides comprises a third functional moiety, and the kit further comprises a third labeling reagent, wherein the third labeling reagent comprises one or more third detectable labels and a third binding moiety that is capable of specific binding to the third functional moiety of the third type of unlabeled nucleotide. In some such embodiments, the third functional moiety of the third type of unlabeled nucleotide is bound to the third labeling reagent by either covalent bonding or noncovalent interaction via a cleavable linker described herein. In other embodiments, the third type of unlabeled nucleotides comprises a mixture of the third type of unlabeled nucleotides comprising the first functional moiety and the third type of unlabeled nucleotides comprising the second functional moiety, and wherein both the first labeling reagent and the second labeling reagent are capable of specific binding to the third type of unlabeled nucleotides. In some further embodiments, the fourth type of unlabeled nucleotides is not capable of specific binding with any of the first, second, or third labeling reagent. Post-incorporation labeling kits and methods have been described in U.S. Publication No. 2023/0383342 A1 and U.S. Ser. No. 18/820,008, both of which are incorporated by reference in its entirety. Non-limiting examples of noncovalent interaction between a functional moiety of the nucleotide and a binding moiety of the labeling reagent include but are not limited to avidin (e.g., streptavidin or neutravidin) and biotin; dinitrophenyl (DNP) moiety and anti-DNP antibody; digoxigenin (DIG) and anti-DIG antibody; P—N-acetyl glucosamine (0-GlcNAc) and WGA (lectin); alkyl guanine moiety and SNAP-Tag®, alkyl chloride moiety and HaloTag®; 3-nitrotyrosine and anti-nitrotyrosine antibody; nickel or cobalt complex such as Ni-nitrilotriacetic acid (NTA) and His-Tag; zinc complex and oligo-aspartate protein. Non-limiting examples of covalent interaction between a functional moiety and a labeling reagent include but are not limited to a biorthogonal reaction selected from the group consisting of a [3+2]dipolar cycloaddition, a Diels-Alder cycloaddition, a [4+1]cycloaddition, a phosphine ligation, or condensation with 2-acylphenyl boronic acid. For example, one of the functional moiety and the binding moiety comprises or is norbornene, transcyclooctene (TCO), dibenzocyclooctyne (DBCO), or bicyclo[6.1.0]nonyne (BCN), and the other one of the functional moiety and the binding moiety comprises or is azido. In some other embodiments, one of the functional moiety and the binding moiety comprises or is TCO, and the other one of the functional moiety and the binding moiety comprises or is an optionally substituted 1,2,4,5-tetrazine moiety.

### Embodiments and Alternatives of Sequencing-by-Synthesis

Alternatively, the sequencing methods described herein may also be carried out using unlabeled nucleotides and affinity reagents containing a fluorescent dye described herein. For example, one, two, three or each of the four different types of nucleotides (e.g., dATP, dCTP, dGTP and dTTP or dUTP) in the incorporation mixture of step (1) may be unlabeled. Each of the four types of nucleotides (e.g., dNTPs) has a 3′ blocking group to ensure that only a single base can be added by a polymerase to the 3′ end of the primer polynucleotide. After incorporation of an unlabeled nucleotide, the remaining unincorporated nucleotides are washed away. An affinity reagent is then introduced that specifically recognizes and binds to the incorporated dNTP to provide a labeled extension product comprising the incorporated dNTP. Uses of unlabeled nucleotides and affinity reagents in sequencing by synthesis have been disclosed in WO 2018/129214 and WO 2020/097607. A modified sequencing method of the present disclosure using unlabeled nucleotides may include the following steps:
(1) contacting a solid support with sequencing primers under hybridization conditions, wherein the solid support comprises a plurality of target polynucleotides immobilized thereon; and the sequencing primers are complementary to at least a portion of the target polynucleotides;
(2) contacting the solid support with an aqueous solution comprising DNA polymerase and one or more of four different types of unlabeled nucleotides (A, G, C and T or U) under conditions suitable for DNA polymerase-mediated primer extension, wherein each of the nucleotides comprises a 3′ blocking group and at least one type of nucleotide comprising 3′-O-glycoside blocking group as described herein;
(3) contacting the extended copy polynucleotides with a set of affinity reagents under conditions wherein one affinity reagent binds specifically to the incorporated unlabeled nucleotides to provide labeled extended copy polynucleotides;
(4) imaging the solid support to determine the identity of the incorporated nucleotides (e.g., by performing one or more fluorescent measurements of the extended copy polynucleotides); and
(5) contacting the solid support with an aqueous deblocking solution comprising a palladium catalyst and tris(hydroxyalkyl)phosphine under conditions suitable to chemically remove 3′ allyl blocking groups from incorporated nucleotides to expose a 3′-OH group for further nucleotide incorporation on the solid support; and
repeating steps (2)-(5) to determine target polynucleotide sequences.

In some embodiments of the modified sequencing method described herein, the method further comprises removing the affinity reagents from the incorporated nucleotides. In still further embodiments, the 3′ blocking group and the affinity reagent are removed in the same reaction. In some embodiments, the method further comprises a step (6) washing the solid support with a third aqueous wash solution. In further embodiments, steps (2) through (6) are repeated at least 50, 100, 150, 200, 250 or 300 cycles to determine the target polynucleotide sequences. In some embodiments, the set of affinity reagents may comprise a first affinity reagent that binds specifically to the first type of nucleotide, a second affinity reagent that binds specifically to the second type of nucleotide, and a third affinity reagent that binds specifically to the third type of nucleotide. In some further embodiments, each of the first, second and the third affinity reagents comprises a detectable labeled that is spectrally distinguishable. In some embodiments, the affinity reagents may include protein tags, antibodies (including but not limited to binding fragments of antibodies, single chain antibodies, bispecific antibodies, and the like), aptamers, knottins, affimers, or any other known agent that binds an incorporated nucleotide with a suitable specificity and affinity. In one embodiment, at least one affinity reagent is an antibody or a protein tag. In another embodiment, at least one of the first type, the second type, and the third type of affinity reagents is an antibody or a protein tag comprising one or more detectable labels (e.g., multiple copies of the same detectable label).

Some embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina, Inc.), and is also described in WO 91/06678 and WO 07/123,744, each of which is incorporated herein by reference. The availability of fluorescently-labeled terminators in which both the termination can be reversed, and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially, and an image of the array can be obtained between each addition step. In such embodiments each image will show nucleic acid features that have incorporated nucleotides of a particular type. Different features will be present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed, and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

Some embodiments can utilize detection of four different nucleotides using fewer than four different labels. For example, SBS can be performed utilizing methods and systems described in the incorporated materials of U.S. Pub. No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Pub. No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”, Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some other embodiments of sequencing methods involve the use the 3′ blocked nucleotide described herein in nanoball sequencing technique, such as those described in U.S. Pat. No. 9,222,132, the disclosure of which is incorporated by reference. Through the process of rolling circle amplification (RCA), a large number of discrete DNA nanoballs may be generated. The nanoball mixture is then distributed onto a patterned slide surface containing features that allow a single nanoball to associate with each location. In DNA nanoball generation, DNA is fragmented and ligated to the first of four adapter sequences. The template is amplified, circularized and cleaved with a type II endonuclease. A second set of adapters is added, followed by amplification, circularization and cleavage. This process is repeated for the remaining two adapters. The final product is a circular template with four adapters, each separated by a template sequence. Library molecules undergo a rolling circle amplification step, generating a large mass of concatemers called DNA nanoballs, which are then deposited on a flow cell. Goodwin et al., “Coming of age: ten years of next-generation sequencing technologies,” Nat Rev Genet. 2016; 17(6):333-51.

Some embodiments can utilize methods involving the real-time monitoring of DNA polymerase activity. Nucleotide incorporations can be detected through fluorescence resonance energy transfer (FRET) interactions between a fluorophore-bearing polymerase and 7-phosphate-labeled nucleotides as described, for example, in U.S. Pat. Nos. 7,329,492 and 7,211,414, both of which are incorporated herein by reference, or nucleotide incorporations can be detected with zero-mode waveguides as described, for example, in U.S. Pat. No. 7,315,019, which is incorporated herein by reference, and using fluorescent nucleotide analogs and engineered polymerases as described, for example, in U.S. Pat. No. 7,405,281 and U.S. Pub. No. 2008/0108082, both of which are incorporated herein by reference. The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in U.S. Pub. Nos. 2009/0026082; 2009/0127589; 2010/0137143; and 2010/0282617, all of which are incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in U.S. Pub. Nos. 2010/0111768 and 2012/0270305, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Pub. No. 2012/0270305, which is incorporated herein by reference.

Arrays in which polynucleotides have been directly attached to silica-based supports are those for example disclosed in WO 00/06770 (incorporated herein by reference), wherein polynucleotides are immobilized on a glass support by reaction between a pendant epoxide group on the glass with an internal amino group on the polynucleotide. In addition, polynucleotides can be attached to a solid support by reaction of a sulfur-based nucleophile with the solid support, for example, as described in WO 2005/047301 (incorporated herein by reference). A still further example of solid-supported template polynucleotides is where the template polynucleotides are attached to hydrogel supported upon silica-based or other solid supports, for example, as described in WO 00/31148, WO 01/01143, WO 02/12566, WO 03/014392, U.S. Pat. No. 6,465,178 and WO 00/53812, each of which is incorporated herein by reference.

A particular surface to which template polynucleotides may be immobilized is a polyacrylamide hydrogel. Polyacrylamide hydrogels are described in the references cited above and in WO 2005/065814, which is incorporated herein by reference. Specific hydrogels that may be used include those described in WO 2005/065814 and U.S. Pub. No. 2014/0079923. In one embodiment, the hydrogel is PAZAM (poly(N-(5-azidoacetamidylpentyl) acrylamide-co-acrylamide)).

DNA template molecules can be attached to beads or microparticles, for example, as described in U.S. Pat. No. 6,172,218 (which is incorporated herein by reference). Attachment to beads or microparticles can be useful for sequencing applications. Bead libraries can be prepared where each bead contains different DNA sequences. Exemplary libraries and methods for their creation are described in Nature, 437, 376-380 (2005); Science, 309, 5741, 1728-1732 (2005), each of which is incorporated herein by reference. Sequencing of arrays of such beads using nucleotides set forth herein is within the scope of the disclosure.

Templates that are to be sequenced may form part of an “array” on a solid support, in which case the array may take any convenient form. Thus, the method of the disclosure is applicable to all types of high-density arrays, including single-molecule arrays, clustered arrays, and bead arrays. Labeled nucleotides of the present disclosure may be used for sequencing templates on essentially any type of array, including but not limited to those formed by immobilization of nucleic acid molecules on a solid support.

However, labeled nucleotides of the disclosure are particularly advantageous in the context of sequencing of clustered arrays. In clustered arrays, distinct regions on the array (often referred to as sites, or features) comprise multiple polynucleotide template molecules. Generally, the multiple polynucleotide molecules are not individually resolvable by optical means and are instead detected as an ensemble. Depending on how the array is formed, each site on the array may comprise multiple copies of one individual polynucleotide molecule (e.g., the site is homogenous for a particular single- or double-stranded nucleic acid species) or even multiple copies of a small number of different polynucleotide molecules (e.g., multiple copies of two different nucleic acid species). Clustered arrays of nucleic acid molecules may be produced using techniques generally known in the art. By way of example, WO 98/44151 and WO 00/18957, each of which is incorporated herein, describe methods of amplification of nucleic acids wherein both the template and amplification products remain immobilized on a solid support in order to form arrays comprised of clusters or “colonies” of immobilized nucleic acid molecules. The nucleic acid molecules present on the clustered arrays prepared according to these methods are suitable templates for sequencing using the nucleotides labeled with dye compounds of the disclosure.

The labeled nucleotides of the present disclosure are also useful in sequencing of templates on single molecule arrays. The term “single molecule array” or “SMA” as used herein refers to a population of polynucleotide molecules, distributed (or arrayed) over a solid support, wherein the spacing of any individual polynucleotide from all others of the population is such that it is possible to individually resolve the individual polynucleotide molecules. The target nucleic acid molecules immobilized onto the surface of the solid support can thus be capable of being resolved by optical means in some embodiments. This means that one or more distinct signals, each representing one polynucleotide, will occur within the resolvable area of the particular imaging device used.

Single molecule detection may be achieved wherein the spacing between adjacent polynucleotide molecules on an array is at least 100 nm, more particularly at least 250 nm, still more particularly at least 300 nm, even more particularly at least 350 nm. Thus, each molecule is individually resolvable and detectable as a single molecule fluorescent point, and fluorescence from said single molecule fluorescent point also exhibits single step photobleaching.

The terms “individually resolved” and “individual resolution” are used herein to specify that, when visualized, it is possible to distinguish one molecule on the array from its neighboring molecules. Separation between individual molecules on the array will be determined, in part, by the particular technique used to resolve the individual molecules. The general features of single molecule arrays will be understood by reference to published applications WO 00/06770 and WO 01/57248, each of which is incorporated herein by reference. Although one use of the nucleotides of the disclosure is in sequencing-by-synthesis reactions, the utility of the nucleotides is not limited to such methods. In fact, the nucleotides may be used advantageously in any sequencing methodology which requires detection of fluorescent labels attached to nucleotides incorporated into a polynucleotide.

In particular, the labeled nucleotides of the disclosure may be used in automated fluorescent sequencing protocols, particularly fluorescent dye-terminator cycle sequencing based on the chain termination sequencing method of Sanger and co-workers. Such methods generally use enzymes and cycle sequencing to incorporate fluorescently labeled dideoxynucleotides in a primer extension sequencing reaction. So-called Sanger sequencing methods, and related protocols (Sanger-type), utilize randomized chain termination with labeled dideoxynucleotides.

Thus, the present disclosure also encompasses labeled nucleotides which are dideoxynucleotides lacking hydroxyl groups at both of the 3′ and 2′ positions, such dideoxynucleotides being suitable for use in Sanger type sequencing methods and the like.

Labeled nucleotides of the present disclosure incorporating 3′ blocking groups, it will be recognized, may also be of utility in Sanger methods and related protocols since the same effect achieved by using dideoxy nucleotides may be achieved by using nucleotides having 3′-OH blocking groups: both prevent incorporation of subsequent nucleotides. Where nucleotides according to the present disclosure, and having a 3′ blocking group are to be used in Sanger-type sequencing methods it will be appreciated that the dye compounds or detectable labels attached to the nucleotides need not be connected via cleavable linkers, since in each instance where a labeled nucleotide of the disclosure is incorporated; no nucleotides need to be subsequently incorporated and thus the label need not be removed from the nucleotide.

### EXAMPLES

Additional embodiments are disclosed in further detail in the following examples, which are not in any way intended to limit the scope of the claims.

### Example 1. Syntheses of 3′-ABF Nucleoside, Nucleotide and Fully Functionalized Nucleotide

### Synthesis of ABF-STol

The synthesis of 1,2,3,5-tetraacetyl-L-arabinofuranose and arabinofuranose-STol (ABF-Stol) is described in Scheme 1.

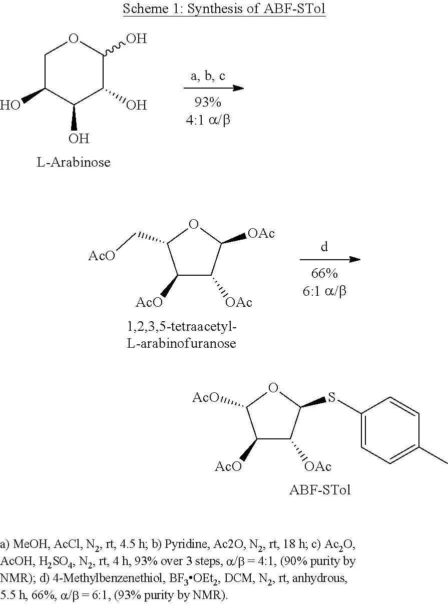 [SMILES: OC1OC[C@H](O)[C@H](O)[C@H]1O ; CC(=O)OC[C@@H]1O[C@@H](OC(C)=O)[C@H](OC(C)=O)[C@H]1OC(C)=O ; CC(=O)O[C@@H]1O[C@@H](Sc2ccc(C)cc2)[C@H](OC(C)=O)[C@H]1OC(C)=O]

### Synthesis of 1,2,3,5-tetraacetyl-L-arabinofuranose

L-Arabinofuranose (“L-ABF,” 5 g, 33.3 mmol) was suspended in anhydrous methanol (100 mL) under nitrogen and cooled to 0° C. Acetyl chloride (3 mL, 42 mmol) was added slowly dropwise, and the solution warmed to room temperature and stirred for 4.5 hours. The clear solution was cooled to 0° C. and quenched with pyridine (40 mL). Volatiles were removed in-vacuo and residue was co-evaporated twice with dichloromethane (40 mL) to yield crude 1-methoxy-L-arabinofuranose. The crude 1-methoxy-L-arabinofuranose was brought under nitrogen and dissolved in anhydrous pyridine (40 mL). The solution was cooled to 0° C., and acetic anhydride was added. The solution was stirred at room temperature overnight. Volatiles were removed under reduced pressure and residue was dissolved in dichloromethane (300 mL). The organic was washed with water (300 mL), 1 M solution of hydrochloric acid in water (300 mL), and saturated aqueous sodium bicarbonate solution (300 mL). Aqueous phases were each extracted with 50 mL dichloromethane and combined organic phases were washed with brine (150 mL) and dried over magnesium sulphate. The solid was filtered off and filtrate was evaporated under reduced pressure to yield crude 1-methoxy-2,3,5-triacetyl-L-arabinofuranose as a syrup that was used without further purification. The syrup was brought under nitrogen, dissolved in acetic acid (50 mL), acetic anhydride (12.5 mL), and cooled to 0° C. Neat sulfuric acid (5 mL) was added slowly dropwise and the solution was warmed to room temperature and stirred for 4 hours. The reaction was quenched with ice water (100 mL), stirred for 30 minutes, and aqueous extracted twice with dichloromethane (200 mL then 100 mL). The solution was combined organic washed with water (100 mL), saturated aqueous sodium bicarbonate (100 mL), brine (50 mL), and then dried over magnesium sulphate. The solvent was removed in-vacuo. The crude compound was purified by flash column chromatography on silica gel, eluting with hexanes/ethyl acetate to yield a clear syrup (93% yield: 9.85 g, 30.9 mmol; LC-MS (ESI): (positive ion) m/z=341.21 [M+Na⁺]).

### Synthesis of ABF-STol, 1-α-S-(4-Methylbenzyl)-2,3,5-O-triacetyl-L-arabinofuranose

1,2,3,5-tetraacetyl-L-arabinofuranose (6.97 g, 21.9 mmol) and 4-methylbenzenethiol (3.27 g, 26.3 mmol) were dried together in a flask in a desiccator over P₂O₅ at high vacuum for 24 hours. Freshly activated molecular sieves were added and the flask was sealed and purged 5 times with nitrogen. The compounds were dissolved in anhydrous dichloromethane (110 mL, dried over molecular sieves), cooled to 0° C., and stirred for 15 minutes. Boron trifluoride diethyl etherate (13.8 mL, 109.5 mmol) was added slowly via a desiccated syringe. The solution was warmed to room temperature and stirred for 5 hours. Amber solution was cooled to 0° C., quenched with triethylamine (30 mL), and stirred at room temperature for 5 minutes. The volatiles were removed under reduced pressure, and the dark coloured residue was dissolved in ethyl acetate (200 mL) and organic washed twice with water (2×200 mL). The aqueous phase was back-extracted with ethyl acetate (100 mL). The combined organic was washed with brine (100 mL), and dried over magnesium sulphate. The solid was filtered off and the solvent was removed in-vacuo. The crude compound was purified by flash column chromatography on silica gel eluting with hexanes/ethyl acetate to yield a clear amber syrup (66% yield (5.58 g, 14.59 mmol); LC-MS (ESI): (negative ion) m/z=381.26 [M−H⁺]⁻).

### Synthesis of 3′-ABF-Deoxythymidine

The synthesis of 3′-ABF-Thymidine is described in Scheme 2.

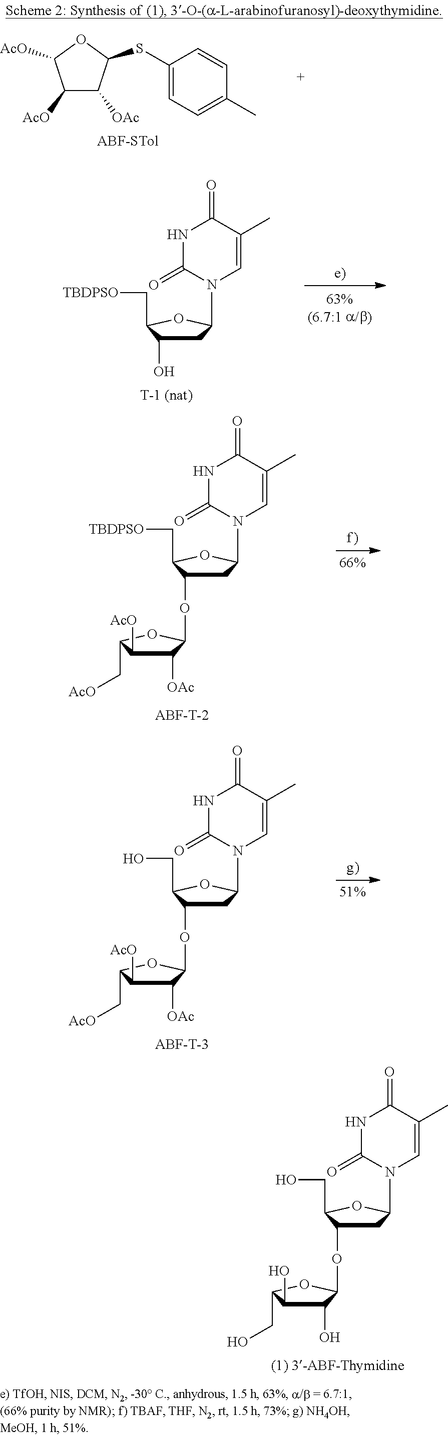

### Synthesis of ABF-T-2, 3′-O-(2″,3″,5″-O-triacetyl-α-L-arabinofuranosyl)-5′-O-tertbutyldiphenylsilyl-deoxythymidine

ABF-STol (100 mg, 0.261 mmol), known intermediate T-1 (nat) (188 mg, 0.391 mmol), and separately N-iodosuccinimide (65 mg, 0.288 mmol) were dried in a desiccator over P₂O₅ at high vacuum for 24 hours. N-iodosuccinimide was added to the donor and acceptor, and the flask was sealed and purged 5 times with nitrogen. The compounds were dissolved in anhydrous dichloromethane (2 mL, dried over molecular sieves), cooled to −30° C. and stirred for 15 minutes. Triflic acid (23 μL, 0.261 mmol) was added slowly via a desiccated syringe and the dark solution was stirred at −30° C. for 1.5 hours. The reaction was quenched slowly with triethylamine (200 μL), warmed to room temperature, and stirred for 5 minutes. The reaction was diluted with dichloromethane (10 mL) and washed with saturated aqueous sodium bicarbonate solution (10 mL). The aqueous phase was extracted twice with dichloromethane (2×5 mL) and the combined organic was dried over magnesium sulphate, filtered, and concentrated in-vacuo to give a dark crude compound. This was purified by flash column chromatography on silica gel eluting with hexanes/ethyl acetate to yield the product as a 2:1 mixture of product and T-1 (nat) starting material (63% yield: 162 mg, 66% purity, 0.166 mmol; LC-MS (ESI): (negative ion) m/z=737.63 [M−H⁺]⁻, (positive ion) m/z=739.56 [M+H⁺]).

### Synthesis of ABF-T-3, 3′-O-(2″,3″,5″-O-triacetyl-α-L-arabinofuranosyl)-deoxythymidine

Impure ABF-T-3 (0.165 mmol) was dissolved as in anhydrous tetrahydrofuran (3 mL) under nitrogen, a 1M solution of tetrabutylammonium fluoride in tetrahydrofuran (298 μL, 0.298 mmol) was added and the reaction was stirred at room temperature for 1.5 hours. The reaction was diluted with ethyl acetate (10 mL) and organic washed twice with water (2×10 mL). The aqueous phase was back-extracted with ethyl acetate (15 mL). The combined organic was washed with brine, dried over magnesium sulphate, filtered, and the solvents were removed under reduced pressure to give an amber crude compound. The amber crude compound was purified by flash column chromatography on silica gel eluting with dichloromethane/methanol to yield the product as a clear oil (66% yield: 57 mg, 0.109 mmol; LC-MS (ESI): (negative ion) m/z=499.42 [M−H⁺]⁻, (positive ion) m/z=501.45 [M+H⁺]).

### Synthesis of (1) 3′-ABF-Deoxythymidine, 3′-O-(α-L-arabinofuranosyl)-deoxythymidine

ABF-T-4 (50 mg, 0.1 mmol) was dissolved in methanol (1 mL), ammonium hydroxide solution (35%, 2 mL) was added, and the mixture was stirred at room temperature for 1.5 hours until all starting material was consumed. Reaction was diluted with water (5 mL) and all volatiles removed under vacuum. Product was purified by reverse phase chromatography on C18 modified silica, and further purified by preparative HPLC, eluting with 0.1 M TEAB solution and acetonitrile to yield pure 3′-ABF-Thymidine (51% yield: 50.6 μmol, determined by UV-Vis spectrometry, λₘₐₓ=266 nm, ε=9600 M-1 cm-1; LC-MS (ESI): (negative ion) m/z=373.34 [M−H⁺]⁻, (positive ion) m/z=375.30 [M+H⁺]).

### Synthesis of 3′-ABF-dTTP

The synthesis of 3′-ABF-dTTP is described in Scheme 3.

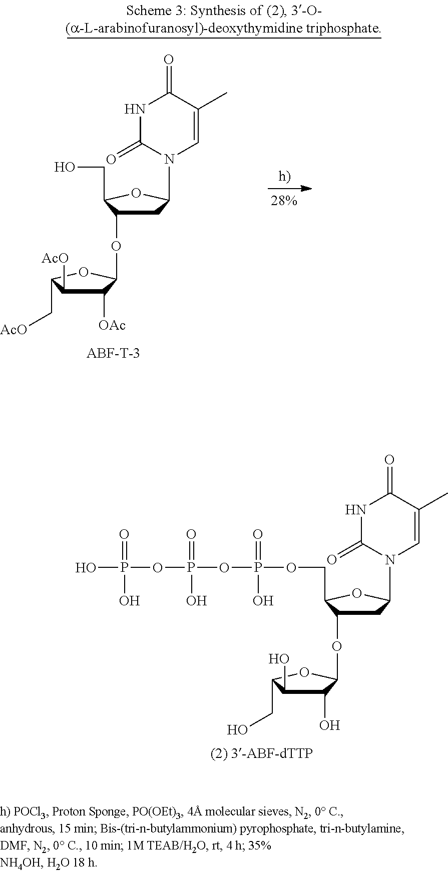

### Synthesis of (2) 3′-ABF-dTTP, 3′-O-(α-L-arabinofuranosyl)-deoxythymidine-5′-triphosphate

ABF-T-3 (21.1 mg, 42.1 μmol), proton sponge (16 mg, 75.8 μmol), and a magnetic stirrer were dried together in a desiccator over P₂O₅ overnight. Freshly activated 4 Å molecular sieves were added and the components were brought under nitrogen. Anhydrous triethyl phosphate (250 μL) was added and the mixture was cooled to 0° C. Freshly distilled POCl₃ (4.7 μL, 50.5 μmol) was added dropwise and the reaction was stirred at 0° C. for 15 minutes. A solution of bis-(tri-n-butylammonium) pyrophosphate (115 mg, 210 μmol) in minimal anhydrous N,N-dimethylformamide was added, followed immediately by anhydrous tri-n-butylamine (45 μL, 189 μmol). The mixture was vigorously stirred for 10 minutes at 0° C. before being quenched by addition of an aqueous solution of 2 μM TEAB (715 μL). The solution was transferred to a separate flask, the reaction vessel was rinsed with water (715 μL), and the washings added into the 2 M TEAB solution. The combined mixture was then stirred at room temperature for 4 hours, after which the solvent was evaporated under reduced pressure. The residue was dissolved in water (750 μL) and ammonium hydroxide solution (35%, 1.5 mL) was added and the solution was stirred at room temperature overnight. The reaction was concentrated under vacuum and product separated by flash chromatography on DEAE Sephadex. Fractions containing the triphosphate were dried under vacuum and the crude was further purified by preparative HPLC to give pure 3′-ABF-TTP (28% yield: 11.9 μmol, determined by UV-Vis spectrometry, λₘₐₓ=266 nm, ε=9600 M⁻¹ cm⁻¹; LC-MS (ESI): (negative ion) m/z=613.22 [M−H⁺]⁻).

### Synthesis of ffT-PA-ABF-LN3-AF55POPOso

The synthesis of ffT-PA-ABF-LN3-AF55POPOso is described in Scheme 4.

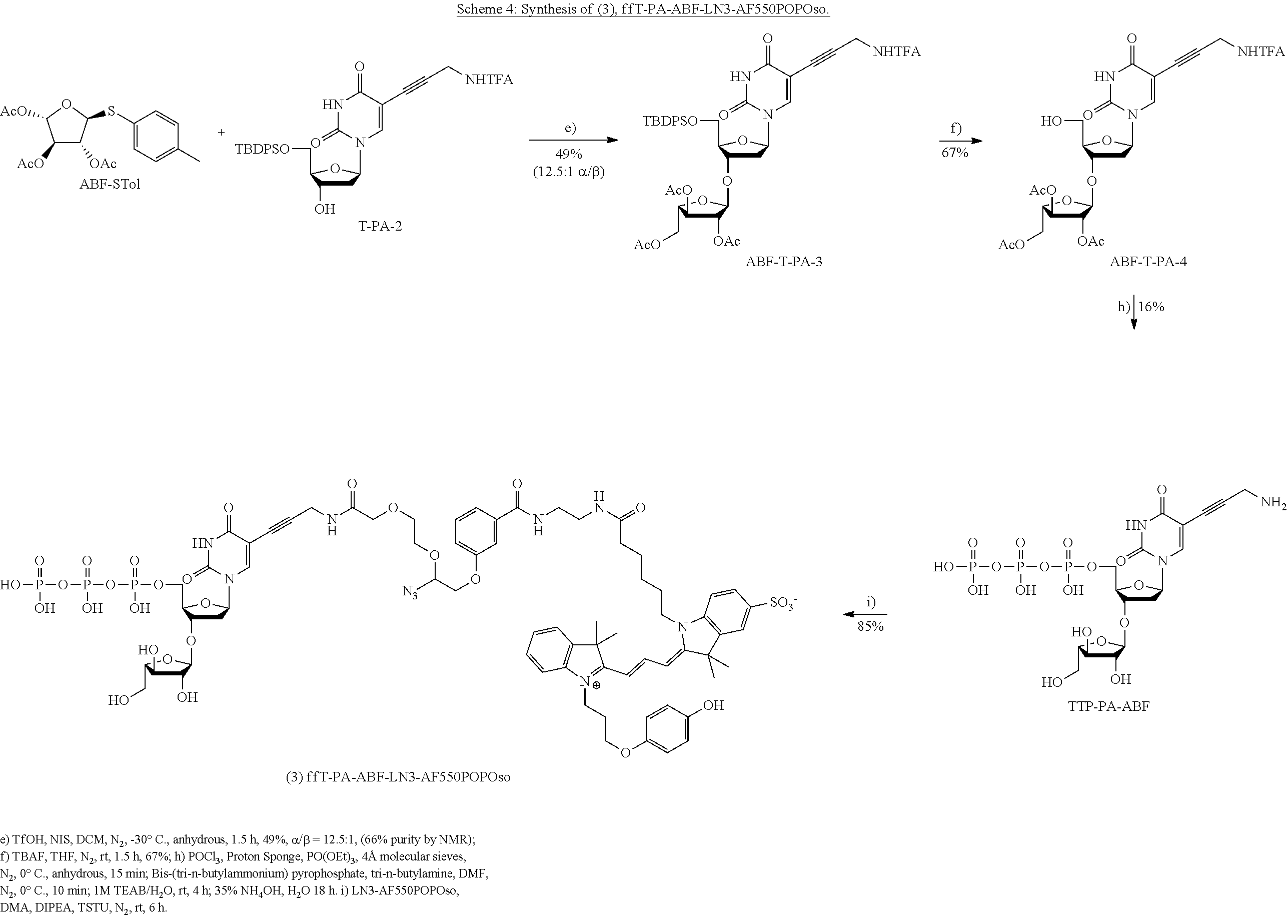

### Synthesis of ABF-T-PA-3, 3′-O-(2″,3″,5″-O-triacetyl-α-L-arabinofuranosyl)-5′-O-tertbutyldiphenylsilyl-5-(N-trifluoroacetamido-propargyl)-deoxythymidine

ABF-T-PA-3 was synthesised from known intermediate T-PA-2 (500 mg, 0.893 mmol) via the procedure outlined in the synthesis of ABF-T-2 (Scheme 2). ABF-T-PA-3 was collected as an off-white solid containing a 2:1 mixture of product and T-PA-2 starting material (49% yield: 513 mg, 66% purity, 0.436 mmol; LC-MS (ESI): (negative ion) m/z=872.67 [M−H⁺]⁻, (positive ion) m/z=874.62 [M+H⁺]).

### Synthesis of ABF-T-PA-4, 3′-O-(2″,3″,5″-O-triacetyl-α-L-arabinofuranosyl)-5-(N-trifluoroacetamido-propargyl)-deoxythymidine

ABF-T-PA-4 was synthesised from impure ABF-T-PA-3 (512 mg, 0.435 mmol) via the procedure outlined in the synthesis of ABF-T-3 (Scheme 2). Product obtained as a clear oil (67% yield: 231 mg, 0.293 mmol; LC-MS (ESI): (negative ion) m/z=634.32 [M−H⁺]⁻, (positive ion) m/z=636.32 [M+H⁺]).

### Synthesis of TTP-PA-ABF, 3′-O-(α-L-arabinofuranosyl)-5-(propargylamino)-deoxythymidine-5′-triphosphate

TP-PA-ABF was synthesised from ABF-T-PA-4 (230 mg, 0.293 mmol) Via the procedure outlined in the synthesis of (2) 3′-ABF-TTP (Scheme 3). Triphosphate obtained as a white solid (16% yield: 46.6 μmol, determined by UV-Vis spectrometry, λₘₐₓ=266 nm, F=9790 M⁻¹ cm⁻¹; LC-MS (ESI): (negative ion) m/z=652.16 [M−H⁺]⁻).

### Synthesis of ffT-PA-ABF-LN3-AF550POPOso

Known intermediate LN3-AF550POPOso (15 μmol) was dried under high vacuum for 1 h, brought under nitrogen and dissolved in anhydrous N,N-dimethylacetamide (3 mL). N,N-diisopropylethylamine (26 μL, 150 μmol) was added, followed by a 0.1 μM solution of N,N,N′,N′-Tetramethyl-O—(N-succinimidyl)uronium tetrafluoroborate in N,N-dimethylacetamide (16 μmol, 160 L). The reaction mixture was stirred at room temperature for 25 minutes, before being transferred via syringe to a sealed flask containing TTP-PA-ABF (6.5 μmol), suspended in minimal aqueous 0.1 M TEAB (50 μL), under nitrogen. Additional N,N-dimethylacetamide washes (2×0.5 mL) were used to ensure complete transfer of activated LN3-AF550POPOso, and the combined mixture was stirred at room temperature for a further 6.5 hours. Reaction was quenched with an aqueous solution of 0.1M TEAB (10 mL), stirred for 30 minutes and volatiles removed in-vacuo. Crude mixture was purified by flash column chromatography on DEAE Sephadex, and further purified by preparative HPLC to give pure ffT-PA-ABF-LN3-AF550POPOso as a dark pink solid (85% yield; 5.55 μmol, determined by UV-Vis spectrometry, λₘₐₓ=554 nm, ε=125000M⁻¹ cm¹; LC-MS (ESI): (negativeion) m/z=1657.81 [M−H⁺]⁻).

### Synthesis of BzABF-STol

The synthesis of BzABF-STol is described in Scheme 5.

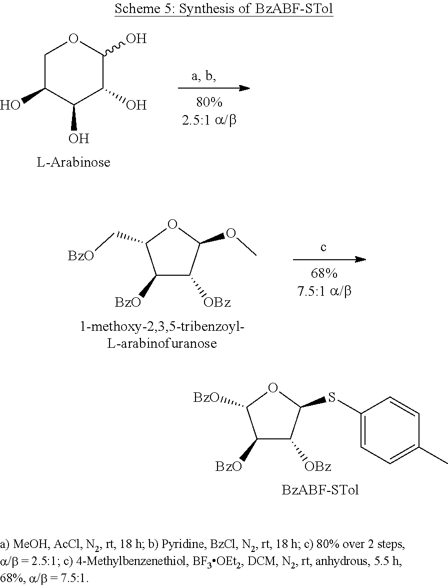

### Synthesis of 1-methoxy-2,3,5-tribenzoyl-L-arabinofuranose

L-Arabinofuranose (4.68 g, 31 mmol) was suspended in anhydrous methanol (50 mL) under nitrogen, acetyl chloride (0.7 mL, 10 mmol) was added slowly, dropwise, whilst vigorously stirring. The solution was stirred at room temperature overnight, after which it was quenched with pyridine (15 mL). The solvents were evaporated, co-evaporated with pyridine (5 mL), and the resulting crude compound was used without further purification. The crude compound was dissolved in anhydrous pyridine (50 mL) under nitrogen, cooled to 0° C. and benzoyl chloride (15 mL, 129 mmol) was added slowly, forming a precipitate. The suspension was warmed to room temperature and stirred overnight, before being quenched with water (1.5 mL). The mixture was diluted with dichloromethane (50 mL) and organic washed with water (50 mL), 1 M hydrochloric acid (3×50 mL until aqueous layer was strongly acidic), saturated aqueous sodium bicarbonate (50 mL), and brine (50 mL). The organic layer was separated, dried over magnesium sulfate, and evaporated to a crude product that was purified by flash column chromatography on silica gel, eluting with hexanes/ethyl acetate to yield a clear, viscous syrup (80% yield; 11.75 g, 24.6 mmol). ¹H NMR (400 MHz, CDCl₃) δ 8.04-7.90 (m, 6H, Ar—H), 7.56-7.47 (m, 2H, Ar—H), 7.47-7.27 (m, 6H, Ar—H), 7.26-7.20 (m, 1H, Ar—H), 5.51 (ddd, J=5.2, 1.4, 0.7 Hz, 1H, 3-H), 5.44 (d, J=1.4 Hz, 1H, 2-H), 5.11 (s, 1H, 1-H), 4.77 (dd, J=11.9, 3.5 Hz, 1H, 5-H), 4.62 (dd, J=11.9, 4.8 Hz, 1H, 5-H), 4.50 (td, J=4.9, 3.4 Hz, 1H, 4-H), 3.42 (s, 3H, O—CH₃).

### Synthesis of BzABF-STol, 1′-α-S-(4-Methylbenzyl)-2′,3′,5′-O-tribenzoyl-L-arabinofuranose

1-methoxy-2,3,5-tribenzoyl-L-arabinofuranose (5 g, 10.49 mmol) and 4-methylbenzenethiol (1.8 g, 14.69 mmol) were dried together in a desiccator over phosphorous pentoxide under high vacuum overnight, brought under nitrogen and dissolved in anhydrous dichloromethane (73 mL). The solution was cooled to 0° C. and boron trifluoride diethyl etherate (6.6 mL, 52.45 mmol) was added, the solution warmed to room temperature and stirred until no more conversion observed (5.5 hours). The reaction was quenched with triethylamine (13.2 mL), the volatiles were removed under vacuum and the residue taken up in dichloromethane (140 mL). The organic phase was washed with water (140 mL), 75 mM potassium bisulfate solution (2×50 mL), brine (75 mL), dried over magnesium sulfate and evaporated to leave a sticky crude. This was further purified by flash column chromatography on silica gel eluting with hexanes/ethyl acetate to yield a clear, viscous syrup that solidified under high vacuum (68% yield; 5.01 g, 7.14 mmol). ¹H NMR (400 MHz, CDCl₃) δ 8.18-8.13 (m, 2H, Ar—H), 8.08-8.01 (m, 4H, Ar—H), 7.68-7.30 (m, 12H, Ar—H), 7.15 (dt, J=7.9, 0.7 Hz, 1H, Ar—H), 5.79 (dt, J=1.5, 0.7 Hz, 1H, 1-H), 5.74 (t, J=1.5 Hz, 1H, 2-H), 5.68 (ddd, J=4.8, 1.6, 0.9 Hz, 1H, 3-H), 4.91-4.87 (m, 1H, 4-H), 4.83 (dd, J=11.9, 3.6 Hz, 1H, 5-H), 4.76 (dd, J=11.9, 5.2 Hz, 1H, 5-H), 2.35 (s, 3H, Ar—CH₃). LC-MS (ESI): (positive ion) m/z=586.34 [M+H₂O]⁺.

### Synthesis of BzABF-C5

The synthesis of BzABF-C5 is described in Scheme 6.

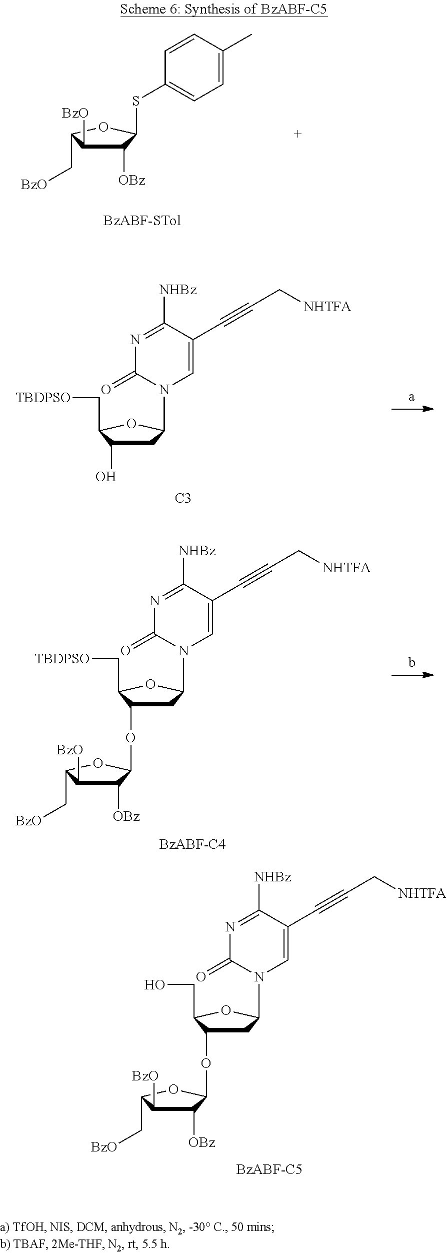

### Synthesis of BzABF-C4, 3′-O-(2″,3″,5″-O-tribenzoyl-α-L-arabinofuranosyl)-5′-O-tertbutyldiphenylsilyl-5-(N-trifluoroacetamido-propargyl)-2′-deoxycytidine

BzABF-STol (1.19 g, 2.086 mmol), known intermediate C3 (1 g, 1.391 mmol) and N-iodosuccinimide (375 mg, 1.669 mmol) were combined in a flask, sealed in a desiccator over phosphorous pentoxide, and dried under high vacuum overnight. The flask was sparged with nitrogen, the reaction components were dissolved in anhydrous dichloromethane (28 mL) and cooled to −30° C. Triflic acid (147 μL, 1.669 mmol) was added slowly, dropwise and the mixture was stirred until no more conversion to product was observed by UPLC. The reaction was quenched with an ice-cold solution of saturated aqueous sodium bicarbonate with 0.1 M sodium thiosulfate (140 mL). The mixture was allowed to warm to room temperature and stirred for 10 minutes. The mixture was then diluted with dichloromethane (100 mL), the organic phase was separated and washed with water (100 mL), the aqueous phase was back extracted with dichloromethane (100 mL), combined organic phases washed with brine and dried over magnesium sulfate. The organics were concentrated under vacuum and purified by flash column chromatography on silica gel, eluting with hexanes/ethyl acetate to give the product as a white solid (58% Yield; 937 mg, 0.805 mmol). ¹H NMR (400 MHz, CDCl₃) δ 8.21 (s, 1H, Ar—H), 8.19-8.13 (m, 2H, Ar—H), 8.01 (ddd, J=9.2, 8.3, 1.3 Hz, 4H, Ar—H), 7.92 (dd, J=8.3, 1.3 Hz, 2H, Ar—H), 7.63-7.23 (m, 20H, Ar—H), 6.56 (s, 1H, N—H), 6.30 (dd, J=7.7, 5.6 Hz, 1H, 1′-H), 5.58-5.52 (m, 1H, 3″-H), 5.39 (d, J=1.5 Hz, 1H, 2″-H), 5.19 (s, 1H, 1″-H), 4.75 (dd, J=11.8, 3.7 Hz, 1H, 5″-HH), 4.63 (dd, J=11.8, 5.2 Hz, 1H, 5″-HH), 4.57 (td, J=4.9, 3.7 Hz, 1H, 4″-H), 4.51-4.45 (m, 1H, 3′-H), 4.21 (t, J=2.6 Hz, 1H, 4′-H), 4.05-4.00 (m, 2H, NC—H₂), 3.97 (dd, J=11.9, 2.9 Hz, 1H, 5′-HH), 3.75 (dd, J=11.8, 2.6 Hz, 1H, 5′-HH), 2.68 (ddd, J=13.7, 5.8, 2.4 Hz, 1H, 2′-H), 2.22 (td, J=14.0, 6.6 Hz, 1H, 2′-H), 0.99 (s, 9H, tBu). LC-MS (ESI): (positive ion) m/z=1164.12 [M+H]⁺, (negative ion) m/z=1162.15 [M−H⁺]⁻.

### Synthesis of BzABF-C5, 3′-O-(2″,3″,5″-O-tribenzoyl-α-L-arabinofuranosyl)-5′-hydroxy-5-(N-trifluoroacetamido-propargyl)-2′-deoxycytidine

BzABF-C4 (570 mg, 0.490 mmol) was dissolved in dry 2-methyltetrahydrofuran (5 mL) under nitrogen, a solution of 1M TBAF (735 μL, 0.735 mmol) in THF was added dropwise and the reaction stirred for 5 hours. The reaction was diluted with ethyl acetate (50 mL) and washed twice with 75 mM potassium bisulfate solution (2×30 mL). The organic was separated, and the aqueous phase was back extracted with ethyl acetate (3×25 mL). The combined organic was concentrated under vacuum and the crude purified by flash column chromatography, eluting carefully with a 30-45% gradient of hexanes/ethyl acetate to remove a co-eluting impurity. Clean fractions are pooled and evaporated to give the desired compound as an off-white/yellow solid (47% yield; 213 mg, 0.230 mmol). ¹H NMR (400 MHz, DMSO) δ 8.07-8.02 (m, 2H, Ar—H), 8.02-7.93 (m, 4H, Ar—H), 7.76-7.68 (m, 2H, Ar—H), 7.66-7.56 (m, 3H, Ar—H), 7.56-7.45 (m, 3H, Ar—H), 7.43-7.35 (m, 2H, Ar—H), 6.18 (t, J=6.3 Hz, 1H, 1′-H), 5.63-5.56 (m, 2H, 2″-H, 3″-H), 5.41 (d, J=1.4 Hz, 1H, 1″H), 5.28 (t, J=5.2 Hz, 1H, 5′O—H), 4.76 (dd, J=11.6, 3.0 Hz, 1H, 5″-HH), 4.72-4.60 (m, 2H, 4″-H, 5″-HH), 4.50 (q, J=4.1 Hz, 1H, 3′-H), 4.22 (s, 2H, NC—H₂), 4.16 (s, 1H, 4′-H), 3.77-3.63 (m, 2H, 5′-H₂), 2.55-2.48 (m, 1H, 2′-HH), 2.46-2.35 (m, 1H, 2′-HH). LC-MS (ESI): (positive ion) m/z=925.70 [M+H]⁺, (negative ion) m/z=923.59 [M−H⁺]⁻.

### Synthesis of dCTP-PA-ABF

The synthesis of dCTP-PA-ABF is described in Scheme 7.

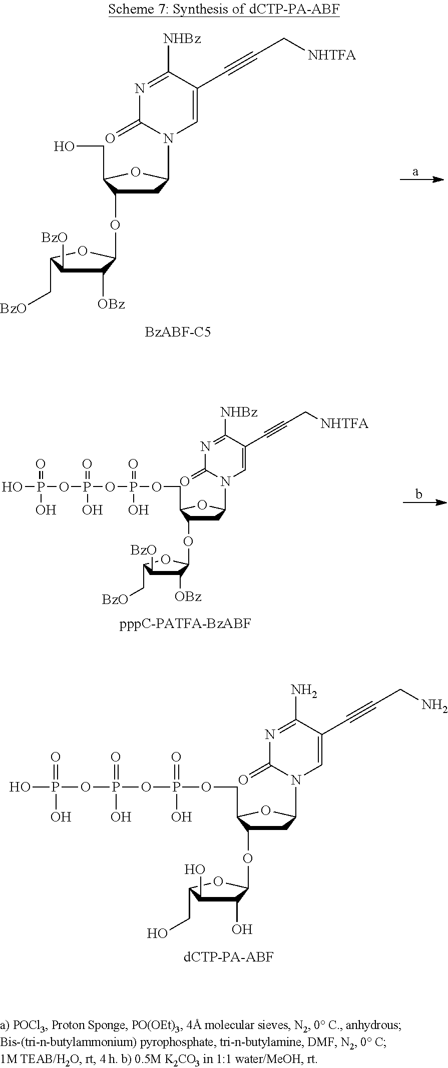

### Synthesis of pppC-PATFA-BzABF, 3′-O-(2″,3″,5″-O-tribenzoyl-α-L-arabinofuranosyl)-5-(N-trifluoroacetamido-propargyl)-2′-deoxycytidine-5′-triphosphate

BzABF-C5 (210 mg, 0.227 mmol), proton sponge (73 mg, 0.341 mmol) and a magnetic stirrer were dried together in a desiccator, over P₂O₅, under high vacuum overnight. Freshly activated 4 Å molecular sieves were added and components brought under nitrogen. Anhydrous triethyl phosphate (1.1 mL) was added, the mixture was cooled to 0° C., freshly distilled POCl₃ (25 L, 0.272 mmol) was added dropwise and the reaction was stirred at 0° C. for 15 minutes. A solution of 0.45M bis-(tri-n-butylammonium) pyrophosphate (2.52 mL, 1.135 mmol) in anhydrous N,N-dimethylformamide was added, followed immediately by anhydrous tri-n-butylamine (216 μL, 0.908 mmol). The mixture was stirred vigorously for 8 minutes at 0° C., before being quenched by an aqueous solution of 2 M TEAB (4 mL). The solution was transferred to a separate flask, the reaction vessel was rinsed with water (4 mL) and the washings added into the 2 M TEAB solution. The combined mixture was then stirred at room temperature for 2.5 hours, before being concentrated under vacuum. The crude product was separated by ion exchange chromatography on DEAE Sephadex, eluting with 0.1 M TEAB/1 M TEAB in 20% acetonitrile in water. The crude triphosphate fractions were concentrated under vacuum and desalted using C18 flash column chromatography, eluting with 0.1M TEAB/acetonitrile. All product-containing fractions were combined, concentrated under reduced pressure and used in the next step without further purification.

### Synthesis of dCTP-PA-ABF, 3′-O-(α-L-arabinofuranosyl)-5-propargylamino-2′-deoxycytidine-5′-triphosphate

The crude triphosphate was dissolved in a solution of 0.5 M potassium carbonate (45 mL) in 1:1 methanol/water and stirred at room temperature until all protecting groups were hydrolysed. The reaction was concentrated under vacuum and passed through a C-18 flash column, eluting with 0.1M TEAB/acetonitrile to remove organic biproducts. The product fractions were concentrated and purified by preparative HPLC to give pure dCTP-PA-ABF (4% yield; 9.50 μmol, determined by UV-Vis spectrometry, λmax=294 nm, ε=8600 M⁻¹ cm⁻¹). LC-MS (ESI): (negative ion) m/z=651.2 [M−H⁺]⁻.

### Synthesis of dATP-PA-ABF

The synthesis of dATP-PA-ABF is described in Scheme 8.

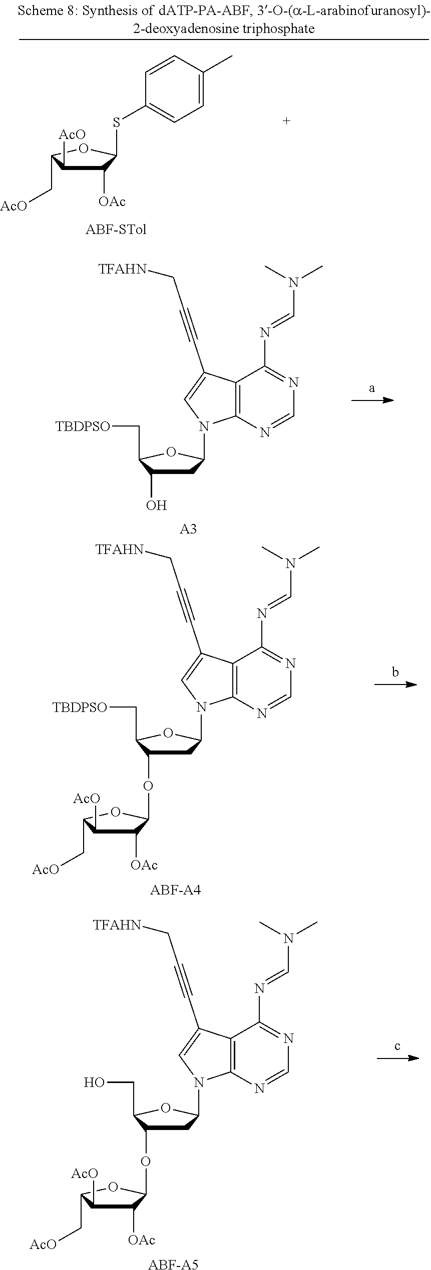

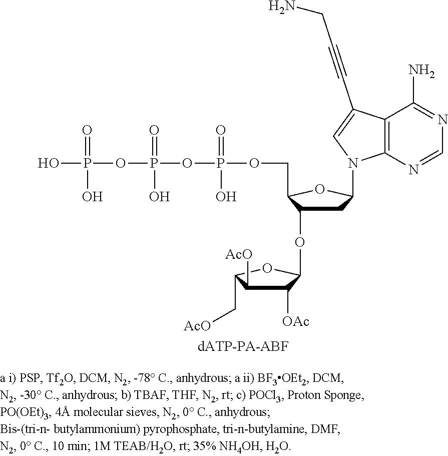

### Synthesis of ABF-A4, 3′-O-(2″,3″,5″-O-triacetoxy-α-L-arabinofuranosyl)-5′-O-tertbutyldiphenylsilyl-7-deaza-7-(N-trifluoroacetamido-propargyl)-2′-deoxyadenosine

ABF-STol (115 mg, 0.30 mmol) and 1-(phenylsulfinyl)piperidine (75 mg, 0.36 mmol) were combined in a round bottomed flask and dried under high vacuum for 3 hours. The components were dissolved in dry dichloromethane (3 mL) under nitrogen, cooled to −78° C., triflic anhydride (58 μL, 0.17 mmol) was added slowly, dropwise and mixture stirred for 5 minutes. Known intermediate A3 (208 mg, 0.30 mmol) was dried under high vacuum for 3 hours and dissolved in dichloromethane (1 mL) under nitrogen. The A3 solution was added slowly, dropwise to the stirring solution of activated ABF-STol, followed immediately by boron trifluoride diethyl etherate (37 μL, 0.15 mmol). The combined mixture was stirred at −78° C. for 15 minutes, before being warmed to −30° C. and stirred for a further hour. The reaction was quenched with triethylamine (100 μL) and methanol (3 mL) at −30° C., before being concentrated under reduced pressure and purified by flash column chromatography on silica gel, eluting in dichloromethane/ethyl acetate to give the desired product. 37% yield (133 mg, 0.11 mmol), 47% yield when combining impure fractions. ¹H NMR (400 MHz, CDCl₃) δ 8.71 (s, 1H, C-Hf), 8.36 (s, 1H, 2-H), 7.64-7.55 (m, 5H, Ar—H), 7.49 (s, 1H, H-8), 7.40-7.26 (m, 5H, Ar—H), 6.59 (dd, J=7.5, 6.0 Hz, 1H, 1′-H), 6.56-6.45 (brs, 1H, N—H), 5.09 (s, 1H, 1″-H), 5.03 (dd, J=1.7, 0.6 Hz, 1H, 2″-H), 4.96 (dd, J=5.1, 1.7 Hz, 1H, 3″-H), 4.53 (dt, J=6.3, 3.2 Hz, 1H, 3′-H), 4.34 (dd, J=11.5, 3.3 Hz, 1H, 5′-HH), 4.29 (d, J=5.1 Hz, 2H, NC—H₂), 4.26-4.13 (m, 2H, 4″-H, 5″-HH), 4.10 (q, J=3.8 Hz, 1H, 4′-H), 3.80-3.70 (m, 2H, 5′-H), 3.16 (d, J=0.6 Hz, 3H, NC—H₃), 3.15 (s, 3H, NC—H₃) 2.59-2.37 (m, 2H, 2′-H₂), 2.08 (s, 3H, OC—H₃), 2.03 (s, 3H, OC—H₃), 2.01 (s, 3H, OC—H₃), 1.02 (s, 9H, tBu).

### Synthesis of ABF-A5, 3′-O-(2″,3″,5″-O-triacetoxy-α-L-arabinofuranosyl)-5′-hydroxy-7-deaza-7-(N-trifluoroacetamido-propargyl)-2′-deoxyadenosine

ABF-A5 was synthesized from ABF-A4 (128 mg, 0.13 mmol) via the procedure outlined in the synthesis of BzABF-C5 (Scheme 6). 40% yield (42 mg, 52 μmol). ¹H NMR (400 MHz, CDCl₃) δ 8.72 (s, 1H, N—H), 8.33 (s, 1H, 2-H), 7.22 (s, 1H, N—H), 6.92 (s, 1H, N—H), 6.08 (dd, J=9.4, 5.6 Hz, 1H, 1′-H), 5.11 (s, 1H, 1″-H), 5.04 (d, J=1.8 Hz, 1H, 2″-H), 4.96 (dd, J=5.3, 1.8 Hz, 1H, 3″-H), 4.63 (d, J=5.7 Hz, 1H, 3′-H), 4.34 (dd, J=12.5, 4.0 Hz, 3H, NC—H₂, 5″-HH), 4.26-4.14 (m, 3H, 4′-H, 4″-H, 5″-HH), 3.96-3.88 (m, 1H, 5′-HH), 3.74-3.64 (m, 1H, 5′-HH), 3.13 (d, J=8.9 Hz, 6H, N(C—H₃)₂), 2.99 (ddd, J=14.4, 9.4, 5.8 Hz, 1H, 2′-HH), 2.32 (dd, J=13.6, 5.7 Hz, 1H, 2′-HH), 2.06 (d, J=2.5 Hz, 6H, OC—H₃, OC—H₃), 2.03 (s, 3H, OC—H₃).

### Synthesis of dATP-PA-ABF, 3′-O-(α-L-arabinofuranosyl)-2-deoxyadenosine triphosphate

dATP-PA-ABF was synthesized from ABF-A4 (120 mg, 0.165 mmol) via the procedure outlined in the synthesis of (2) 3′-ABF-TTP. Triphosphate obtained as a white solid. 35% yield (58 μmol). LC-MS (ESI): (negative ion) m/z=674.1 [M−H⁺]⁻.

### Synthesis of ABF-dG4

The synthesis of ABF-dG4 is described in Scheme 9.

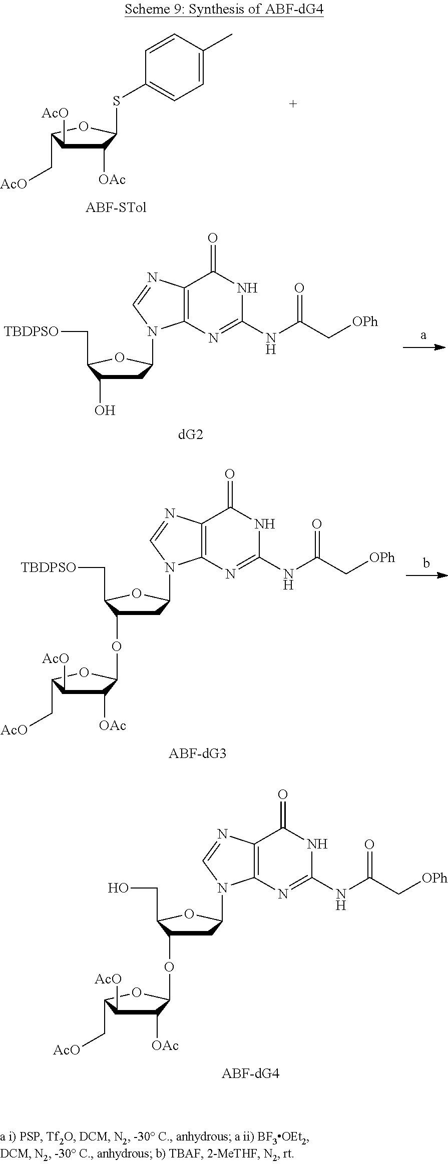

### Synthesis of ABF-dG3, 3′-O-(2″,3″,5″-O-triacetoxy-α-L-arabinofuranosyl)-5′-O-tertbutyldiphenylsilyl-N-(2-phenoxyacetamido)-2′-deoxyguanosine

ABF-STol (598 mg, 1.563 mmol) and 1-(phenylsulfinyl)piperidine (392 mg, 1.87 mmol) were combined in a first round bottomed flask. Known intermediate dG2 (1.0 g, 1.563 mmol) was sealed in a second round bottomed flask. Both the first and second round bottomed flasks were dried in a desiccator, over P₂O₅, under high vacuum overnight. The flask containing ABF-STol was sealed under nitrogen, dissolved in anhydrous dichloromethane (31 mL), cooled to −30° C., and stirred for 15 minutes. Triflic anhydride (289 μL) was added slowly, dropwise, and reaction stirred for 5 minutes. The previously dried dG2 was sealed under nitrogen and suspended in minimal ice-cold dichloromethane, and added dropwise to the activated ABF-STol, along with simultaneous, dropwise addition of boron trifluoride diethyl etherate (197 μL, 1.563 mL). Additional dichloromethane washes of the dG2 suspension were added until all the dG2 material was added, the combined reaction solution was stirred at −30° C. for 35 minutes. The reaction was quenched with a cold, 0.25 M solution of sodium bicarbonate in 1:1 methanol/water (100 mL), warmed to room temperature and stirred for 10 minutes. The organic layer was separated, and the aqueous layer extracted twice with dichloromethane (2×50 mL), the combined organic was dried over magnesium sulfate, filtered and concentrated under vacuum. The crude dry loaded onto a silica column using 10% methanol in dichloromethane with 0.1% triethylamine as an additive and purified by flash column chromatography. The column was eluted with 0.1% triethylamine in dichloromethane, and 10% methanol in dichloromethane with 0.1% triethylamine to give the desired product. 50% yield. ¹H NMR (400 MHz, CDCl₃) δ 11.80 (s, 1H, N—H), 9.12 (s, 1H, N—H), 7.84 (s, 1H, 8-H), 7.62 (ddt, J=6.6, 3.0, 1.5 Hz, 4H, Ar—H), 7.46-7.40 (m, 2H, Ar—H), 7.39-7.33 (m, 6H, Ar—H), 7.10 (tt, J=7.4, 1.2 Hz, 1H, Ar—H), 7.01-6.96 (m, 2H, Ar—H), 6.24 (t, J=6.8 Hz, 1H, 1′-H), 5.17 (s, 1H, 1″-H), 5.12 (dd, J=1.8, 0.6 Hz, 1H, 2″-H), 5.03 (ddd, J=5.2, 1.9, 0.6 Hz, 1H, 3″-Hz), 4.69 (s, 2H, OC—H₂), 4.63 (dd, J=7.1, 4.0 Hz, 1H, 3′-H), 4.43 (dd, J=11.6, 3.6 Hz, 1H, 5″-HH), 4.32 (td, J=5.4, 3.6 Hz, 1H, 4″-H), 4.24 (dd, J=11.6, 5.8 Hz, 1H, 5″-H), 4.21-4.16 (m, 1H, 4′-H), 3.84-3.73 (m, 2H, 5′-Hz), 2.61-2.53 (m, 2H, 2′-H₂), 2.11 (s, 6H, OC—H₃, OC—H₃), 2.07 (s, 3H, OC—H₃), 1.05 (s, 9H, tBu). LC-MS (ESI): (positive ion) m/z=868.65 [M+H]⁺, (negative ion) m/z=896.67 [M−H⁺]⁻.

### Synthesis of ABF-dG4, 3′-O-(2″,3″,5″-O-triacetoxy-α-L-arabinofuranosyl)-5′-hydroxy-N-(2-phenoxyacetamido)-2′-deoxyguanosine

ABF-dG3 (400 mg, 0.445 mmol) was dried under high vacuum for 1 hour, sealed under nitrogen, dissolved in dry 2-methyltetrahydrofuran (4.5 mL) and cooled to 0° C. 1M tetra-N-butylammonium fluoride solution in tetrahydrofuran (668 μL, 0.668 mmol) was added slowly dropwise and reaction stirred at 0° C. overnight. The reaction was diluted with ethyl acetate (45 mL) and extracted three times with saturated ammonium chloride solution (3×45 mL). The combined aqueous phases were back-extracted 3 times with ethyl acetate (3×90 mL), the organic phases were combined and washed with brine (50 mL), dried over magnesium sulfate and concentrated under vacuum. The resulting crude was purified by flash column chromatography on silica gel, eluting with dichloromethane/20% methanol in dichloromethane, to yield the product as an off-white solid after drying under high vacuum overnight. 77% yield (0.226 mg, 0.342 mmol). ¹H NMR (400 MHz, DMSO) δ 11.80 (s, 2H, N—H, N—H), 8.28 (s, 1H, 8-H), 7.37-7.28 (m, 2H, Ar—H), 7.04-6.94 (m, 3H, Ar—H), 6.21 (dd, J=7.8, 5.9 Hz, 1H, 1′-H), 5.28 (s, 1H, 1″-H), 5.06 (t, J=5.4 Hz, 1H, 5′O—H), 5.00-4.95 (m, 2H, 2″-H, 3″-H), 4.87 (s, 2H, OC—H₂), 4.51 (dt, J=5.9, 2.8 Hz, 1H, 3′-H), 4.36 (dd, J=11.9, 3.2 Hz, 1H, 5″-HH), 4.29 (td, J=5.2, 3.1 Hz, 1H, 4″-H), 4.16 (dd, J=11.9, 5.2 Hz, 1H, 5″-HH), 4.03 (tt, J=5.1, 2.2 Hz, 1H, 4′-H), 3.61-3.48 (m, 2H, 5′-H₂), 2.83-2.73 (m, 1H, 2′-HH), 2.49-2.42 (m, 1H, 2′-HH), 2.10 (s, 3H, OC—H₃), 2.09 (s, 2H, OC—H₃), 2.04 (s, 3H, OC—H₃). LC-MS (ESI): (positive ion) m/z=660.56 [M+H]⁺, (negative ion) m/z=658.58 [M−H⁺]⁻.

### Synthesis of dGTP-ABF

The synthesis of dGTP-ABF is described in Scheme 10.

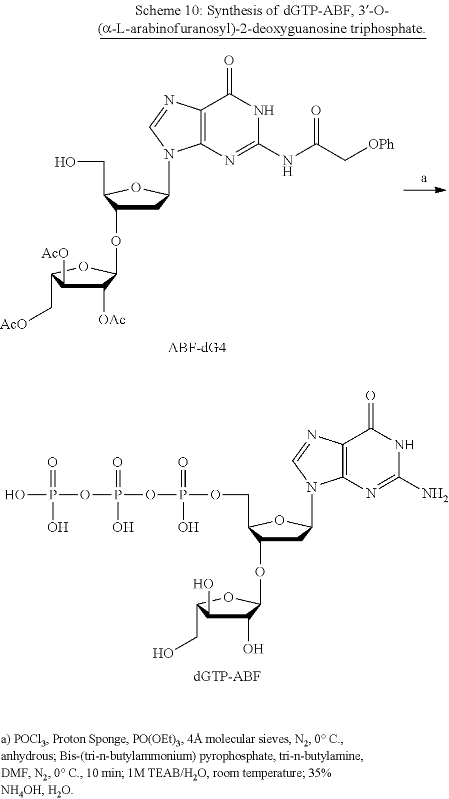

### Synthesis of dGTP-ABF, 3′-O-(α-L-arabinofuranosyl)-2-deoxyguanosine Triphosphate

dGTP-ABF was synthesized from ABF-dG4 (225 mg, 0.340 mmol) via the procedure outlined in the synthesis of (2) 3′-ABF-TTP. Triphosphate obtained as a white solid (32% yield; 108 μmol, determined by UV-Vis spectrometry, λmax=253 nm, ε=13700 M⁻¹ cm⁻¹). LC-MS (ESI): (negative ion) m/z=638.30 [M−H⁺]⁻.

### Example 2. Synthesis of 3′-glycosyl Nucleosides and Nucleotides

### General Procedure for the Synthesis of 5′-tertbutyldiphenylsilyl 3′-[D-per(O-benzoyl)glycopyranosyl]thymidine

5′-tertbutyldiphenylsilyl thymidine (0.625 mmol), tolyl per(O-benzoyl)-1-thio-(β)-D-glycopyranoside (0.937 mmol), N-iodosuccinimide (1.0 mmol) and activated 4 Å molecular sieves were added to a dry flask and dissolved in anhydrous dichloromethane (6 mL) under nitrogen gas. The suspension was stirred for 10 minutes at room temperature then cooled to −30° C. Trifluoromethanesulfonic acid (66 uL, 0.75 mmol) was added slowly dropwise and the solution turned deep red/brown. The reaction was stirred at temperature <−5° C. for 3 hours, then quenched with 10 mL of saturated aq. NaHCO₃ at −10° C., then with 10 mL of saturated aqueous Na₂S₂O₃ and stirred until it became clear. The mixture was diluted with 50 mL of dichloromethane, the aqueous phase was separated and the organic phase was washed with 100 mL of saturated aq. NaHCO₃ and 100 mL of brine. The organic phase was dried over MgSO₄, filtered and evaporated under reduced pressure. The crude was purified by purified by flash chromatography on silica gel (gradient of petroleum ether/ethyl acetate from 8:2 to 2:8).

### 5′-tertbutyldiphenylsilyl 3′-[(β)-D-2,3,4,6-(tetra-O-benzoyl)glucopyranosyl]thymidine

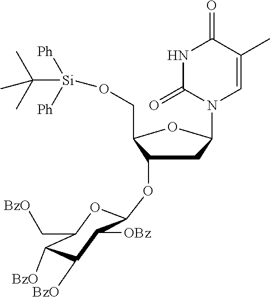

Yield: 500 mg (0.472 mmol), 75%. ¹H NMR (400 MHz, CDCl₃) δ (ppm) 8.51 (s, 1H, NH), 7.95-7.72 (m, 8H, Ar), 7.53 (ddt, J=11.1, 6.6, 1.5 Hz, 4H, Ar), 7.48-7.11 (m, 18H, Ar), 6.12 (dd, J=7.7, 6.2 Hz, 1H, 1′-CH), 5.78 (t, J=9.7 Hz, 1H, 3-CH glu), 5.51 (t, J=9.7 Hz, 1H, 4-CH glu), 5.39 (dd, J=9.8, 7.9 Hz, 1H, 2-CH glu), 4.67 (d, J=7.9 Hz, 1H, 1-CH glu), 4.51 (dd, J=12.2, 3.2 Hz, 1H, 6-CH glu), 4.44-4.37 (m, 1H, 6-CH glu), 4.37-4.30 (m, 1H, 3′-CH), 4.09-3.96 (m, 1H, 5-CH glu), 3.90-3.72 (m, 2H, 4′-CH, 5′-CH), 3.58 (dd, J=11.4, 2.5 Hz, 1H, 5′-CH), 2.50 (ddd, J=14.0, 6.2, 2.9 Hz, 1H, 2′-CH), 2.01 (dt, J=14.6, 7.5 Hz, 1H, 2′CH), 1.55 (d, J=1.2 Hz, 3H, CH₃—Ar), 0.98 (s, 9H, tBu). LC-MS (ESI): (positive ion) m/z 1059 (M+H⁺), 1076 (M+NH₄⁺).

### 5′-tertbutyldiphenylsilyl 3′-[(3)-D-2,3,4-(tri-O-benzoyl)xylopyranosyl]thymidine

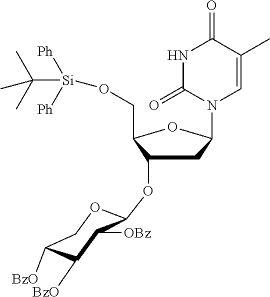

Yield: 452 mg (0.48 mmol), 78%. ¹H NMR (400 MHz, CDCl₃): δ (ppm) 8.21 (s, 1H, NH), 7.92 (ddd, J=15.1, 8.4, 1.4 Hz, 4H, Bz), 7.84 (dd, J=8.4, 1.3 Hz, 2H, Bz), 7.56 (ddd, J=8.0, 6.6, 1.5 Hz, 4H, Ar), 7.51-7.28 (m, 14H, Ar), 7.26-7.20 (m, 2H, Ar), 6.21 (dd, J=8.1, 5.8 Hz, 1H, 1′-CH), 5.65 (t, J=7.1 Hz, 1H, 3-CH xyl), 5.25 (dd, J=7.1, 5.3 Hz, 1H, 2-CH xyl), 5.21 (td, J=6.9, 4.2 Hz, 1H, 4-CH xyl), 4.74 (d, J=5.3 Hz, 1H, 1-CH xyl), 4.45 (dt, J=6.7, 2.6 Hz, 1H, 3′-CH), 4.34 (dd, J=12.2, 4.2 Hz, 1H, 5-CHH xyl), 3.92 (q, J=2.9 Hz, 1H, 4′-CH), 3.82 (dd, J=11.5, 3.3 Hz, 1H, 5′-CHH), 3.66 (dd, J=11.5, 2.7 Hz, 1H, 5′-CHH), 3.60 (dd, J=12.2, 6.8 Hz, 1H, 5-CHH xyl), 2.53 (ddd, J=13.8, 5.8, 2.4 Hz, 1H, 2′-CHH), 2.23-2.07 (m, 1H, 2′-CHH), 1.56 (d, J=1.2 Hz, 3H, CH₃), 1.01 (s, 9H, tBu). LC-MS (ESI): (positive ion) m/z 925 (M+H⁺), 942 (M+NH₄⁺); (negative ion) m/z 923 (M⁻).

### 5′-tertbutyldiphenylsilyl 3′-[(α)-D-2,3,4,6-(tetra-O-benzoyl)mannopyranosyl]thymidine

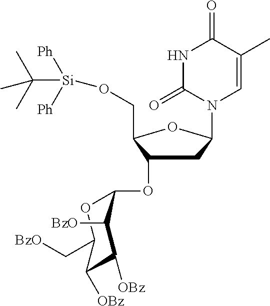

Yield: 263 mg (0.248 mmol), 40%. ¹H NMR (400 MHz, CDCl₃): δ (ppm) 8.09 (s, 1H, NH), 8.04-7.91 (m, 4H, Bz), 7.90-7.85 (m, 2H, Bz), 7.76 (dd, J=8.4, 1.4 Hz, 2H, Bz), 7.61-7.49 (m, 5H, Ar), 7.47-7.19 (m, 18H, Ar), 6.38 (dd, J=8.7, 5.5 Hz, 1H, 1′-CH), 6.06 (t, J=10.0 Hz, 1H, 4-CH man), 5.79 (dd, J=10.2, 3.3 Hz, 1H, 3-CH man), 5.60 (dd, J=3.3, 1.8 Hz, 1H, 2-CH man), 5.05 (d, J=1.8 Hz, 1H, 1-CH man), 4.60-4.53 (m, 1H, 3′-CH man), 4.48 (dd, J=12.1, 2.6 Hz, 1H, 6-CH man), 4.37-4.24 (m, 3H, 6-CH man, 5-CH man, 4′-CH), 3.91-3.73 (m, 2H, 5′-CH₂), 2.53 (ddd, J=13.8, 5.6, 1.6 Hz, 1H, 2′-CHH), 2.24-2.07 (m, 1H, 2′-CHH), 1.54 (d, J=1.2 Hz, 3H, CH₃), 1.00 (s, 9H, tBu). LC-MS (ESI): (positive ion) m/z 1059 (M+H⁺), 1076 (M+NH₄⁺), 1082 (M+Na⁺).

### General Procedure for the Synthesis of 3′-[D-per(O-benzoyl)glycopyranosyl]thymidine

The 5′-TBDPS 3′-glycosyl thymidine was dissolved in 10 mL of anhydrous tetrahydrofuran under nitrogen, then 1.2 equivalents of tetrabutylammonium fluoride (1.0 M solution in THF) were added. The reaction was stirred at room temperature until complete, then it was diluted with ethyl acetate and washed first with 1 M NaH₂PO₄ (pH ˜4), then saturated aq. NaHCO₃. The organic phase was dried over MgSO₄, filtered and evaporated under reduced pressure. The crude was purified by purified by flash chromatography on silica gel (gradient of petroleum ether/ethyl acetate from 8:2 to 100% ethyl acetate).

### 3′-[(β)-D-2,3,4,6-(tetra-O-benzoyl)glucopyranosyl]thymidine

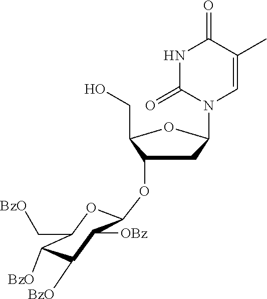

Yield: 450 mg, (0.548 mmol), 96%. ¹H NMR (400 MHz, CDCl₃): δ (ppm) 8.74 (s, 1H, NH), 7.96-7.88 (m, 2H, Bz), 7.91-7.79 (m, 2H, Bz), 7.79-7.72 (m, 2H, Bz), 7.49-7.39 (m, 3H, Bz), 7.38-7.24 (m, 7H, Bz), 7.23-7.16 (m, 2H, Bz), 7.01 (d, J=1.3 Hz, 1H, H-6 T), 5.84 (t, J=9.7 Hz, 1H, 3-CH glu), 5.76 (t, J=6.8 Hz, 1H, 1′-CH), 5.57 (t, J=9.7 Hz, 1H, 4-CH glu), 5.44 (dd, J=9.8, 7.9 Hz, 1H, 2-CH glu), 4.90 (d, J=7.9 Hz, 1H, 1-CH glu), 4.62-4.43 (m, 3H, 3′-CH and 6-CH₂ glu), 4.12 (ddd, J=9.8, 5.7, 3.2 Hz, 1H, 5-CH glu), 3.83 (dt, J=4.5, 2.6 Hz, 1H, 4′-CH), 3.66 (dd, J=12.2, 2.7 Hz, 1H, 5′-CHH), 3.44 (dd, J=12.2, 2.7 Hz, 1H, 5′-CHH), 2.58-2.35 (m, 2H, 2′-CH₂), 1.78 (d, J=1.2 Hz, 3H, CH₃). LC-MS (ESI): (positive ion) m/z 821 (M+H⁺), 843 (M+Na⁺). (negative ion) m/z 819 (M⁻).

### 3′-[(β)-D-2,3,4-(tri-O-benzoyl)xylopyranosyl]thymidine

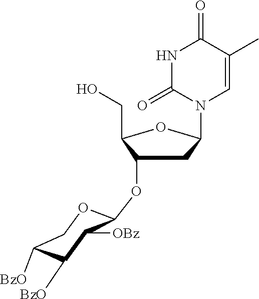

Yield: 242 mg, (0.352 mmol), 82%. ¹H NMR (400 MHz, CDCl₃): δ (ppm) 8.37 (s, 1H, NH), 7.97-7.83 (m, 6H, Bz), 7.53-7.40 (m, 3H, Bz), 7.37-7.25 (m, 7H, Bz), 7.16 (d, 1H, J=1.3 Hz, H-6), 5.89 (t, J=6.8 Hz, 1H, 1′-CH), 5.70 (t, J=7.5 Hz, 1H, 3-CH xyl), 5.30 (dd, J=7.6, 5.7 Hz, 1H, 2-CH xyl), 5.24 (td, J=7.4, 4.4 Hz, 1H, 4-CH xyl), 4.86 (d, J=5.7 Hz, 1H, 1-CH xyl), 4.52 (dt, J=6.9, 3.8 Hz, 1H, 3′-CH), 4.35 (dd, J=12.1, 4.4 Hz, 1H, 5-CHH xyl), 3.92 (dt, J=3.7, 2.7 Hz, 1H, 4′-CH), 3.75 (dd, J=12.1, 2.7 Hz, 1H, 5′-CHH), 3.65 (dd, J=12.1, 7.3 Hz, 1H, 5-CHH xyl), 3.56 (dd, J=12.1, 2.8 Hz, 1H, 5′-CHH), 2.54-2.40 (m, 2H, 2′-CH₂), 1.83 (d, J=1.2 Hz, 3H, CH₃). LC-MS (ESI): (positive ion) m/z 687 (M+H⁺), 708 (M+Na⁺); (negative ion) m/z 685 (M⁻).

### 3′-[(α)-D-2,3,4,6-(tetra-O-benzoyl)mannopyranosyl]thymidine

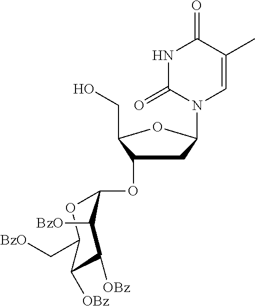

Yield: 146 mg, (0.178 mmol), 74%. ¹H NMR (400 MHz, CDCl₃): δ (ppm) 8.27 (s, 1H, NH), 8.02-7.95 (m, 4H, Bz), 7.94-7.87 (m, 2H, Bz), 7.81-7.73 (m, 2H, Bz), 7.59-7.41 (m, 3H, Bz), 7.40-7.26 (m, 8H, Bz), 7.25-7.16 (m, 2H, Bz, H-6), 6.10 (t, J=7.0 Hz, 1H, 1′-CH), 6.02 (t, J=10.1 Hz, 1H, 4-CH man), 5.80 (dd, J=10.2, 3.4 Hz, 1H, 3-CH man), 5.61 (dd, J=3.4, 1.8 Hz, 1H, 2-CH man), 5.14 (d, J=1.8 Hz, 1H, 1-CH man), 4.70-4.58 (m, 2H, 3′-CH and 6-CHH man), 4.47 (dd, J=12.1, 4.8 Hz, 1H, 6-CHH man), 4.37 (ddd, J=10.1, 4.9, 3.1 Hz, 1H, 5-CH man), 4.24 (q, J=2.9 Hz, 1H, 4′-CH man), 3.84 (dd, J=12.1, 2.8 Hz, 1H, 5′-CHH), 3.74 (dd, J=12.1, 2.9 Hz, 1H, 5′-CHH), 2.53-2.37 (m, 2H, 2′-CH₂), 1.85 (d, J=1.2 Hz, 3H, CH₃). LC-MS (ESI): (positive ion) m/z 821 (M+H⁺), 843 (M+Na⁺); (negative ion) m/z 819 (M⁻).

### General Procedure for the Synthesis of 3′-[(β)-D-Glycopyranosyl]Thymidine

The 3′-glycosyl thymidine was dissolved in 0.5 mL of methanol and 2.5 mL of ammonium hydroxide (35% aq.) was added. The reaction was stirred at room temperature until complete. The volatiles were removed under reduced pressure, then the residue was suspended in 0.1 M aqueous triethylammonium bicarbonate (TEAB)/acetonitrile, filtered and purified by preparative HPLC using a YMC-Pack-Pro C18 column, eluting with 0.1 M TEAB and acetonitrile.

### 3′-[(β)-D-glucopyranosyl]thymidine

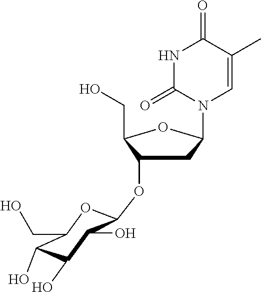

Yield: 32 μmols. ¹H NMR (400 MHz, D₂O): δ (ppm) 7.55 (s, 1H, H-6 T), 6.21 (dd, J=7.6, 6.2 Hz, 1H, 1′-CH), 4.51 (q, J=6.6, 3.3 Hz, 1H, 3′-CH), 4.47 (d, J=7.9 Hz, 1H, 1-CH glu), 4.12 (dt, J=5.0, 3.7 Hz, 1H, 4′-CH), 3.83 (dd, J=12.4, 2.2 Hz, 1H, 6-CHH glu), 3.79-3.67 (m, 2H, 5′-CH₂), 3.64 (dd, J=12.4, 5.9 Hz, 1H, 6-CHH), 3.44-3.35 (m, 2H, 4-CH glu, 5-CH glu), 3.31 (dd, J=9.8, 8.9 Hz, 1H, 3-CH glu), 3.22 (dd, J=9.3, 7.9 Hz, 1H, 2-CH glu), 2.50 (ddd, J=14.4, 6.3, 3.3 Hz, 1H, 2′-CHH), 2.33 (ddd, J=14.3, 7.7, 6.6 Hz, 1H, 2′-CHH), 1.81 (d, J=1.2 Hz, 3H, CH₃). LC-MS (ESI): (positive ion) m/z 405 (M+H⁺), 427 (M+Na⁺); (negative ion) m/z 403 (M⁻).

### 3′-[(β)-D-xylopyranosyl]thymidine

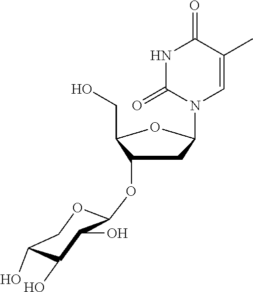

Yield: 37 μmols (74%). ¹H NMR (400 MHz, D₂O): δ (ppm) 7.54 (q, J=1.2 Hz, 1H, H-6 T), 6.19 (dd, J=7.5, 6.3 Hz, 1H, 1′-CH), 4.46 (dt, J=6.7, 3.4 Hz, 1H, 3′-CH), 4.41 (d, J=7.8 Hz, 1H, 1-CH xyl), 4.10 (dt, J=4.9, 3.8 Hz, 1H, 4′-CH), 3.89 (dd, J=11.6, 5.4 Hz, 1H, 5-CHH), 3.76 (dd, J=12.4, 4.0 Hz, 1H, 5′-CHH), 3.70 (dd, J=12.4, 4.9 Hz, 1H, 5′-CHH), 3.54 (ddd, J=10.5, 9.1, 5.5 Hz, 1H, 4-CH xyl), 3.37 (t, J=9.2 Hz, 1H, 3-CH xyl), 3.25 (t, J=11.6, 10.6 Hz, 1H, 5-CHH xyl), 3.20 (dd, J=9.3, 7.9 Hz, 1H, 2-CH xyl), 2.45 (ddd, J=14.3, 6.3, 3.4 Hz, 1H, 2′-CHH), 2.32 (dt, J=14.3, 7.2 Hz, 1H, 2′-CHH), 1.81 (d, J=1.2 Hz, 3H, CH₃). LC-MS (ESI): (positive ion) m/z 375 (M+H⁺), 387 (M+Na⁺); (negative ion) m/z 373 (M⁻).

### 3′-[(α)-D-mannopyranosyl]thymidine

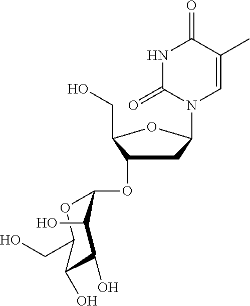

Yield: 32 μmols, 77%. ¹H NMR (400 MHz, D₂O): δ (ppm) 7.55 (q, J=1.2 Hz, 1H, H-6 T), 6.31-6.14 (m, 1H, 1′-CH), 4.91 (d, J=1.8 Hz, 1H, 1-CH man), 4.45 (dt, J=6.5, 3.2 Hz, 1H, 3′-CH), 4.12 (dt, J=4.8, 3.7 Hz, 1H, 4′-CH), 3.90 (dd, J=3.4, 1.8 Hz, 1H, 2-CH man), 3.82 (dd, J=12.2, 1.9 Hz, 1H, 5-CHH man), 3.79-3.65 (m, 4H, 6-CHH man, 3-CH man, 5′-CH₂), 0.62-3.52 (m, 2H, 5-CHH man, 6-CHH man), 2.44 (ddd, J=14.2, 6.3, 3.1 Hz, 1H, 2′-CH₂), 2.32-2.21 (m, 1H, 2′-CH₂), 1.82 (d, J=1.2 Hz, 3H, CH₃). LC-MS (ESI): (positive ion) m/z 405 (M+H⁺), 427 (M+Na⁺); (negative ion) m/z 403 (M⁻).

### General Procedure for the Synthesis of 5′-triphosphate 3′-[(β)-D-glycopyranosyl]thymidine

The nucleoside (200 mg, ˜0.25 mmol,) was dried under reduced pressure over P₂O₅ for 18 hrs. Anhydrous triethyl phosphate (4 mL) and some freshly activated 4 Å molecular sieves were added to it under nitrogen, then the reaction flask was cooled to 0° C. in an ice-bath. Freshly distilled POCl₃ (31 μL, ˜0.3 mmoles) was added dropwise followed by Proton Sponge® (80 mg, 0.35 mmol). After the addition, the reaction was further stirred at 0° C. for 15 minutes. Then, a 0.5 M solution of pyrophosphate as bis-tri-n-butylammonium salt (2.5 mL, 1.25 mmol) in anhydrous DMF was quickly added, followed immediately by tri-n-butyl amine (270 μL, 1.1 mmol). The reaction was kept in the ice-water bath for another 10 minutes, then quenched by pouring it into 1 M aqueous triethylammonium bicarbonate (TEAB, 10 mL) and stirred at room temperature for 4 hours. All the solvents were evaporated under reduced pressure. A 35% aqueous solution of ammonia (10 mL) was added to the above residue and the mixture was stirred at room temperature until complete removal of all the protecting groups. The solvents were then evaporated under reduced pressure. The crude product was purified firstly by ion-exchange chromatography on DEAE-Sephadex A25 (100 g). The column was eluted with a linear gradient of aqueous triethylammonium bicarbonate (TEAB, from 0.1 M to 1 M). The fractions containing the triphosphate were pooled and the solvent was evaporated to dryness under reduced pressure. The crude material was further purified by preparative scale HPLC using a YMC-Pack-Pro C18 column, eluting with 0.1 M TEAB and acetonitrile. The nucleotide triphosphate was obtained as triethylammonium salt.

### 5′-triphosphate 3′-[(β)-D-glucopyranosyl]thymidine

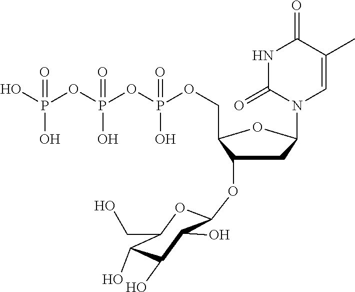

Yield: 112 μmol (45%), based on ε₂₆₇=9600 M⁻¹ cm⁻¹. ¹H NMR (400 MHz, D₂O): δ (ppm) 7.68 (d, J=1.5 Hz, 1H, H-6 T), 6.28 (dd, J=8.4, 5.8 Hz, 1H, 1′-CH), 4.57 (d, J=7.9 Hz, 1H, 1-CH glu), 4.30 (p, J=3.1 Hz, 1H, 4′-CH), 4.15 (qdd, J=11.5, 7.3, 4.3 Hz, 2H, 5′-CH₂), 3.84 (dd, J=12.5, 2.3 Hz, 1H, 6-CHH glu), 3.63 (dd, J=12.5, 5.7 Hz, 1H, 6-CHH glu), 3.52-3.41 (m, 2H, 3-CH glu, 5-CH glu), 3.30 (dd, J=11.1, 7.9 Hz, 1H, 4-CH glu), 3.22-3.16 (m, 1H, 2-CH glu), 2.50 (ddd, J=14.3, 6.0, 2.3 Hz, 1H, 2′-CHH), 2.34 (ddd, J=14.4, 8.6, 6.2 Hz, 1H, 2′-CHH), 1.85 (s, 3H, CH₃). LC-MS (ESI): (negative ion) m/z 643 (M⁻).

### 5′-triphosphate 3′-[(β)-D-xylopyranosyl]thymidine

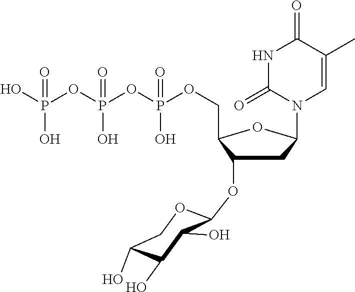

Yield: 133 μmols (47%), based on ε₂₆₇=9600 M⁻¹ cm⁻¹. ¹H NMR (400 MHz, D₂O): δ ppm 7.67 (q, J=1.1 Hz, 1H, H-6 T), 6.27 (dd, J=8.4, 5.9 Hz, 1H, 1′-CH), 4.65 (dt, J=5.3, 2.3 Hz, 1H, 3′-CH), 4.51 (d, J=7.9 Hz, 1H, 1-CH xyl), 4.29 (h, J=2.2, 1.7 Hz, 1H, 4′-CH), 4.15 (pt, J=7.9, 4.3 Hz, 2H, 5′-CH₂), 3.88 (dd, J=11.6, 5.3 Hz, 1H, 5-CHH), 3.52 (m, 1H, 4-CH), 3.42 (t, J=9.2 Hz, 1H, 3-CH), 3.31 (dd, J=11.6, 10.5 Hz, 1H, 5-CHH), 3.19-3.15 (m, 1H, 2-CH), 2.44 (ddd, J=14.2, 6.0, 2.4 Hz, 1H, 2′-CHH), 2.33 (ddd, J=14.4, 8.5, 6.1 Hz, 1H, 2′-CHH), 1.85 (d, J=1.2 Hz, 3H, CH₃). LC-MS (ESI): (negative ion) m/z 613 (M⁻).

### Example 3. 3′-ABF Blocking Group Stability Testing

T Nucleoside was blocked with a 3′-O-α-L-arabinofuranoside (3′-ABF,

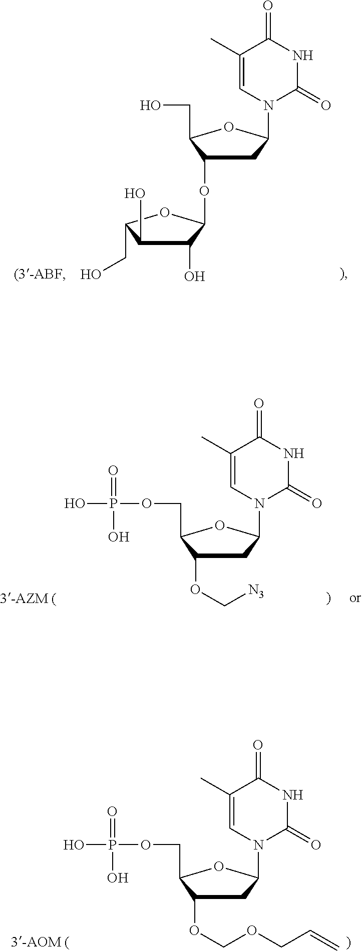

blocking groups. 1 mL of 3′ blocked T nucleosides at 0.1 mM were made in a solution of 50 mM glycine buffer (pH 9.9), 50 mM NaCl, and 1 mM EDTA. F

These solutions were incubated at 60° C. in a heating block for 1 month. Spontaneous cleavage of the 3′-ABF under incorporation conditions (50 mM Glycine pH 9.8 at 60° C.) was measured by staging 3′-ABF deoxythymine to evaluate the stability of 3′-ABF as a blocking group. At set time points, 40 μL aliquots were taken and analyzed by UPLC (conditions/column) to determine the percentage of blocked nucleotide remaining and the eventual formation of unblocked nucleotide.

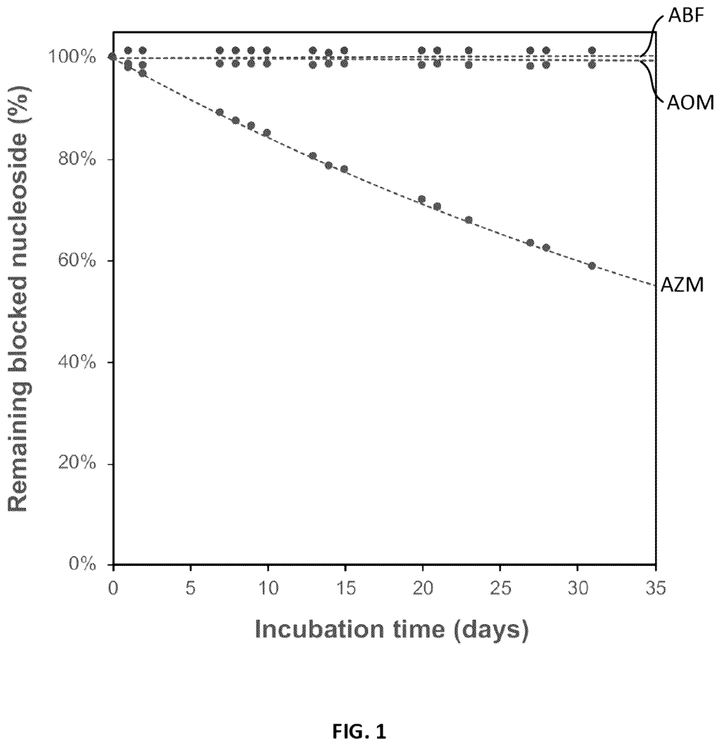

FIG. 1 plots the percentage of remaining blocked nucleoside as a function of time for 3′-ABF-dT, as compared to 3′-AZM-dT and 3′-AOM-dT respectively. About 100% of the 3′-ABF blocked nucleoside was still present after 31 days of staging. This represents similar or better stability compared to AOM and 80-100 fold higher stability of 3′-blocking group compared to AZM in solution. The experimental conditions mimic how the corresponding fully functionalized nucleotides (ffNs) would behave when stored in a liquid incorporation mix in the cartridge of a sequencing device. The stability demonstrated may allow for ambient shipping, storage, and longer shelf life of blocked nucleotides. Additionally, the demonstrated stability may lead to a low pre-phasing rate, reduced signal decay, and lower error rates, which all could contribute to better accuracy of base calls, higher data quality, and higher Q-scores from sequencing data.

### Example 4. Enzymatic Cleavage of 3′-ABF Blocking Group

Candidate arabinofuranosidases (ABFases) were tested for hydrolysis of 3′-ABF. 3′-ABF deoxythymidine was preincubated in 100 μM sodium citrate pH 5.5 or sodium phosphate pH 6.5 at designated temperatures for 5 minutes. Enzyme was added to the reaction so that the final reaction contained 200 μM substrate and 2 μM enzyme. At indicated time points, 50 μL aliquots were removed and quenched with 50 μL 0.1N NaOH prior to analysis by HPLC (YMC Triart C18, 250×4.5 mm, S-5 μm, 12 nm, 0.1M TEAB/100% Acetonitrile) to determine the percentage of enzymatically unblocked nucleotide (or % deprotection).

Various arabinofuranosidases (ABFases) included CAZy Glycoside Hydrolases family 51 (GH51) from Thermotoga maritima, Cellvibrio japonicus, Clostridium thermocellum, Bacillus subtilis, and Podosporina anserina. The molar ratio of enzyme to 3′-ABF deoxythymidine was 1:100 in all reaction mixes. The reaction using T. maritima ABFase was carried out at 80° C. with a sodium phosphate buffer to ensure a pH of 6.5. The reaction using C. japonicus ABFase was carried out at 37° C. with a sodium citrate buffer to ensure a pH of 5.5. The reaction using C. thermocellum ABFase was carried out at 65° C. with a sodium phosphate buffer to ensure a pH of 6.5. The reaction using B. subtilis ABFase was carried out at 60° C. with a sodium phosphate buffer to ensure a pH of 6.5. The reaction using P. anserina ABFase was carried out at 60° C. with a sodium citrate buffer to ensure a pH of 5.5.

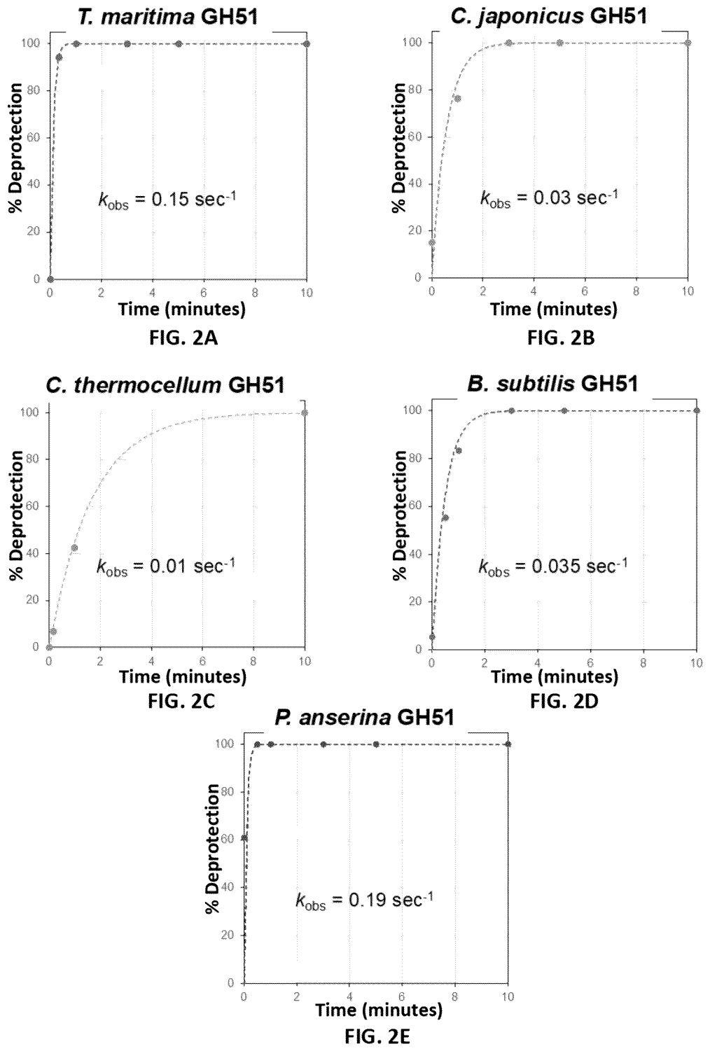

FIGS. 2A-2E are kinetic plots showing the percentage of deoxythymidine generated over the course of the 3′-ABF enzymatic cleavage reaction with the candidate GH51 ABFases. The observed reaction constant k_{obs} for T. maritima GH51 was 0.15 s⁻¹. The observed reaction constant k_{obs} for C. japonicus GH51 was 0.03 s⁻¹. The observed reaction constant k_{obs} for C. thermocellum GH51 was 0.01 s⁻¹. The observed reaction constant k_{obs} for B. subtilis GH51 was 0.035 s⁻¹. The observed reaction constant k_{obs} for P. anserina GH51 was 0.19 s⁻¹.

### Example 5. Solution Kinetic Testing of Enzymatic Cleavage of 3′-ABF Blocked DNA

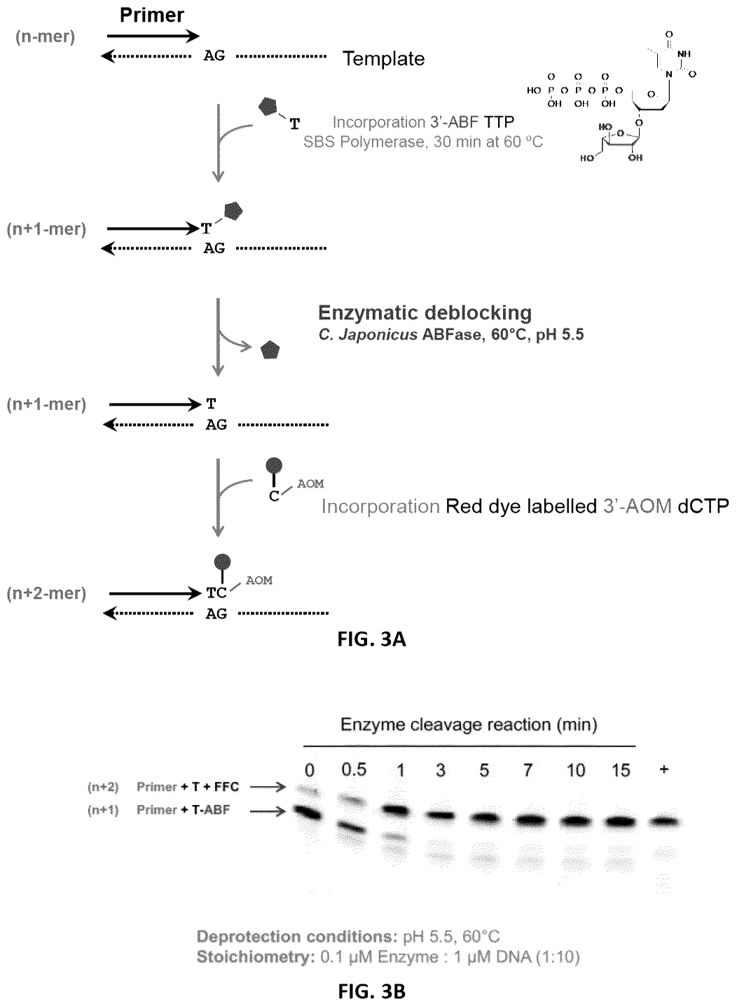

For solution kinetic testing of 3′-ABF cleavage from DNA, a primer:template duplex was prepared as the substrate. A synthetic DNA template was designed to include a sequence complementary to the incorporation of a T followed by a C, ensuring stepwise extension. A DNA primer, 5′-labeled with FAM (fluorescein amidite), was annealed to the template. The primer was first extended by 3′-ABF TTP using a mutant of 9° N DNA polymerase Pol A (SBS polymerase) (e.g., 1 M duplex, 10 μM 3′-ABF TTP, incubation at 60° C. for 30 minutes at pH 9.6). The 3′-ABF extended product was purified to serve as the substrate for kinetic testing. Enzymatic cleavage of the 3′-ABF was performed in 50 mM sodium citrate pH 5.5 at 60° C. using C. japonicus ABFase (0.1 μM) with 3′-ABF blocked primer:template duplex (1 μM). Time-course experiments were performed by quenching aliquots at defined time intervals. Quenched samples were purified and used for further extension with a red dye-labeled 3′-AOM dCTP by SBS Polymerase. The efficiency of cleavage was assessed by analyzing the extension to the next nucleotide using denaturing polyacrylamide gel electrophoresis (PAGE) to resolve the fully extended product. The fluorescent signal from the 5′-FAM-labeled primer was used for visualization and quantification of the substrate and product bands. Product formation over time was quantified using fluorescence detection or densitometric analysis of gel bands. FIG. 3A illustrates the workflow of the described experiment which mimics a SBS cycle in solution.

FIG. 3B is the electrophoresis image of cleaved 5′-fluorescently labeled primers which were subjected to the experimental conditions illustrated in FIG. 3A. The gel image shows that enzymatic cleavage of 3′-ABF from DNA was feasible within 3 minutes at 60° C. using C. Japonicus ABFase. The enzyme cleavage reaction was carried out at pH 5.5, with a 1:10 Enzyme:DNA ratio (0.1 μM ABFase:1 μM DNA duplex).

Other tested ABFases (i.e., thermostable enzymes from Thermotoga maritima and Clostridium thermocellum) were also found to cleave 3′-ABF from DNA under the same experimental conditions.

### Example 6. Enzymatic Cleavage of 3′-ABF from DNA Clusters on a Sequencing Flowcell

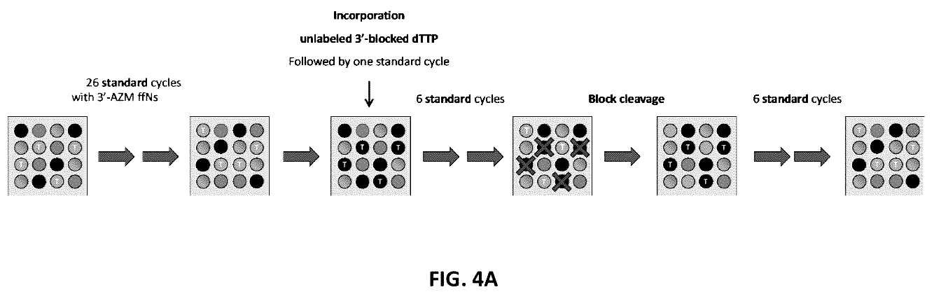

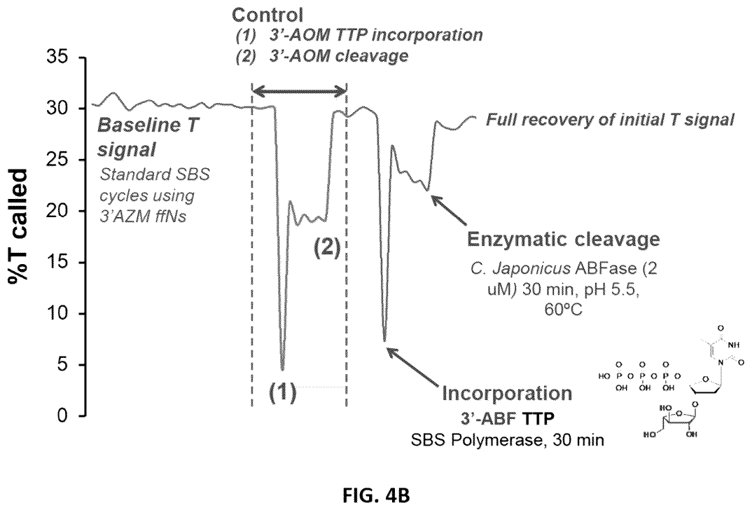

A further example examined whether cleavage of 3′-ABF is feasible on DNA clusters on the surface of a sequencing flow cell. To this end, a sequencing assay monitoring the loss and recovery of % T call signal as read-out was carried out using an Illumina NextSeq 2000 instrument with a P2 flow cell. FIG. 4A illustrates the sequencing assay workflow using loss and recovery of % T call signal as readout, including: (1) The % T call signal baseline based on a standard Illumina Green ffT (e.g., ffT-3′-AZM-LN3-AF550POPOso) is first established with a series of SBS cycles using standard SBS chemistry; (2) incorporation by Pol A (SBS polymerase) of unlabeled dTTP with a different 3′-block (i.e., 3′-ABF dTTP or 3′-AOM dTTP used as control) results in the loss of this signal; (3) the % T signal is only partially recovered to around 75% relative to the initial baseline in subsequent standard cycles as around 25% DNA clusters are blocked by the alternative 3′-block which is uncleaved under standard SBS chemistry conditions; (4) injection of the cleavage mix specifically removing this block (i.e., ABFase removing the 3′-ABF or Pd catalyst removing 3′-AOM for the control experiment) followed by standard cycles resulting in the full recovery of the signal. This assay can thus be used to evaluate the efficiency of both incorporation of a nucleotide with the assayed 3′-block and subsequent removal of that block.

FIG. 4B is a plot of % T call signal of the SBS workflow described in FIG. 4A, plotting percentage of standard green ffT called via fluorescence imaging of the flowcell surface over the consecutive SBS cycles performed on Illumina's NextSeq2000 instrument. This experiment shows that the % T fluorescence signal established from initial standard SBS cycles fully drops upon incorporation of the unlabeled 3′-ABF TTP then partially recovers to around 75% of the initial baseline when standard SBS cycles resumes. Enzymatic cleavage using C. japonicum ABFase (30 minutes, 60° C., pH 5.5) then removes the 3′-ABF on the clusters previously blocked (corresponding to 25% of the total clusters) and enables full recovery of the initial % T fluorescence signal. All together, these results demonstrate successful enzymatic cleavage of the 3′ ABF block from DNA clusters using a sequencing instrument.

### Example 7. Surface Kinetic of 3′-ABF Cleavage from DNA Catalyzed by an Arabinofuranosidase (ABFase)

An assay primer (dubbed HP10 with sequence 5′-ACACTCTTTCCCTACACGACGCTCTTCCGATCT-3′ (SEQ ID NO:1)) was prepared to serve as substrate for 3′-ABF enzymatic cleavage. This primer was generated by extending an initiator (with sequence 5′-ACACTCTTTCCCTACACGACGCTCTTCCGATC-3′ (SEQ ID NO:2)) using 3′-ABF TTP, SBS Polymerase Pol A and a template allowing for the incorporation of a T (under similar conditions as described in Example 5). The resulting 3′-ABF HP10 primer was then purified and prepared for the next step.

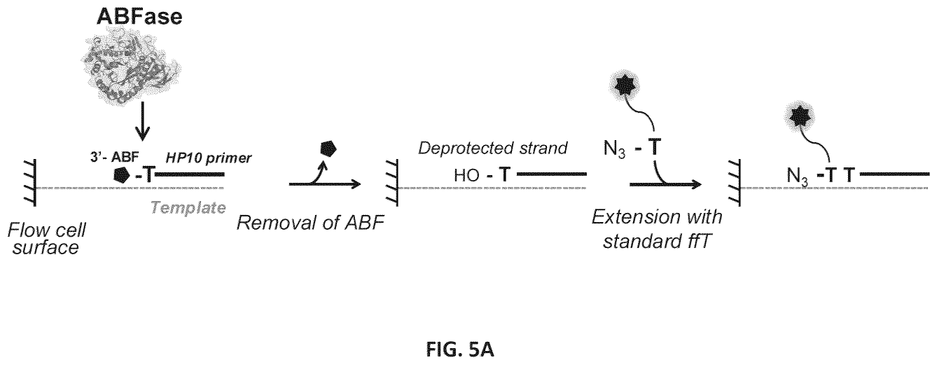

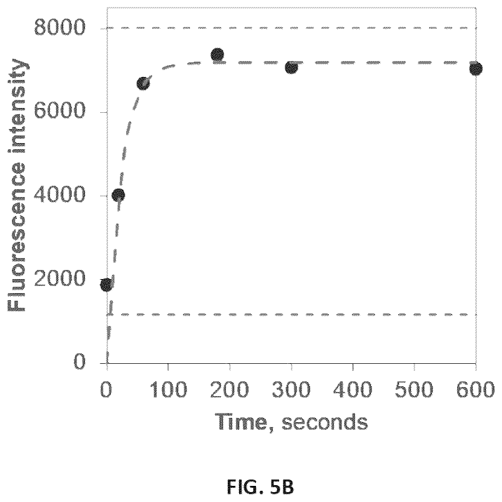

FIG. 5A illustrates the experimental workflow used to perform surface kinetics of 3′-ABF enzymatic cleavage from DNA using the prepared 3′-ABF HP10 primer. The experiment was carried out using an Illumina HiSeq® X 8-lane flowcell in an Illumina cBot equipped with a fluorescence imaging camera. The surface-bound primers on the flowcell were initially extended using a high-fidelity DNA polymerase and a complementary oligonucleotide to generate the template complementary to HP10 and allowing for further extension by a standard Illumina green ffT. Two lanes of the flowcell were used for the two following controls: (1) hybridization with 3′-OH HP10 followed by extension with a standard Green ffT for maximum fluorescence measurement; (2) hybridization with 3′-OH HP10 with no further extension to measure background. For surface kinetics of the 3′ABF cleavage, primer 3′-ABF HP10 was injected into the six remaining lanes for hybridization onto the surface-attached DNA template. Enzymatic cleavage of 3′-ABF was performed by injecting ABFase from C. japonicus (2 μM) in its reaction buffer (50 mM sodium citrate pH 5.5) into five of these lanes and incubating at 50° C. At defined intervals (e.g., 20 seconds, 1, 3, 5, and 10 minutes of contact time), the reaction was stopped by injecting wash buffer (e.g., 50 mM Tris-HCl pH 9.2, 50 mM NaCl, 0.05% Tween-20) or continued with a sequential injection of ABFase to achieve longer cleavage times. The remaining lane was used as a negative control (injection of buffer only) and as zero time point in the kinetics. Further extension with a standard Green ffT onto the unblocked strands followed by fluorescence imaging was used as a read-out for cleavage efficiency, as only primers with the 3′-ABF removed by the ABFase could be extended.

FIG. 5B shows the kinetic plot of 3′-ABF cleavage by ABFase over reaction contact time. Fluorescence intensity refers to the amount of red dye labelled ffC incorporated and detected upon enzymatic cleavage.

### Example 8. Incorporation of 5′-triphosphate 3′-glycopyranoside Thymidine by SBS DNA Polymerases

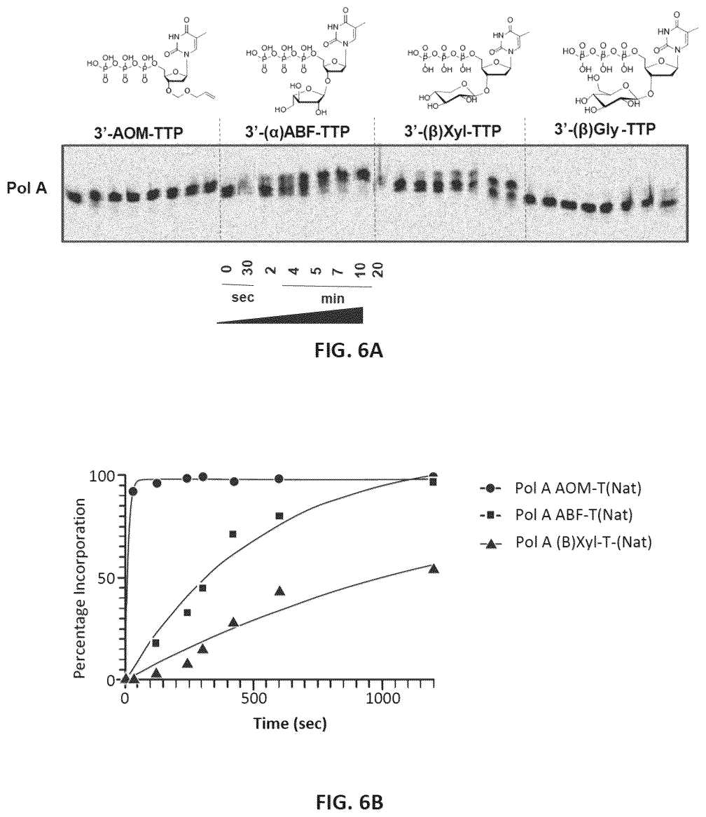

The incorporation of the 5′-triphosphate 3′-[(β)-D-glucopyranosyl]thymidine and 5′-triphosphate 3′-[(β)-D-xylopyranosyl]thymidine into DNA by a mutant DNA polymerase Pol A was tested using a primer extension assay. Briefly, a ³³P-labelled DNA primer:template (20 nM) was incubated with the DNA polymerase (335 mg/mL) and the nucleotide triphosphates (1 μM) in buffer (50 mM Glycine pH 9.6, 50 mM NaCl, 9 mM MgSO₄, 1 mM EDTA) at 60° C. The reaction was quenched at set time points by the addition of EDTA and analyzed by PAGE electrophoresis as shown in FIG. 6A. The rate was determined by calculating the percentage of incorporation compared to the initial primer template band and the incorporation results are shown in FIG. 6B. The results show that 5′-triphosphate 3′-1(β)-D-xylopyranosyl]thymidine is a substrate for Pol A, but the rate of incorporation is slower compared to 5′-triphosphate 3′-[(α)-L-arabinofuranosyl]thymidine and 5′-triphosphate 3′-allyloxymethyl thymidine. 5′-triphosphate 3′-[(β)-D-glucopyranosyl]thymidine is a poor substrate for Pol A, with no detectable incorporation observed under these conditions.

Cleavage 3′-glucopyranoside Thymidine by Glycosyl Hydrolases

The cleavage of the 3′-glycoside moiety from 3′-(β)-glucopyranoside thymidine, 3′-(β)-xylopyranoside thymidine and 3′-(α)-mannopyranoside thymidine using glycosyl hydrolases was tested by incubating the nucleoside substrate (200 μM) with the enzyme (2 μM) in buffer. At set timepoints, 50 μL aliquots of the reaction solution were taken and quenched with 50 μL of either 0.1 M EDTA or 0.1 μM NaOH. The aliquots were then analysed by reverse phase HPLC (YMC-Triart C18 Column, 12 nm, 5 μm, 250×4.6 mm column) using a gradient from 0% to 8% acetonitrile in 0.1 M TEAB over 25 minutes, monitoring the disappearance of the 3′-glycopyranoside thymidine substrate peak and the appearance of the thymidine product peak. The conditions used for each enzyme and substrate are summarized in Table 2 below.

TABLE 2
Glycosyl hydrolases cleavage conditions

| Glycosyl hydrolase | Substrate | Buffer | Temperature |
|---|---|---|---|
| β-Glucosidase 1A, | 3′-(β)-glucopyranoside | 100 mM HEPES | 50° C. |
| Thermobifida fusca | thymidine | pH 7.0 | |
| β-Glucosidase 1A, Thermus | 3′-(β)-glucopyranoside | 100 mM sodium | 50° C. |
| thermophilus | thymidine | citrate pH 6.5 | |
| β-Glucosidase 3B, | 3′-(β)-glucopyranoside | 100 mM sodium | 37° C. |
| Cellvibrio japonicus | thymidine | phosphate pH | |
| | | 7.5 | |
| β-Glucosidase 3B, | 3′-(β)-glucopyranoside | 100 mM sodium | 37° C. |
| Cellvibrio japonicus | thymidine | citrate pH 6.5 | |
| Xylosidase 3A, Sulfolobus | 3′-(β)-xylopyranoside | 100 mM MES | 50° C. |
| solfataricus | thymidine | pH 6.0 | |
| Xylosidase 39A, | 3′-(β)-xylopyranoside | 100 mM MES | 50° C. |
| Clostridium stercorarium | thymidine | pH 6.0, 5 mM | |
| | | CaCl₂ | |
| Xylosidase 43A, | 3′-(β)-xylopyranoside | 100 mM sodium | 50° C. |
| Thermobifida fusca | thymidine | citrate pH 5.5 | |
| α-Mannosidase 92S, | 3′-(α)-mannopyranoside | 100 mM HEPES | 37° C. |
| Bacteroides | thymidine | pH 7.0, 2 mM | |
| thetaiotaomicron | | CaCl₂ | |
| α-Mannosidase 92B, | 3′-(α)-mannopyranoside | 100 mM HEPES | 37° C. |
| Bacteroides | thymidine | pH 7.0, 2 mM | |
| thetaiotaomicron | | CaCl₂ | |

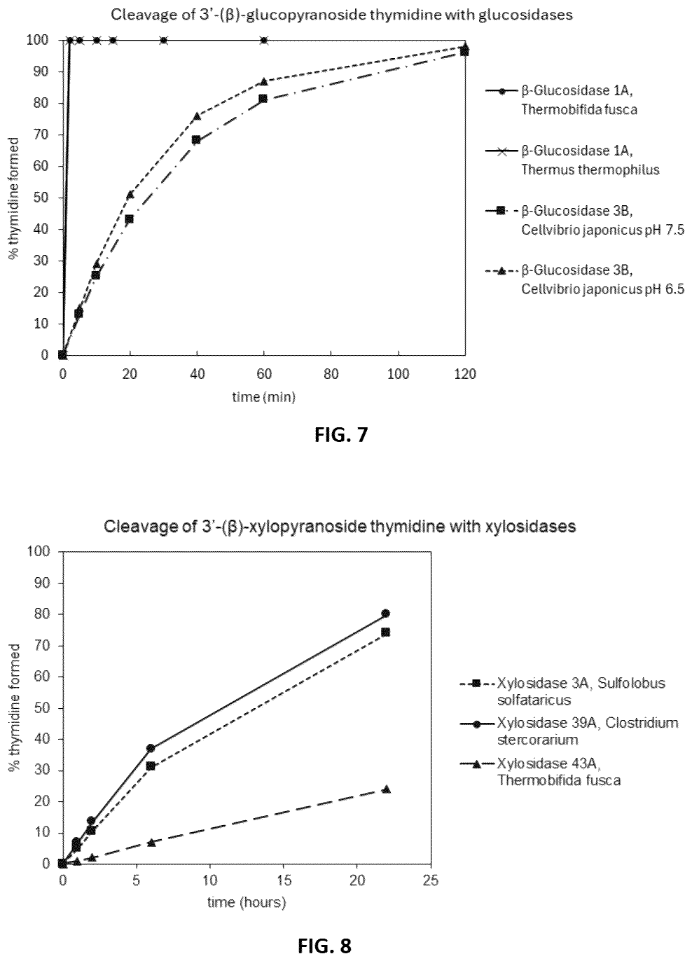

The cleavage kinetics are shown in FIGS. 7 and 8. The cleavage of the 3′-glucopyranoside group by the enzymes β-Glucosidase 1A, Thermobifida fusca and β-Glucosidase 1A, Thermus thermophilus was complete in less than 2 minutes, under the conditions tested. 3-Glucosidase 3B, Cellvibrio japonicus achieved complete cleavage in approximately 2 hours at both pH 7.5 and 6.5 (see FIG. 7). Cleavage of the 3′-xylopyranoside group was observed with the three xylosidase enzyme tested but none of the enzymes achieved full conversion of the substrate within 22 hours, under the conditions tested (see FIG. 8). Cleavage of the 3′-mannopyranoside group was not observed with two mannosidases from Bacteroides thetaiotaomicron, under the conditions tested.

1. A nucleotide comprising a ribose or 2′ deoxyribose having an enzymatically removable 3′ blocking group in the form of a 3′-O-glycoside group, a nucleobase and a triphosphate moiety, wherein the 3′ blocking group forms an —O-glycosidic bond with the 3′ carbon atom of the nucleotide.

2. The nucleotide of claim 1, wherein 3′ blocking group has the structure: 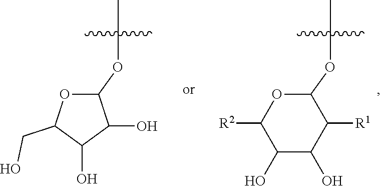
wherein the squiggle line indicates the point of attachment to the 3′-carbon atom, wherein:
R¹ is OH, or —NHR³;
R² is H, —CH₂OH, or —C(═O)OR⁴;
R³ is H or an amino protecting group; and
R⁴ is H or C₁-C₆ alkyl.

3. The nucleotide of claim 1, wherein the removable 3′ blocking group has a structure:

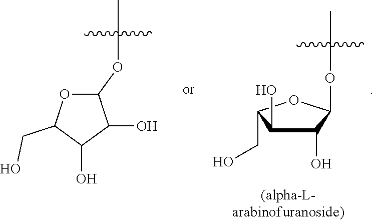

4. The nucleotide of claim 1, wherein the removable 3′ blocking group has a structure:

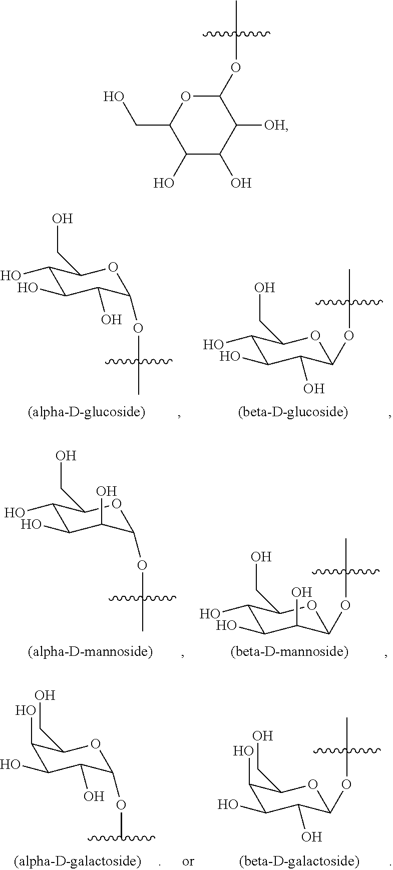

5. The nucleotide of claim 1, wherein the removable 3′ blocking group has a structure:

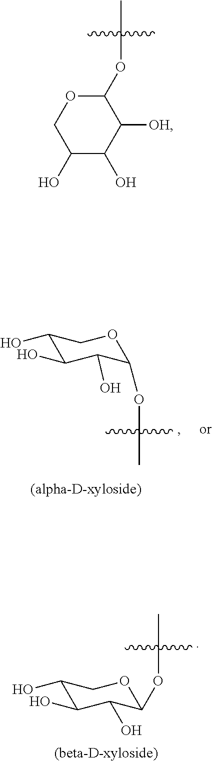 [SMILES: *OC1OCC(O)C(O)C1O ; *OC1OCC(O)C(O)C1O ; *OC1OCC(O)C(O)C1O]

6. The nucleotide of claim 1, wherein the removable 3′ blocking group has a structure:

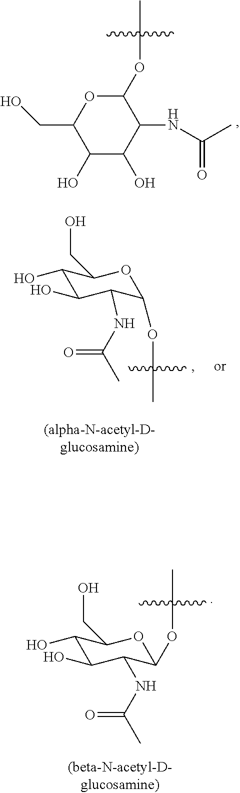

7. The nucleotide of claim 1, wherein the removable 3′ blocking group has a structure:

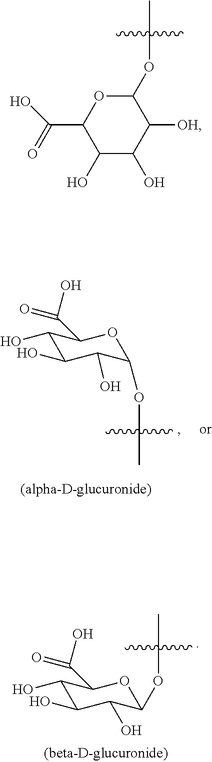 [SMILES: *OC1OC(C(=O)O)C(O)C(O)C1O ; *OC1OC(C(=O)O)C(O)C(O)C1O ; *OC1OC(C(=O)O)C(O)C(O)C1O]

8. The nucleotide of claim 1, further comprising a detectable label.

9. The nucleotide of claim 8, wherein the detectable label is a fluorescent dye.

10. The nucleotide of claim 8, wherein the detectable label is connected to the nucleobase via a cleavable linker.

11. The nucleotide of claim 10, wherein the cleavable linker is enzymatically cleavable.

12. The nucleotide of claim 10, wherein the cleavable linker is an enzyme-triggered self-immolative linker.

13. The nucleotide of claim 10, wherein the detectable label and the 3′ blocking group are removable by a single enzymatic reaction.

14. An oligonucleotide or polynucleotide comprising a nucleotide of claim 1 incorporated therein.

15. The oligonucleotide or polynucleotide of claim 14, wherein the oligonucleotide or polynucleotide is at least partially complementary and hybridized to a target polynucleotide immobilized on a surface of a solid support.

16. (canceled)

17. A kit comprising one or more nucleotide according to claim 1.

18. The kit of claim 17, further comprising a first enzyme, wherein the first enzyme is a polymerase, a terminal deoxynucleotidyl transferase, or a reverse transcriptase.

19. (canceled)

20. The kit of claim 18, further comprising a second enzyme for removing the 3′ blocking group of the nucleotide, wherein the second enzyme is in a separate compartment from the first enzyme.

21. The kit of claim 20, wherein the second enzyme is a glycoside hydrolase or glycosidase that is capable of catalyzing the hydrolysis of the —O-glycosidic bond of the nucleotide.

22. The kit of claim 21, wherein the second enzyme is an arabinofuranosidase, a glucosidase, a mannosidase, a xylosidase, a galactosidase, an N-acetyl-glucosaminidase, or a glucuronidase.

23. A method of preparing a growing polynucleotide complementary to a target single-stranded polynucleotide in a sequencing reaction, comprising incorporating a nucleotide of claim 1 into a growing complementary polynucleotide, wherein the incorporation of the nucleotide prevents the introduction of any subsequent nucleotide into the growing complementary polynucleotide.

24. (canceled)

25. A method of determining the sequences of a plurality of target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an aqueous solution comprising DNA polymerase and one or more of four different types of nucleotides A, G, C, and T or U under conditions suitable for DNA polymerase-mediated primer extension, wherein each type of nucleotides has a 3′ blocking group and at least one type of nucleotide is a nucleotide of claim 1, and wherein each of the one or more of four different type of nucleotides comprises a 2′ deoxyribose;
(c) incorporating one type of nucleotides into the sequencing primers to produce extended copy polynucleotides; and
(d) imaging the solid support and performing one or more fluorescent measurements of the extended copy polynucleotides; and
(e) removing the 3′ blocking group from the nucleotides incorporated into the extended copy polynucleotides.

26.-34. (canceled)


SEQUENCE LISTING (2 sequences)

SEQ ID NO: 1 - DNA, 33 bp, synthetic construct
  source 1..33: mol_type = other DNA
acactctttccctacacgacgctcttccgatct

SEQ ID NO: 2 - DNA, 32 bp, synthetic construct
  source 1..32: mol_type = other DNA
acactctttccctacacgacgctcttccgatc